(19) United States
(12) Patent Application Publication
(10) Pub. No.: US 2023/0014360 A1
(43) Pub. Date: Jan. 19, 2023

(54) Electronic Device

(71) Applicant: Semiconductor Energy Laboratory Co., Ltd., Kanagawa-ken (JP)

(72) Inventors: Hajime Kimura, Kanagawa-ken (JP); Rihito WADA, Kanagawa-ken (JP)

(73) Assignee: Semiconductor Energy Laboratory Co., Ltd., Kanagawa-ken (JP)

(21) Appl. No.: 17/783,161

(22) PCT Filed: Nov. 30, 2020
(86) PCT No.: PCT/IB2020/061254

§ 371 Date: Jun. 7, 2022

(30) Foreign Application Priority Data
Dec. 13, 2019 (JP) 2019-225818

(51) Int. Cl. A61B 5/16; G06N 3/04; G06F 1/16; A61B 5/00

(52) U.S. Cl. CPC A61B 5/165; G06N 3/0454; G06F 1/163; A61B 5/6803

(57) ABSTRACT

To provide an electronic device capable of recognizing a user's emotion with a high accuracy. The electronic device includes a detection device, an arithmetic device, and a housing. The housing includes a space at a position overlapping with a user's nose when the user wears the electronic device. The detection device is located between the housing and the user's nose. The detection device has a function of obtaining user's data on an emotion of the user and outputting the user's data to the arithmetic device. The arithmetic device has a function of generating display data based on the user's data and outputting the display data.

### TECHNICAL FIELD

One embodiment of the present invention relates to electronic devices.

Note that one embodiment of the present invention is not limited to the above technical field. Examples of the technical field of one embodiment of the present invention disclosed in this specification and the like include a semiconductor device, a display device, a light-emitting apparatus, a power storage device, a memory device, an electronic device, a lighting device, an input device, an input/output device, a driving method thereof, and a manufacturing method thereof. A semiconductor device refers to a device that can function by utilizing semiconductor characteristics in general.

### BACKGROUND ART

As display devices for augmented reality (AR) or virtual reality (VR), wearable display devices and stationary display devices are becoming widespread. Examples of wearable display devices include a head mounted display (HMD) and an eyeglass-type display device. Examples of stationary display devices include a head-up display (HUD).

A technique which obtains information of a body motion or a facial expression of a user with use of a sensor, a camera, or the like provided on a head-mounted display and displays the information has been considered. Patent Document 1 and Patent Document 2 each disclose a structure in which a camera is provided in a head-mounted display to recognize a facial expression of a user.

### REFERENCE

### Patent Document

[Patent Document 1] PCT International Publication No. 2017/122299
[Patent Document 2] Japanese Translation of PCT International Application No. 2018-538593

### SUMMARY OF THE INVENTION

### Problems to be Solved by the Invention

In the case where an electronic device is provided with a detection device to obtain information on a user's emotion, and the detection device is apart from the user, the detection accuracy is decreased and the emotion of the user is unlikely to be detected with a high accuracy. In addition, when the detection device is protruded from the housing of the electronic device, the reliability of the electronic device might be decreased.

An object of one embodiment of the present invention is to provide an electronic device capable of recognizing a user's emotion with a high accuracy. Another object of one embodiment of the present invention is to provide an electronic device capable of estimating the kind or the degree of a user's emotion with a high accuracy. Another object of one embodiment of the present invention is to provide a highly reliable electronic device. Another object of one embodiment of the present invention is to provide a novel electronic device.

Note that the description of these objects does not preclude the existence of other objects. One embodiment of the present invention does not need to achieve all the objects listed above. Note that other objects can be derived from the description of the specification, the drawings, the claims, and the like.

### Means for Solving the Problems

One embodiment of the present invention is an electronic device including a detection device, an arithmetic device, and a housing. The housing includes a space at a position overlapping with a user's nose when the user wears the electronic device. The detection device is located between the housing and the user's nose. The detection device has a function of obtaining user's data on an emotion of the user and outputting the user's data to the arithmetic device. The arithmetic device has a function of generating display data based on the user's data and outputting the display data.

One embodiment of the present invention is an electronic device including a detection device, an arithmetic device, and a housing. The housing includes a space at a position overlapping with a user's nose when the user wears the electronic device. The detection device is located in the inside of the housing to overlap with the user's nose. The detection device has a function of obtaining user's data on an emotion of the user and outputting the user's data to the arithmetic device. The arithmetic device has a function of generating display data based on the user's data and outputting the display data.

One embodiment of the present invention is an electronic device including a detection device, an arithmetic device, a display device, and a housing. The housing includes a space at a position overlapping with a user's nose when the user wears the electronic device. The detection device is located between the housing and the user's nose. The detection device has a function of obtaining user's data on an emotion of the user and outputting the user's data to the arithmetic device. The arithmetic device has a function of generating display data based on the user's data and outputting the display data to the display device.

One embodiment of the present invention is an electronic device including a detection device, an arithmetic device, a display device, and a housing. The housing includes a space at a position overlapping with a user's nose when the user wears the electronic device. The detection device is located in the inside of the housing to overlap with the user's nose. The detection device has a function of obtaining user's data on an emotion of the user and outputting the user's data to the arithmetic device. The arithmetic device has a function of generating display data based on the user's data and outputting the display data to the display device.

In the above-described electronic devices, the detection device preferably includes one or more of a temperature sensor, a humidity sensor, a microphone, and an imaging device.

In the above-described electronic devices, the user's data is preferably one or more of a temperature, a humidity, a sound, and an image.

The above-described electronic devices each further include an adjustment mechanism. The adjustment mechanism has a function of adjusting an angle of the detection device with respect to the housing.

In the above-described electronic devices, the detection device preferably includes an imaging device. The detection device has a function of outputting, to the arithmetic device, a captured image of the user as the user's data. The arithmetic device has a function of estimating an emotion of the user from the user's data and generating the display data based on the estimated emotion.

In the above-described electronic devices, the user's data is preferably an image of a portion including the user's nose.

In the above-described electronic devices, the user's data is preferably an image of a portion including the user's mouth.

In the above-described electronic devices, a neural network is preferably used for the estimation.

### Effect of the Invention

One embodiment of the present invention can provide an electronic device which can recognize a user's emotions with a high accuracy. Another embodiment of the present invention can provide an electronic device which can estimate the kind or the degree of a user's emotion with a high accuracy. Another embodiment of the present invention can provide an electronic device with a high reliability. Another embodiment of the present invention can provide a novel electronic device.

Note that the description of these effects does not preclude the existence of other effects. One embodiment of the present invention does not need to have all the effects listed above. Other effects can be derived from the description of the specification, the drawings, the claims, and the like.

### BRIEF DESCRIPTION OF THE DRAWINGS

FIG. 1A and FIG. 1B are external views illustrating a structure example of an electronic device.

FIG. 2A and FIG. 2B are external views illustrating a structure example of the electronic device.

FIG. 3A and FIG. 3B are block diagrams each illustrating a structure example of the electronic device.

FIG. 4A to FIG. 4C are external views illustrating a structure example of a housing.

FIG. 5A and FIG. 5B are external views illustrating a structure example of the electronic device.

FIG. 6A and FIG. 6B are external views illustrating a structure example of the electronic device.

FIG. 7A and FIG. 7B are diagrams illustrating the housing and a detection device.

FIG. 8A and FIG. 8B are external views illustrating a structure example of the electronic device.

FIG. 9A and FIG. 9B are external views each illustrating a structure example of the electronic device.

FIG. 10A is an external view showing a structural example of the electronic device. FIG. 10B is a block diagram illustrating a structure example of the electronic device.

FIG. 11A and FIG. 11B are external views illustrating a structure example of an electronic device.

FIG. 12 is a block diagram illustrating a structure example of the electronic device.

FIG. 13 is an external view illustrating a structure example of the electronic device.

FIG. 14A and FIG. 14B are external views illustrating a structure example of the electronic device.

FIG. 15 is an external view illustrating a structure example of an electronic device.

FIG. 16A and FIG. 16B are external views illustrating a structure example of the electronic device.

FIG. 17 is a block diagram illustrating a structure example of an arithmetic device.

FIG. 18A and FIG. 18B illustrate a structure example of a neural network. FIG. 18C is a diagram describing estimation of emotions.

FIG. 19A1 to FIG. 19A4 and FIG. 19B1 to FIG. 19B4 each illustrate an example of an image of a portion including a mouth.

FIG. 20A and FIG. 20B illustrate examples of a user's field of view.

FIG. 21A and FIG. 21B illustrate examples of a user's field of view.

FIG. 22A to FIG. 22C each illustrate a structure example of a housing.

FIG. 23A and FIG. 23B each illustrate a structure example of a housing.

FIG. 24A and FIG. 24B are external views each illustrating a structure example of a housing.

FIG. 25 is a block diagram illustrating a structure example of a display device.

FIG. 26 is a block diagram illustrating a configuration example of the display device.

FIG. 27A to FIG. 27G each illustrate an example of a pixel configuration.

FIG. 28A and FIG. 28B are circuit diagrams each illustrating an example of a pixel configuration.

FIG. 29A is a circuit diagram illustrating an example of a pixel configuration. FIG. 29B is a timing chart illustrating an example of a pixel operation.

FIG. 30A to FIG. 30E are circuit diagrams each illustrating an example of a pixel configuration.

FIG. 31 is a block diagram illustrating a structure example of the display device.

FIG. 32 is a diagram illustrating an operation example of the display device.

FIG. 33 is a cross-sectional view illustrating a structure example of the display device.

FIG. 34 is a cross-sectional view illustrating a structure example of the display device.

FIG. 35 is a cross-sectional view illustrating a structure example of the display device.

FIG. 36 is a cross-sectional view illustrating a structure example of the display device.

FIG. 37A to FIG. 37E illustrate structure examples of light-emitting devices.

FIG. 38A and FIG. 38B are cross-sectional views illustrating structure examples of an imaging device.

FIG. 39A is a top view illustrating a structure example of a transistor. FIG. 39B and FIG. 39C are cross-sectional views illustrating the structural example of the transistor.

FIG. 40A is a top view illustrating a structure example of a transistor. FIG. 40B and FIG. 40C are cross-sectional views illustrating the structural example of the transistor.

FIG. 41A is a top view illustrating a structure example of a transistor. FIG. 41B and FIG. 41C are cross-sectional views illustrating the structural example of the transistor.

### MODE FOR CARRYING OUT THE INVENTION

Embodiments will be described below with reference to the drawings. Note that the embodiments can be implemented with many different modes, and it will be readily understood by those skilled in the art that modes and details thereof can be changed in various ways without departing from the spirit and scope thereof. Therefore, the present invention should not be construed as being limited to the description of embodiments below.

Note that in structures of the invention described below, the same portions or portions having similar functions are denoted by the same reference numerals in different drawings, and the description thereof is not repeated. The same hatching pattern is used for portions having similar functions, and the portions are not denoted by specific reference numerals in some cases.

Note that in each drawing described in this specification, the size, the layer thickness, or the region of each component is exaggerated for clarity in some case. Therefore, the size, the layer thickness, or the region is not limited to the illustrated scale.

Note that in this specification and the like, ordinal numbers such as “first” and “second” are used in order to avoid confusion among components and do not limit the number of components.

In this specification, terms for describing arrangement, such as “over”, “above”, “under”, “below”, “left”, and “right”, are used for convenience in describing a positional relation between components with reference to drawings. The positional relation between components is changed as appropriate in accordance with the direction from which each component is described. Thus, the positional relation is not limited to the positional relation described with a term used in this specification and can be described with another term as appropriate depending on the situation.

A transistor is a kind of semiconductor elements and can achieve amplification of current and voltage, switching operation for controlling conduction and non-conduction, and the like. A transistor in this specification includes, in its category, an IGFET (Insulated Gate Field Effect Transistor) and a thin film transistor (TFT).

In this specification and the like, a function of a source and a drain of a transistor are sometimes replaced with each other depending on the polarity of the transistor or when the direction of current flow is changed in circuit operation, for example. Therefore, the terms source and drain can be used interchangeably.

In this specification and the like, the expression “electrically connected” includes the case where components are directly connected to each other and the case where components are connected through an “object having any electric function”. There is no particular limitation on an “object having any electric function” as long as electric signals can be transmitted and received between components that are connected through the object. Thus, even when the expression “electrically connected” is used, there is a case where no physical connection is made and a wiring just extends in an actual circuit. In addition, the expression “directly connected” includes the case where different conductors are connected to each other through a contact. Note that a wiring may be formed of conductors that contain one or more of the same elements or may be formed of conductors that contain different elements.

Unless otherwise specified, off-state current in this specification and the like refers to a drain current of a transistor in an off state (also referred to as a non-conducting state or a cutoff state). Unless otherwise specified, the off state of an n-channel transistor means that the voltage between a gate and a source (V_{gs}) is lower than the threshold voltage (Vₜₕ) (the off state of a p-channel transistor means that V_{gs} is higher than Vₜₕ).

In this specification and the like, the term such as “electrode” or “wiring” does not limit the components functionally. For example, an “electrode” is used as part of a wiring in some cases, and vice versa. Furthermore, the term “electrode” or “wiring” can include the case where a plurality of electrodes or wirings are formed in an integrated manner.

In this specification and the like, the resistance value of a “resistor” is sometimes determined depending on the length of a wiring. Alternatively, the resistance value is sometimes determined by connection to a conductor with resistivity different from that of a conductor used for a wiring. Alternatively, the resistance value is sometimes determined by doping a semiconductor with an impurity.

In this specification and the like, a “terminal” in an electric circuit refers to a portion that inputs or outputs current or voltage or receives or transmits a signal. Accordingly, part of a wiring or an electrode functions as a terminal in some cases.

In this specification and the like, a metal oxide means an oxide of metal in a broad sense. Metal oxides are classified into an oxide insulator, an oxide conductor (including a transparent oxide conductor), an oxide semiconductor (also simply referred to as OS), and the like. For example, a metal oxide used in an active layer of a transistor is referred to as an oxide semiconductor in some cases. In other words, an OS FET can be described as a transistor including an oxide or an oxide semiconductor.

### Embodiment 1

In this embodiment, an electronic device of one embodiment of the present invention will be described with reference to drawings.

One embodiment of the present invention is an electronic device including a display device, a detection device, an arithmetic device, and a housing. The detection device has a function of obtaining data on an emotion of a user and outputting the data to the arithmetic device. The arithmetic device has a function of generating display data based on the data and outputting the display data to the display device.

For example, as data on a user's emotion, a temperature or a humidity around the user's nose or mouth or an image thereof can be used. When a user of an electronic device gets excited, the temperature or the humidity around his/her nose or mouth may be increased. The degree of the user's excitement can be estimated by obtaining the temperature or the humidity around the user's nose or mouth. The kind or the degree of the user's emotion can be estimated by obtaining an image of his/her mouth. An estimated emotion of the user is displayed on an electronic device, whereby the user can know his/her state and have a high sense of immersion.

An electronic device of one embodiment of the present invention has a space at a position of a user's nose in its housing and a detection device is located in the space. By providing the detection device close to a user's nose, the user's emotion can be recognized with a higher accuracy. Furthermore, when the detection device is provided in the space and the detection device is not protruded from the housing, interference between the user or another object and the detection device can be inhibited and the reliability of the electronic device can be improved.

### Structure Example 1 of Electronic Device

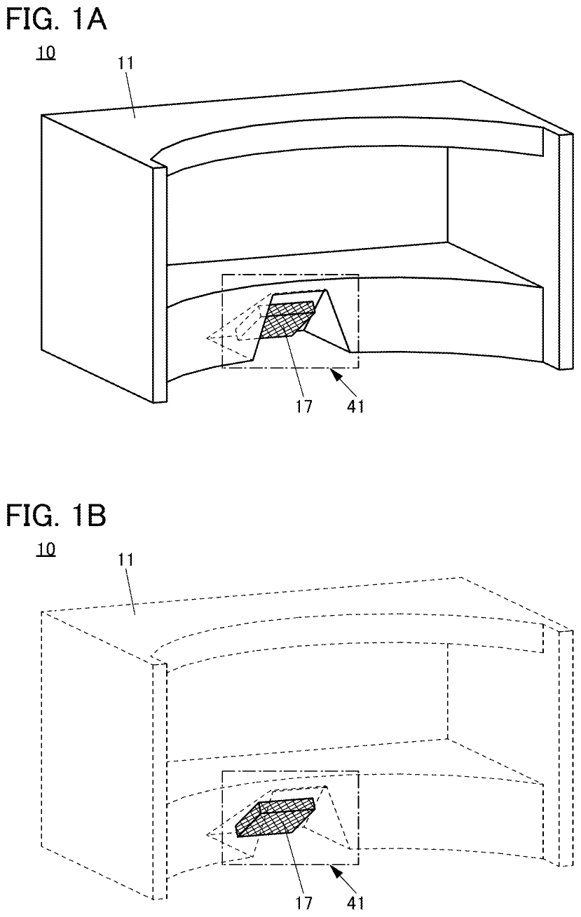

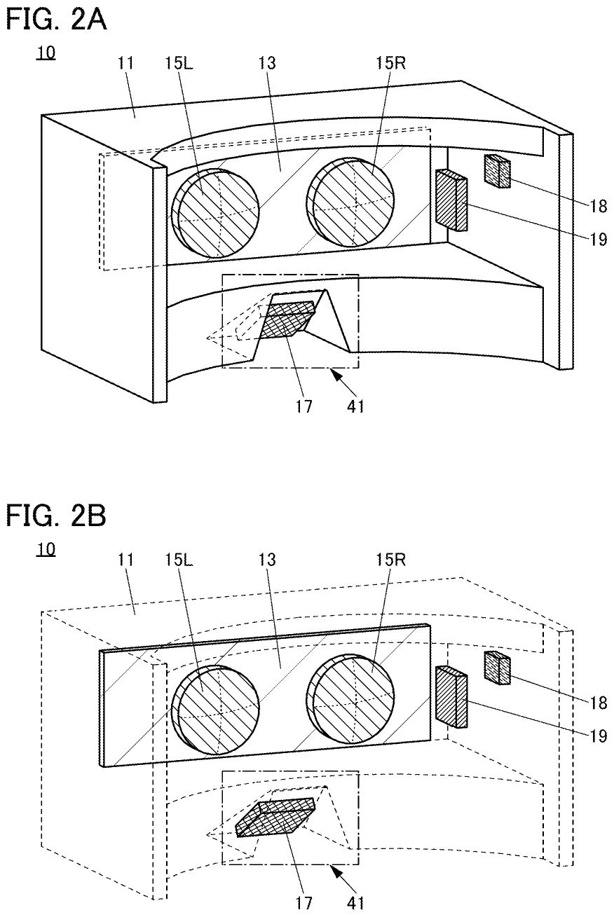

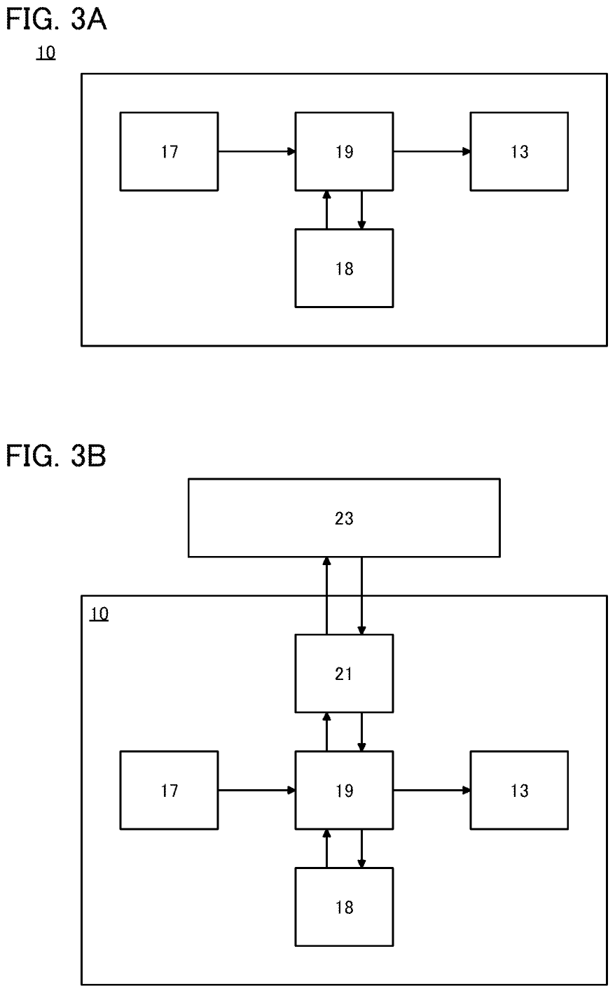

FIG. 1A, FIG. 1B, FIG. 2A, FIG. 2B, and FIG. 3A illustrate a structural example of an electronic device of one embodiment of the present invention. FIG. 1A, FIG. 1B, FIG. 2A, and FIG. 2B are perspective views illustrating the appearance of an electronic device 10. FIG. 3A is a block diagram illustrating a structural of the electronic device 10.

Note that in a block diagram attached to this specification, components are classified according to their functions and shown as independent blocks; however, it is practically difficult to completely separate the components according to their functions, and one component may be related to a plurality of functions or a plurality of components may achieve one function.

The electronic device 10 has a function of displaying an image. The electronic device 10 can be used as a head mounted display (HMD). The electronic device 10 can be favorably used as a display device for displaying an image for augmented reality (AR) or virtual reality (VR). Note that the electronic device 10 can also be called a goggle-type electronic device.

The electronic device 10 includes a housing 11 and a detection device 17 as illustrated in FIG. 1A and FIG. 1B. The housing 11 includes a space 41 in a lower portion, and the detection device 17 is provided in the space 41. The space 41 can also be referred to as a depressed portion of the housing 11. The space 41 is provided at a position overlapping with the user's nose when the user wears the electronic device 10. In FIG. 1B, the housing 11 is illustrated with a dashed line to clearly show the positional relationship between the housing 11 and the detection device 17. The electronic device 10 in FIG. 1A and FIG. 1B can be used as an HMD by being combined with another electronic device having a display portion.

As illustrated in FIG. 2A and FIG. 2B, the electronic device 10 may include a housing 11, a display device 13, a detection device 17, an arithmetic device 19, and a memory device 18. The electronic device 10 may further include an optical component 15L and an optical component 15R. Note that in FIG. 2B, the housing 11 is illustrated with a dashed line in FIG. 2B to clearly show the positional relationship between the housing 11, the display device 13, the detection device 17, the arithmetic device 19, the memory device 18, the optical component 15L, and the optical component 15R.

The display device 13 includes a plurality of pixels and has a function of displaying images. As the display device 13, a display device such as a liquid crystal display device, a light-emitting apparatus (a light-emitting apparatus in which a light-emitting device is provided in each pixel), an electrophoresis display device, a DMD (Digital Micromirror Device), a PDP (Plasma Display Panel), or an FED (Field Emission Display) can be used, for example.

As the light-emitting device, an OLED (Organic Light Emitting Diode), QLED (Quantum-Dot Light-Emitting Diode), or the like preferably used. As a light-emitting substance included in the light-emitting device, a substance emitting fluorescent light (a fluorescent material), a substance emitting phosphorescent light (a phosphorescent material), a substance exhibiting thermally activated delayed fluorescence (a thermally activated delayed fluorescent (TADF) material), an inorganic compound (e.g., a quantum dot material), or the like can be used. An LED (Light Emitting Diode) such as a micro-LED can be used as the light-emitting device.

In the case where the electronic device 10 is used as a head-mounted display, the distance between the user's eyes and the display device 13 is short; thus, the user is likely to perceive pixels and strongly feels granularity, whereby the sense of immersion or realistic emotion of AR and VR might be diminished. Therefore, the display device 13 is preferably a high resolution display device so that pixels are not perceived by the user. The use of the display device 13 with high resolution enables a user of the electronic device 10 to view an image displayed on the display device 13 without feeling the granularity. The resolution of the display device 13 is, for example, preferably 1000 ppi or higher, further preferably 2000 ppi or higher, and still further preferably 5000 ppi or higher. In AR applications, an image of a virtual space is displayed, superimposed on a real space; thus, the display device 13 with high luminance is desired in the light usage environment, in particular.

The detection device 17 has a function of obtaining the information on the ambient environment of the electronic device 10 or data on the user's emotion (hereinafter also referred to as user's data) and outputting the information or data to the arithmetic device 19. For example, the user data can be a temperature, a humidity, sound, an image, or the like. As the detection device 17, a temperature sensor, a humidity sensor, a microphone, or an imaging device can be used, for example. As the detection device, a still camera or video camera can be used, for example. Note that as the detection device 17, two or more of these devices may be used in combination.

The arithmetic device 19 has a function of processing arithmetically user's data output from the detection device 17 to generate display data in accordance with the user's feeing and outputting the display data to the display device 13.

As the arithmetic device 19, a CPU (Central Processing Unit), a DSP (Digital Signal Processor), a GPU (Graphics Processing Unit), or the like can be used. Furthermore, the arithmetic device 19 may be obtained with a PLD (Programmable Logic Device) such as a FPGA (Field Programmable Gate Array) or a FPAA (Field Programmable Analog Array).

The memory device 18 has a function of holding a program executed by the arithmetic device 19, data input to the arithmetic device 19, data output from the arithmetic device 19, and the like.

As the memory device 18, a memory device including a nonvolatile memory element can be preferably used. Examples of the memory device 18 include a flash memory, a MRAM (Magnetoresistive Random Access Memory), a PRAM (Phase change RAM), a ReRAM (Resistive RAM), and a FeRAM (Ferroelectric RAM).

FIG. 3B illustrates a structure different from that of the electronic device 10 in FIG. 3A.

The electronic device 10 illustrated in FIG. 3A includes an input/output device 21. The input/output device 21 has a function of obtaining information from the outside of the electronic device 10 and outputting information to the outside. The input/output device 21 has a function of obtaining information from the arithmetic device 19 and outputting information to the arithmetic device 19. Examples of the information obtained from the outside of the electronic device 10 include contents of video, music, game, and the like. The information output to the outside of the electronic device 10 is, for example, a user's emotion obtained by the electronic device 10.

For example, the input/output device 21 can be communicated with a wired or wireless network, and information can be input to and output from a server 23 via the network. Furthermore, in the case where a wireless communication is used for the network, besides near field communication means such as Wi-Fi (registered trademark) and Bluetooth (registered trademark), a variety of communication means such as the third generation mobile communication system (3G)-compatible communication means, LTE (sometimes also referred to as 3.9G)-compatible communication means, the fourth generation mobile communication system (4G)-compatible communication means, and the fifth generation mobile communication system (5G)-compatible communication means can be used.

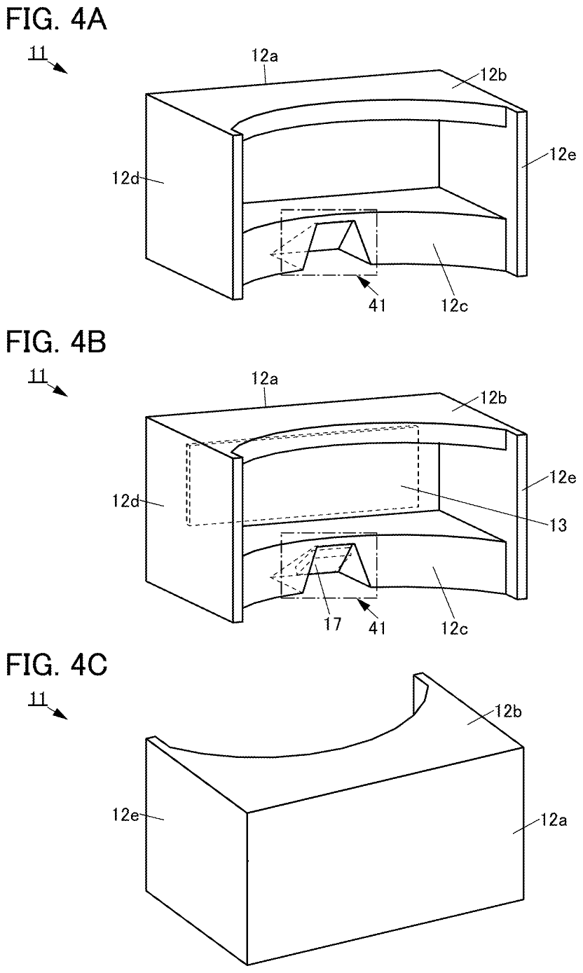

The housing 11 will be described with reference to FIG. 4A to FIG. 4C. FIG. 4A to FIG. 4C are external views illustrating the structure of the housing 11. In FIG. 4B, the display device 13 and the detection device 17 are illustrated with dashed lines to show the positional relationship between the display device 13, the detection device 17, and the housing 11.

The housing 11 includes a first portion 12a, a second portion 12b, a third portion 12c, a fourth portion 12d, and a fifth portion 12e. Note that FIG. 4A and FIG. 4B are perspective views on the side (on the user's side) opposite to the first portion 12a and FIG. 4C is a perspective view on the first portion 12a side (on the opposite side of the user).

The second portion 12b is connected to the first portion 12a. The third portion 12c is connected to the second portion 12b via the first portion 12a. The third portion 12c includes the space 41 indicated by a dashed-dotted line in FIG. 4A and FIG. 4B. The fourth portion 12d is connected to the first portion 12a, the second portion 12b, and the third portion 12c. The fifth portion 12e is connected to the first portion 12a, the second portion 12b, and the third portion 12c. The first portion 12a to the fifth portion 12e may be detachable from each other. Although a structure example in which the fourth portion 12d and the fifth portion 12e are not connected to each other is illustrated in FIG. 4A or the like, one embodiment of the present invention is not limited thereto. The fourth portion 12d and the fifth portion 12e may be connected to each other.

As illustrated in FIG. 4B, the display device 13 is positioned between the second portion 12b and the third portion 12c. The display device 13 may be fixed to any one or more of the first portion 12a and the fifth portion 12e. The detection device 17 is provided in the space 41 of the third portion 12c.

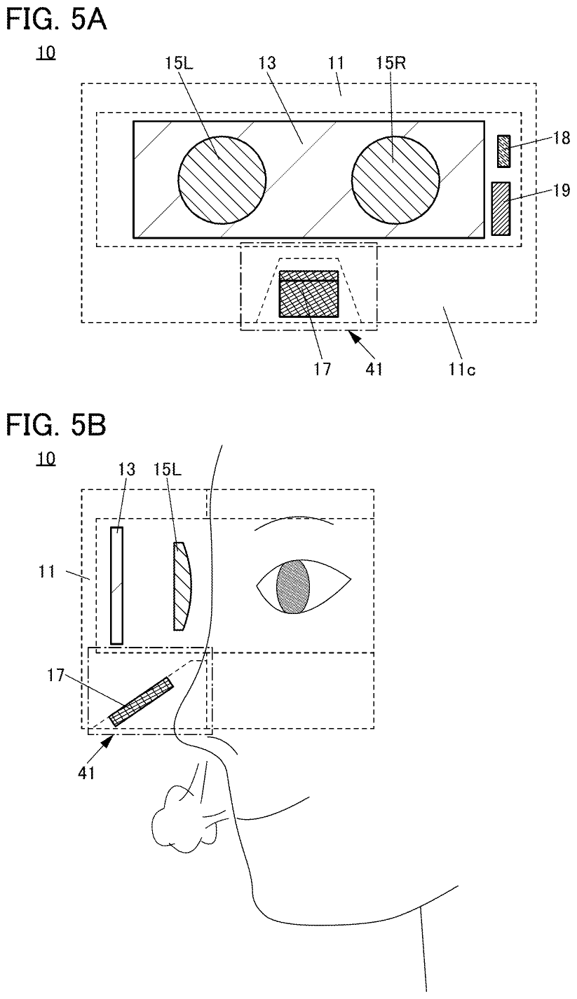

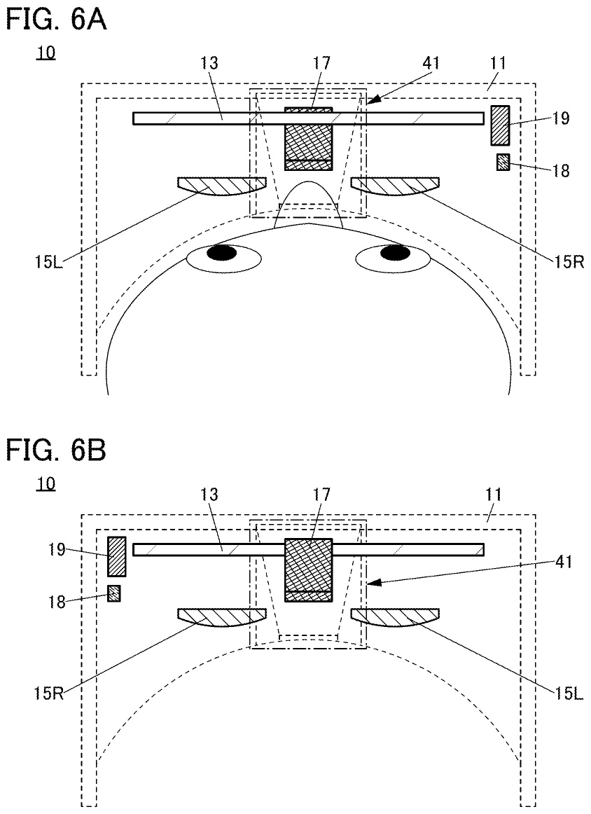

The positional relationship between the housing 11, the display device 13, the detection device 17, the arithmetic device 19, the memory device 18, the optical component 15L, and the optical component 15R is described with reference to FIG. 2A, FIG. 2B, FIG. 5A, FIG. 5B, FIG. 6A, and FIG. 6B.

FIG. 5A is an external view of the electronic device 10 that is seen from the side (the user's side) opposite to the first portion 12a. FIG. 5B is an external view of the electronic device 10 that is seen from the fourth portion 12d side (the left side of the user). FIG. 6A is an external view of the electronic device 10 that is seen from the second portion 12b side (the upper side of the user). FIG. 6B is an external view of the electronic device 10 that is seen from the third portion 12c (the lower side of the user). In FIG. 5A, FIG. 5B, FIG. 6A, and FIG. 6B, the first portion 12a, the second portion 12b, the third portion 12c, the fourth portion 12d, and the fifth portion 12e are shown by dashed lines. FIG. 5B and FIG. 6A illustrate an example of a state in which the electronic device 10 is worn by a user. In FIG. 5B, the detection device 17, the memory device 18, and the arithmetic device 19 are omitted for simplicity of the drawing.

The detection device 17 is preferably fixed to the third portion 12c. As illustrated in FIG. 5B and the like, preferably, the detection device 17 does not protrude from the housing 11. In the case where the detection device 17 protrudes from the housing 11, an interference between the user or another object and the detection device 17 might occur to cause a break in the detection device 17. When the detection device 17 is provided in the space 41 not to protrude from the housing 11, the detection device 17 can be prevented from being broken. Thus, the reliability of the electronic device 10 can be improved. In addition, the electronic device 10 can be downsized, and the convenience and the design can be improved.

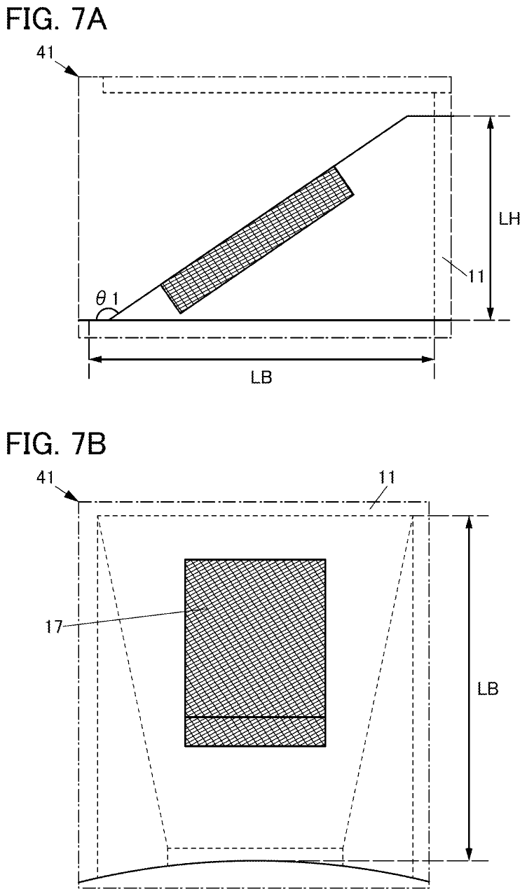

The space 41 is described. An enlarged view of the space 41 seen from the fourth portion 12d side (the left side of the user) is shown in FIG. 7A. FIG. 7B is an enlarged view of the space 41 seen from the third portion 12c side (the lower side of the user).

As illustrated in FIG. 5B, FIG. 7A, and FIG. 7B, the shape of the space 41 preferably has a width increasing toward the lower portion from the upper portion. The side surface of the space 41 preferably has such an angle that the detection device 17 can easily obtain information of the user's nose or mouth.

The angle θ1 of the angle between the housing 11 and the bottom portion of the space 41 is preferably larger than or equal to 120° and smaller than or equal to 170°, further preferably larger than or equal to 130° and smaller than or equal to 165°, further preferably larger than or equal to 135° and smaller than or equal to 160°, further preferably larger than or equal to 140° C. and smaller than or equal to 160°, still further preferably larger than or equal to 145° and smaller than or equal to 155°, yet still further preferably larger than or equal to 150° and smaller than or equal to 155°. The length LB of the space 41 is preferably greater than or equal to 30 mm and less than or equal to 100 mm, further preferably greater than or equal to 40 mm and less than or equal to 95 mm, further preferably greater than or equal to 50 mm and less than or equal to 90 mm, still further preferably greater than or equal to 60 mm and less than or equal to 85 mm, and yet still further preferably greater than or equal to 70 mm and less than or equal to 80 mm. The length LH of the space 41 is preferably greater than or equal to 30 mm and less than or equal to 100 mm, further preferably greater than or equal to 40 mm and less than or equal to 95 mm, further preferably greater than or equal to 50 mm and less than or equal to 90 mm, still further preferably greater than or equal to 60 mm and less than or equal to 85 mm, and yet still further preferably greater than or equal to 70 mm and less than or equal to 80 mm. When the space 41 has the above-described shape, the detection device 17 can be provided at a position not interfering with the user's nose. Furthermore, the detection device 17 can be provided at an angle that allows information of a user's nose or mouth to be easily obtained.

Preferably, the housing 11 does not cover a user's mouth as illustrated in FIG. 5B. A structure of the housing covering a use's mouth might make the user feel discomfort or oppression to the user. The electronic device 10 of one embodiment of the present invention can obtain information of a user's mouth, without the user's mouth covered by the housing 11.

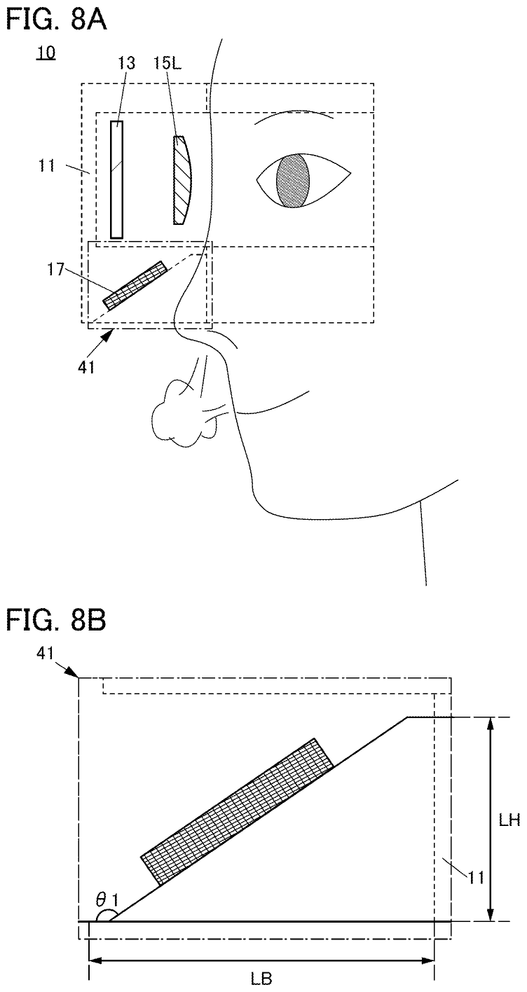

Although the drawings like FIG. 2A illustrate the structure in which the detection device 17 is positioned outside the housing 11, one embodiment of the present invention is not limited to this structure. As illustrated in FIG. 8A, the detection device 17 may be provided inside the housing 11. When the detection device 17 is provided inside the housing 11, the interference between the user and the detection device 17 can be prevented, leading to improved reliability of the electronic device. The housing 11 may include an opening (not illustrated) which is positioned between the detection device 17 and a user. When the housing 11 has the opening, the detection accuracy of the detection device 17 can be increased.

FIG. 8B is an enlarged view of the space 41. The angle θ1 of the angle between the housing 11 and the bottom portion of the space 41 is preferably within the above range.

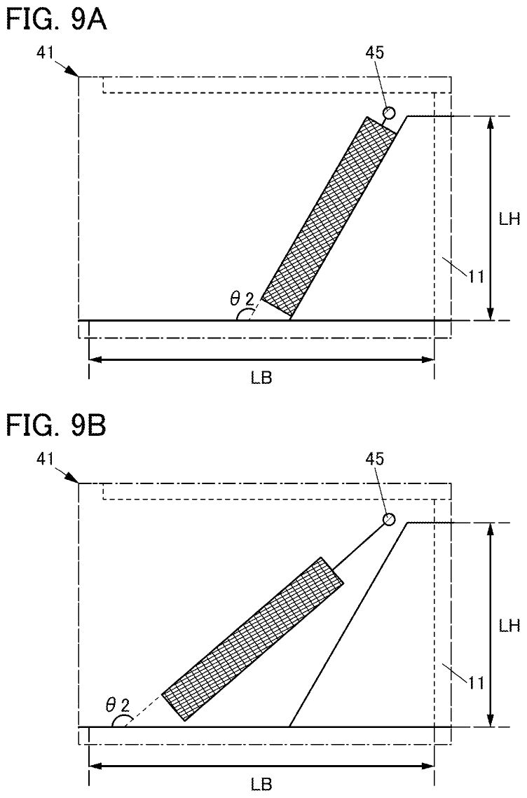

The electronic device 10 may include an adjustment mechanism for adjusting the position and the angle of the detection device 17. FIG. 9A and FIG. 9B illustrate a structure in which the electronic device 10 includes an adjustment mechanism 45. The adjustment mechanism 45 has a function of adjusting the position and the angle of the detection device 17 so as to match the state of each user's nose or mouth easily. The adjustment mechanism 45 is preferably fixed to the housing 11.

The angle θ2 between the housing 11 and the detection device 17 is preferably within the above-described range of the angle θ1. When the angle θ2 is within the above-described range, the detection device 17 can be provided at a position not interfering with the user's nose. Furthermore, the detection device 17 can be provided at an angle that allows information of a user's nose or mouth to be easily obtained. For example, when the angle 2θ is reduced, information on the user's nose can be easily obtained. For example, when the angle 2θ is increased, information of the user's mouth can be easily obtained.

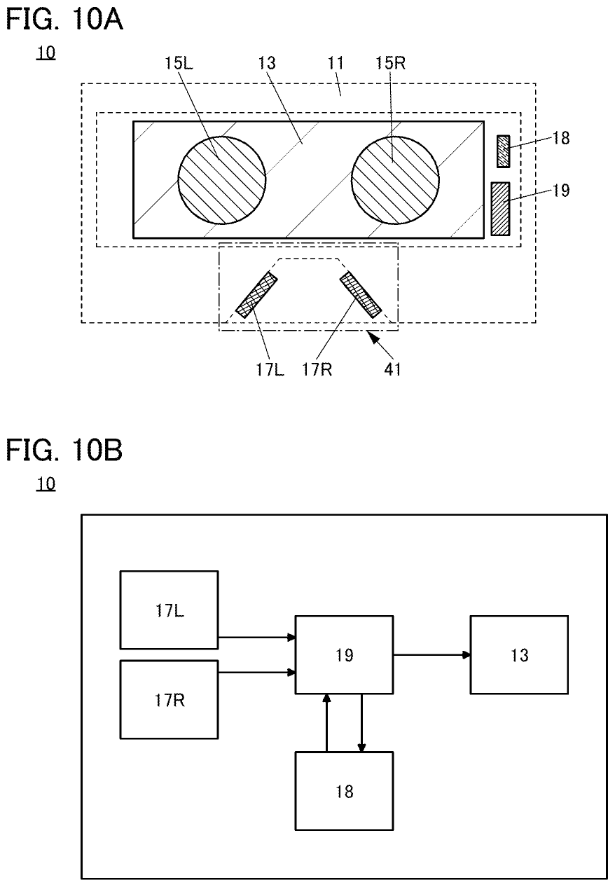

Although the drawings like FIG. 2A illustrate the structure in which one detection device 17 is provided in the space 41, one embodiment of the present invention is not limited to this structure. A plurality of detection devices 17 may be provided in the space 41. FIG. 10A is an external view of the electronic device 10 including a detection device 17L and a detection device 17R in the space 41. FIG. 10B is a block diagram illustrating the structure of the electronic device 10. FIG. 10A is an external view of the electronic device 10 seen from the side (the user side) opposite to the first portion 12a. Note that in FIG. 10A, the first portion 12a, the second portion 12b, the third portion 12c, the fourth portion 12d, and the fifth portion 12e are illustrated with dashed lines.

For example, in the space 41, the detection device 17L and the detection device 17R can be provided on the left side and the right side of the user, respectively. In that case, the detection device 17L and the detection device 17R can obtain, respectively, information on the left side and the right side of the user's nose. In addition, the angles of the detection device 17L and the detection device 17R may be adjusted so that the detection device 17L and the detection device 17R can obtain information on the right side and the left side of the user's mouth, respectively.

Data obtained by the detection device 17L and the detection device 17R are each output to the arithmetic device 19. With the use of the plurality of detection devices, the user's emotion can be obtained with a higher accuracy.

The arithmetic device 19 and the memory device 18 are positioned between the second portion 12b and the third portion 12c. The arithmetic device 19 and the memory device 18 may be fixed to one or more of the first portion 12a, the second portion 12b, the third portion 12c, the fourth portion 12d, and the fifth portion 12e. Although the example where the arithmetic device 19 and the memory device 18 are positioned on the fifth portion 12e side is illustrated in the drawings like FIG. 2A, one embodiment of the present invention is not limited to this example.

The optical component 15L and the optical component 15R are positioned between the second portion 12b and the third portion 12c. The optical component 15L and the optical component 15R may be each fixed to one or more of the first portion 12a, the second portion 12b, the third portion 12c, the fourth portion 12d, and the fifth portion 12e.

As illustrated in FIG. 5B and the like, the detection device 17 is provided in the space 41. While the electronic device 10 is used, the user's nose is positioned in the space 41. The shape of the space 41 preferably has a width increasing toward the lower portion from the upper portion. In other words, the space 41 preferably has such a shape that the width increases from the second portion 12 side toward the third portion 12c side. When the space 41 has such a shape, the detection device 17 provided in the space 41 can easily obtain information of the surroundings of the user's nose or mouth.

When the user gets excited in using the electronic device 10, the temperatures of the nasal breath and breath are sometimes increased with an increase of the body temperature, leading to the increase of the temperature of the surrounding area of the user's nose and mouth. A temperature sensor is used as the detection device 17 to obtain the temperature of the surrounding area of the user's nose or mouth, whereby the degree of the user's excitement can be estimated. For example, it can be estimated that the higher the temperature of the surrounding area of the user's nose or mouth is, the higher the degree of the user's excitement is.

When the user gets excited in using the electronic device 10, the skin temperature of a user's nose or mouth is sometimes increased with an increase of the body temperature. A temperature sensor is used as the detection device 17 to obtain the skin temperature around the user's nose and mouth, whereby the degree of the user's excitement can be estimated. For example, it can be estimated that the higher the skin temperature around the user's nose and mouth is, the higher the degree of the user's excitement is.

When the user gets excited in using the electronic device 10, breathing becomes faster and the humidity of the surrounding area of the user's nose or mouth is sometimes increased by the nasal breath and breath. A humidity sensor is used as the detection device 17 to obtain the humidity of the surrounding area of the user's nose and mouth, whereby the degree of the user's excitement can be estimated. For example, it can be estimated that the higher the humidity of the surrounding area of the user's nose and mouth is, the higher the degree of the user's excitement is.

When the user gets excited in using the electronic device 10, the voice becomes sometimes loud. A microphone is used as the detection device 17 to obtain the voice of the user, whereby the degree of the user's excitement can be estimated. For example, it can be estimated that the larger the volume of the user's voice is, the higher the degree of the user's excitement is.

When the user gets excited in using the electronic device 10, the user sometimes sweats with an increase of the body temperature. An imaging device is used as the detection device 17 to take an image of the user's nose or a portion below the nose and obtain the sweating state of the nose or the portion below the nose, whereby the degree of the user's excitement can be estimated. For example, it can be estimated that the larger the sweat amount of the nose or the portion below the nose is, the higher the degree of the user's excitement is.

While the user is using the electronic device 10, the kind or the degree of the user's emotion may change. An image of the user's mouth is captured by using an imaging device as the detection device 17 to obtain the shape of the mouth, whereby the kind or the degree of the user's emotion can be estimated.

In the case where an imaging device is used as the detection device 17, the detection device 17 may include a light source (not illustrated). With the light source, light emitted from the light source can be reflected by the user's face and the reflected light can be detected by the detection device 17. For example, the light source preferably has a function of emitting red light, and the imaging device preferably has a function of detecting red light. For example, the light source preferably has a function of emitting near-infrared light, and the imaging device preferably has a function of detecting near-infrared light. For example, a light source preferably has a function of emitting mid-infrared light, and the imaging device preferably has a function of detecting mid-red light. For example, the light source preferably has a function of emitting far-infrared light, and the imaging device preferably has a function of detecting far-infrared light.

Thus, the electronic device 10 can obtain the sweating state of the user or the shape of the user's mouth with a high accuracy.

In this specification and the like, infrared light refers to light with a wavelength ranging from 0.7 μm to 1000 μm, inclusive, for example. Near-infrared light refers to light with a wavelength ranging from 0.7 μm to 2.5 μm, inclusive, for example. Mid-infrared light refers to light with a wavelength ranging from 2.5 μm to 4 μm, inclusive, for example. Far-infrared light refers to light with a wavelength ranging from 4 μm to 1000 μm, inclusive, for example. Note that near-infrared light, mid-infrared light, or far-infrared light may be simply referred to as infrared light. In this specification and the like, red light refers to light with a wavelength ranging from 0.6 μm to 0.75 μm, inclusive, for example.

The electronic device 10 of one embodiment of the present invention can obtain the degree of a user's excitement and the kind or the degree of a user's emotion by having the detection device 17. Note that in this specification and the like, the degree of a user's excitement and the kind or the degree of a user's emotion are collectively referred to as a user's emotion. In addition, the electronic device 10 of one embodiment of the present invention can display information corresponding to the user's emotion on the display device 13. A character (also referred to as an avatar) as an alter ego of the user can have a facial expression corresponding to the user's emotion, which can be displayed on the display device 13. The user can know his/her emotion and have a higher sense of immersion. In addition, the user can know his/her emotion and decide to have a break or the like.

As illustrated in FIG. 5B, the detection device 17 can be positioned outside the housing 11. In that case, the detection device 17 is positioned between the housing 11 and the user's nose. The detection device 17 is positioned outside the housing 11; thus, the detection accuracy of the detection device 17 can be increased because the housing 11 is not positioned between the user and the detection device 17.

A region on the side opposite to the first portion 12a of the second portion 12b is a portion in contact with a user's forehead. A region on the side opposite to the first portion 12a of the third portion 12c is a portion in contact with a user's cheek. The regions preferably each have a curved shape, specifically, a circular arc shape toward the first portion 12a. With the regions each having a curved shape or a circular arc shape, the second portion 12b can be closely contact with the user's cheek or forehead. Thus, light leakage from the outside of the electronic device 10 is suppressed and the user can feel a higher sense of immersion. The shape of the housing 11 of the electronic device 10 is not limited to the structure illustrated in the drawings like FIG. 2A.

The optical component 15L and the optical component 15R each include a region overlapping with the display device 13, and are positioned between the display device 13 and the user. The user can view an image displayed on the display device 13 through the optical component 15L and the optical component 15R. The drawings like FIG. 2A illustrate the optical component 15L for the left eye and the optical component 15R for the right eye. The optical component 15L and the optical component 15R each have a function of expanding and projecting an image displayed on the display device 13 for a user. For example, convex lenses can be used as the optical component 15L and the optical component 15R. The optical component 15L and the optical component 15R are each one convex lens in the drawings like FIG. 2A; however, there is no particular limitation on the shape and a plurality of optical components may be used.

For example, plastic or glass can be used as the materials of the optical component 15L and the optical component 15R. For plastic, a material with a high visible-light transmitting property is preferable, for example, a urethane resin, an acrylic resin, a carbon resin, an allylic resin, or the like can be used. In addition, when a material in which a halogen, an aromatic ring, or sulfur with a high atomic refraction is added to such plastics is used, the refractive indices of the optical component 15L and the optical component 15R can be increased. As the halogen, any one or more of chlorine, bromine, and iodine is preferably selected.

### Structure Example 2 of Electronic Device

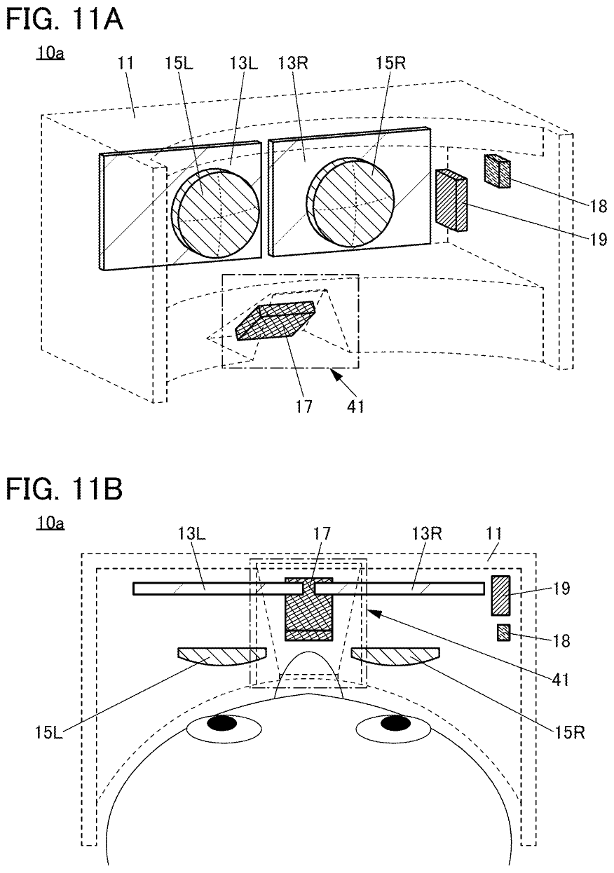

Although the drawings like FIG. 2A illustrate the structural example in which the electronic device 10 includes one display device, one embodiment of the present invention is not limited to this example. An electronic device of one embodiment of the present invention may include a plurality of display devices. FIG. 11A and FIG. 11B illustrate a structural example of an electronic device 10a including two display devices. FIG. 11A is a perspective view illustrating the appearance of the electronic device 10a. FIG. 11B is an external view of the electronic device 10a seen from the second portion 12b side. In FIG. 11A and FIG. 11B, the housing 11 is illustrated with a dashed line. FIG. 11B illustrates an example in which a user wears the electronic device 10a.

The electronic device 10a illustrated in FIG. 11A and FIG. 11B includes a display device 13L and a display device 13R. With the display device 13L and the display device 13R included in the electronic device 10a, user's eyes can view images displayed on the respective display devices. This allows a high-definition image to be displayed even when three-dimensional display using parallax or the like is performed.

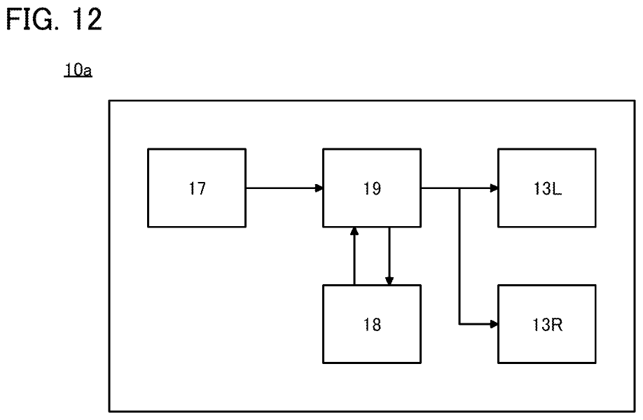

FIG. 12 is a block diagram illustrating a structure example of the electronic device 10a. Display data generated by the arithmetic device 19 is output to the display device 13L and the display device 13R as different data.

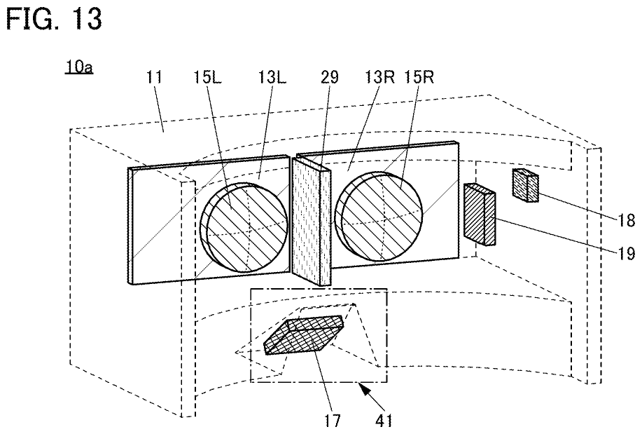

As illustrated in FIG. 13, the electronic device 10a may further include a separator 29. The separator 29 is preferably provided to be orthogonal to the display surfaces of the display device 13L and the display device 13R. The separator 29 is preferably provided to be closer to the user than the display device 13L and the display device 13R are. The separator 29 can separate the field of view of the right eye from the left eye, so that the right and left eyes perceive respective images different from each other, which can cause binocular parallax. The binocular parallax allows a user to see an image three-dimensionally.

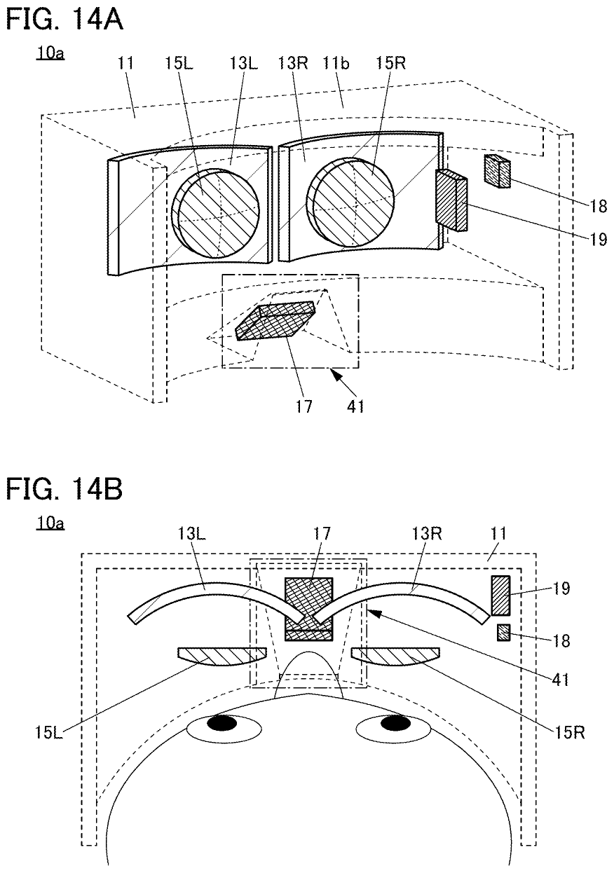

Although the drawings like FIG. 2A illustrate the structural example in which the display device included in the electronic device is planar, one embodiment of the present invention is not limited to this example. The display device included in the electronic device of one embodiment of the present invention may be curved. FIG. 14A and FIG. 14B illustrate a structural example of an electronic device 10b including a curved display device. FIG. 14A is a perspective view illustrating an appearance of the electronic device 10b. FIG. 14B is an external view of the electronic device 10b viewed from the second portion 12b side. In FIG. 14A and FIG. 14B, the housing 11 is illustrated with a dashed line. FIG. 14B illustrates an example in which a user wears the electronic device 10b.

In FIG. 14A and FIG. 14B, each of the display device 13L and the display device 13R has a shape of a circular arc with the user's eye as a substantially center. This keeps a certain distance between the user's eye and the display surface of the display portion, enabling the user to see a more natural image. Even when the luminance or chromaticity of light from the display portion is changed depending on the angle at which the user see it, since the user's eye is positioned in a normal direction of the display surface of the display portion, the influence of the change can be substantially ignorable and thus a more realistic image can be displayed.

### Structure Example 3 of Electronic Device

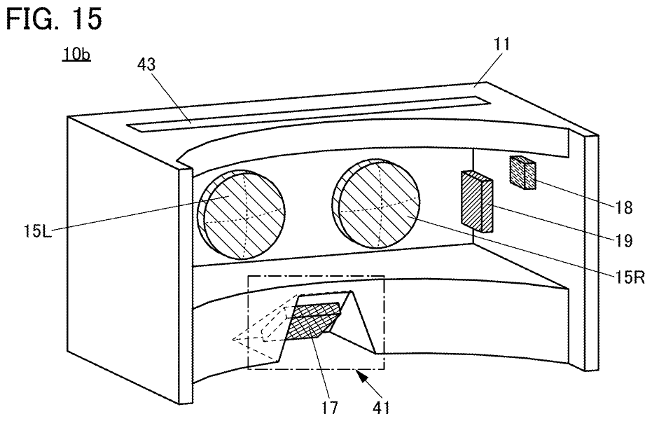

FIG. 15 illustrates a structure example different from those of the electronic device 10 and the electronic device 10a describe above. FIG. 15 is a perspective view illustrating the appearance of the electronic device 10b.

As illustrated in FIG. 15, the electronic device 10b includes the housing 11, the detection device 17, the arithmetic device 19, and the memory device 18. The housing 11 includes the space 41 in a lower portion, and the detection device 17 is provided in the space 41. The electronic device 10b is different from the electronic device 10 and the electronic device 10a mainly in that the display device 13 is not included. The electronic device 10b may further include the optical component 15L and the optical component 15R.

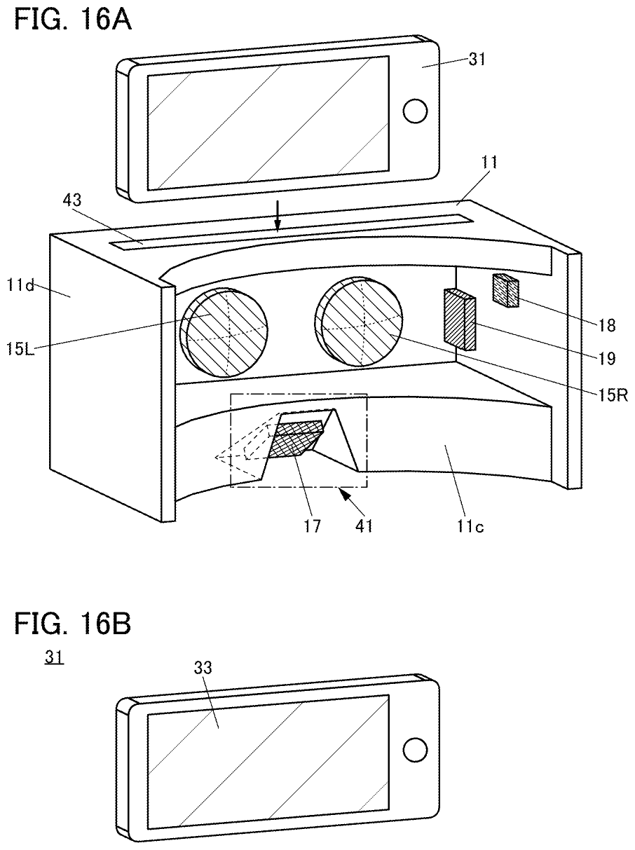

As illustrated in FIG. 16A and FIG. 16B, the electronic device 10b can be combined with another electronic device with a display portion. Examples of such another electronic device include a smartphone and a portable game machine. Such another electronic device is connected to the arithmetic device 19 through a connector (not illustrated) included in the electronic device 10b.

FIG. 16A and FIG. 16B illustrate a structure example of a smartphone that can be used as an electronic device 31. The electronic device 31 includes a display portion 33 and is attached to the inside of the electronic device 10b through an opening portion 43, whereby the electronic device 31 can function in a manner similar to the display device 13 in FIG. 2A.

Note that although FIG. 15 and FIG. 16A illustrate the structure in which the second portion 12b includes the opening portion 43, one embodiment of the present invention is not limited to this structure. The opening portion 43 may be provided in one or more of the first portion 12a to the fifth portion 12e. The electronic device 10b does not necessarily include the opening portion 43. By making a portion of the electronic device 10b detachable, the electronic device 31 can be set inside the electronic device 10b by detaching the portion. For example, when the first portion 12a is detachable, the user can detach the first portion 12a and set the electronic device 31 to be set inside the electronic device 10b. Without providing the opening portion 43, external light can be prevented from entering the electronic device 10b.

<Estimation of User's Emotion>

A method for estimating a user's emotion is described. Here, an example of an image of a portion including a user's mouth will be described.

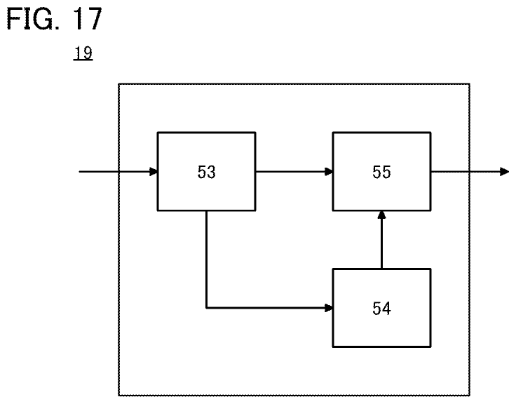

FIG. 17 is a block diagram illustrating a structure example of the arithmetic device 19. The arithmetic device 19 includes a feature-extraction unit 53, an estimation unit 54, and an information-generation unit 55.

The feature-extraction unit 53 has a function of extracting feature points from the image of a portion including a user's mouth output from the detection device 17, obtaining a feature value calculated from the positions of feature points, and outputting the feature value to the estimation unit 54.

In the case where information obtained by the detection device 17 is an image of the portion including the user's mouth, the feature points are, for example, an upper edge of the upper lip, a lower edge of the lower lip, the right corner and the left corner of the mouth.

As a method of feature-extraction by the feature-extraction unit 53, various types of algorithm can be employed. For example, an algorithm such as SIFT (Scaled Invariant Feature Transform), SURF (Speeded Up Robust Features), or HOG (Histograms of Oriented Gradients) can be used in the feature-extraction unit 53.

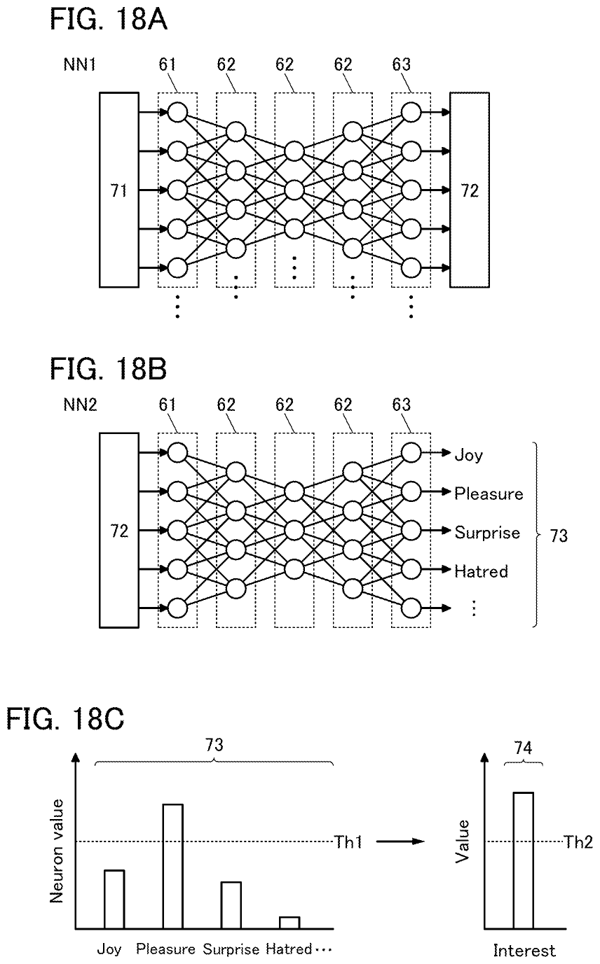

For the feature-extraction by the feature-extraction unit 53, a neural network can be used. FIG. 18A schematically illustrates a neural network NN1 that can be used in the feature-extraction unit 53. The neural network NN1 includes an input layer 61, three intermediate layers 62, and an output layer 63. Although FIG. 18A illustrates the structure in which the feature-extraction unit 53 includes three intermediate layers 62, one embodiment of the present invention is not limited thereto. The feature-extraction unit 53 may one or more intermediate layers 62.

Data 71 is input to the neural network NN1. Image data captured by the detection device 17 can be used as the data 71, for example. The data 71 includes coordinates and gray-scale values of each pixel. Data 72 is output from the neural network NN1. The data 72 includes the position coordinates of the aforementioned feature point.

The neural network NN1 has learned so as to extract the aforementioned feature point from the data 71 such as image data and output the coordinates of the feature point. The neural network NN1 has learned so that edge computing using various filters or the like in the intermediate layers 62 increases a neuron value of the output layer 63 corresponding to the coordinates of the aforementioned feature point.

The estimation unit 54 has a function of estimating the user's emotion of the electronic device 10 from the information of the feature point, which is input from the feature-extraction unit 53, and outputting the estimated information to the information-generation unit 55. A neural network can be used for the estimation by the estimation unit 54.

FIG. 18B schematically illustrates a neural network NN2 that can be used in the estimation unit 54. FIG. 18B illustrates the case where the estimation unit 54 estimates the user's emotion of the electronic device 10. An example where the neural network NN2 has substantially the same structure as the neural network NN1 is illustrated here. Note that the number of neurons of the input layer 61 in the neural network NN2 can be smaller than that in the neural network NN1.

The data 72 generated by the feature-extraction unit 53 is input to the neural network NN2. The data 72 includes information on the coordinates of the extracted feature point.

As data input to the neural network NN2, data obtained by processing the data 72 may be used. For example, data obtained by performing calculation of a vector connecting given two feature points on all or some of the feature points may be used as data input to the neural network NN2. Moreover, data obtained by normalizing the calculated vectors may be used. Note that hereinafter, data obtained by processing the data 72 output from the neural network NN1 is also referred to as the data 72.

Data 73 is output from the neural network NN2 to which the data 72 is input. The data 73 corresponds to neuron values output from respective neurons of the output layer 63. Each neuron of the output layer 63 is associated with one emotion. As illustrated in FIG. 18B, the data 73 includes neuron values of the neurons each corresponding to a predetermined emotion (e.g., joy, pleasure, surprise, and hatred).

The neural network NN2 has learned so as to estimate the degree of each emotion from the data 72 and output the estimation as neuron values. Accordingly, the user's emotion can be estimated from the shape of the user's mouth by the neural network NN2.

FIG. 18C schematically illustrates the data 73. The level of a neuron value corresponding to each emotion indicates the level of the degree of an estimated emotion. In addition, the estimation unit 54 may estimate the degree of a different emotion from the estimated degree of the emotion. Data including the degree of another emotion is referred to as data 74. FIG. 18C illustrates the case where the degree of interest is estimated from the degrees of emotions such as joy, pleasure, surprise, and hatred.

The degree of interest, which is included in the data 74, can be estimated, for example, by inputting the degrees of emotions such as joy, pleasure, surprise, and hatred, which are included in the data 73, to a predetermined formula. For example, the formula can be set so that the degree of interest increases as the degrees of joy, pleasure, and surprise are higher and the degree of interest decreases as the degree of hatred is higher.

Note that the estimation of emotions can also be estimated without using a neural network. For example, a pattern matching method, a template matching method, or the like in which the image of a portion including the user's mouth obtained by the detection device 17 is compared with a template image and the similarity degree is used, may be employed. In that case, a structure without the feature-extraction unit 53 can also be employed.

The information-generation unit 55 has a function of determining or generating information to be shown to the user on the basis of the emotion, which is estimated by the estimation unit 54, and outputting the information to the display device 13. Accordingly, the display device 13 can show information corresponding to the information generated in the information-generation unit 55.

Note that the data 72 output from the feature-extraction unit 53 may be directly input to the information-generation unit 55 without being input to the estimation unit 54. For example, the user's emotion can be detected by extraction of feature points with the feature-extraction unit 53 without estimation by the estimation unit 54. In such a case, the data 72 output from the feature-extraction unit 53 is directly input to the information-generation unit 55, whereby power consumption of the electronic device 10 can be reduced.

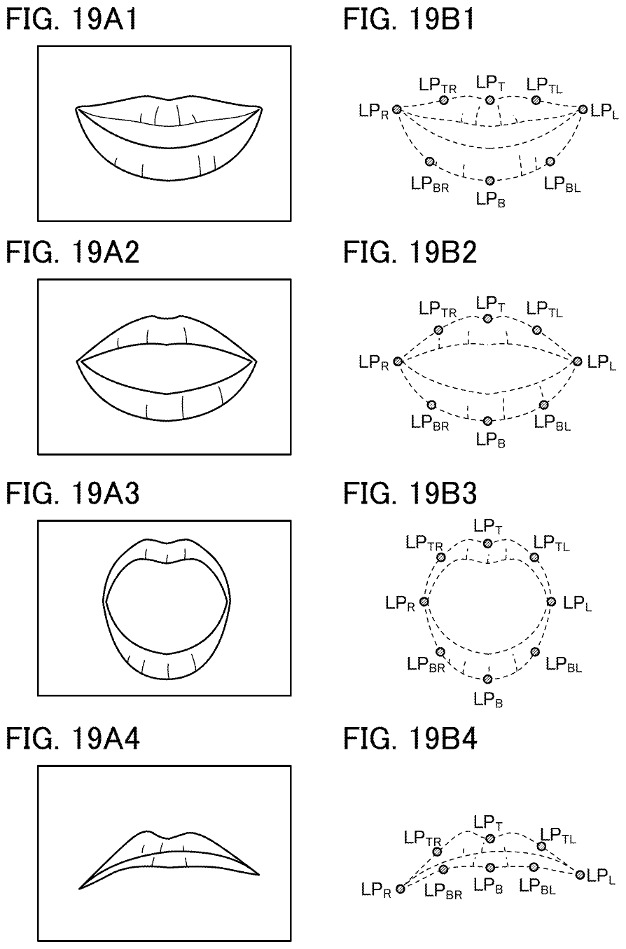

FIG. 19A1 to FIG. 19A4 illustrate examples of images of the portions including the user's mouth which can be used as the data 71. FIG. 19A1 to FIG. 19A4 illustrate examples of images of the portions including the user's mouth with a high degree of the user's emotion “joy”, a high degree of “pleasure”, a high degree of “surprise”, and a high degree of “hatred”. FIG. 19B1 to FIG. 19B4 illustrate examples in which feature points of the images of the portions including the mouth illustrated in FIG. 19A1 to FIG. 19A4 are extracted. In examples illustrated in FIG. 19B1 to FIG. 19B4, upper edges of the upper lip, LP_{TL}, LP_{T}, and LP_{TR}; the lower edges of the lower lip, LP_{BL}, LP_{B}, and LP_{BR}, the right corner of the mouth, LP_{R}; and the left corner of the mouth, LPL are extracted as the feature points. The feature-extraction unit 53 extracts these feature points and output the information of the feature points to the estimation unit 54. The estimation unit 54 estimates the user's emotion on the basis of the information of the feature points and outputs the information of the user's emotion to the information-generation unit 55. The information-generation unit determines or generates information to be shown to the user from the information of the user's emotion and outputs the information to be shown to the user to the display device 13. The display device 13 can display the information to be shown to the user.

### Example of Information to be Shown to the User

An example of user's information to be shown in the electronic device is described below.

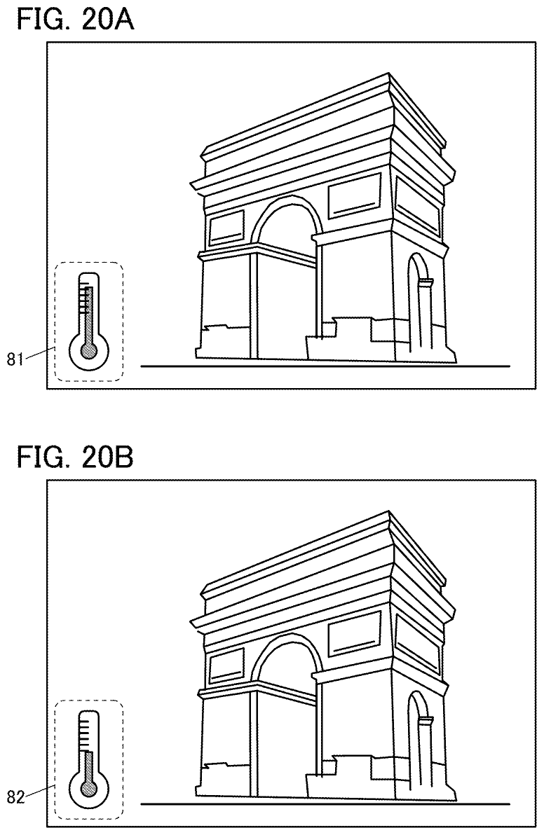

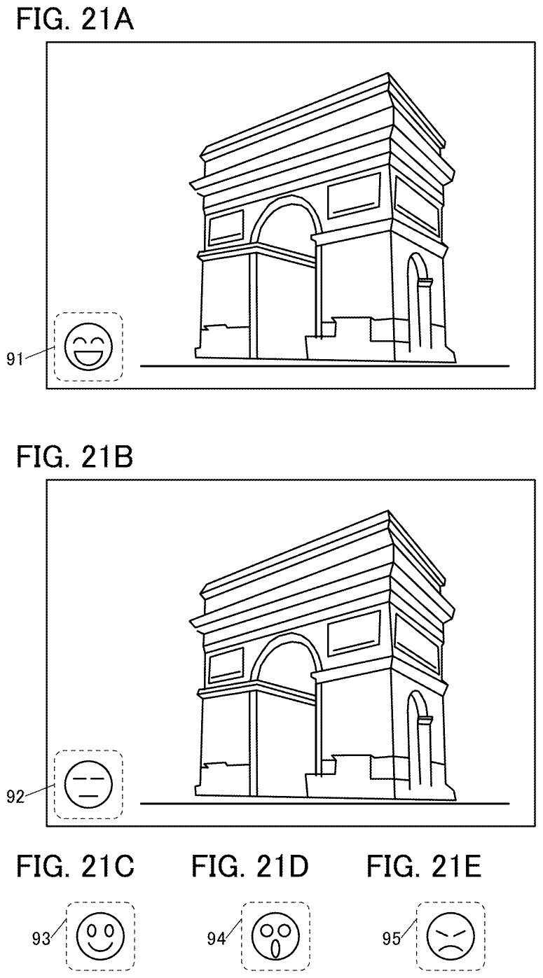

FIG. 20A, FIG. 20B, FIG. 21A, and FIG. 21B illustrate examples of a user's field of view when the user uses an electronic device of one embodiment of the present invention. FIG. 20A, FIG. 20B, FIG. 21A, and FIG. 21B illustrate examples of a user's field of view when the user views an image of a tourism destination.

In FIG. 20A and FIG. 20B, information 81 and information 82 representing the degree of the use's excitement are shown on the left lower part of the field of view, superimposed on the displayed image.

The information 81 illustrated in FIG. 20A is an example in which the degree of the user's excitement is judged to be high on the basis of the user's data. For example, when the temperature of the surrounding area of the user's nose or mouth is higher than or equal to a predetermined temperature, the skin temperature around the user's nose or mouth is higher than or equal to a predetermined temperature, the humidity of the surrounding area of the user's nose or mouth is higher than or equal to a predetermined humidity, the volume of a voice is higher than or equal to a predetermined volume, or the sweating amount of the nose or below the nose is higher than or equal to a predetermined amount, the degree of the user's excitement is judged to be high.

The user's data may be shown as the information 81. For example, the temperature of the surrounding area of the user's nose or mouth can be shown as the information 81. For example, the volume of a voice can be shown as the information 81.

The information 82 illustrated in FIG. 20B is an example in which the degree of the user's excitement is judged to be low on the basis of the user's data. For example, when the temperature of the surrounding area of the user's nose or mouth is lower than a predetermined temperature, the skin temperature around the user's nose or mouth is lower than a predetermined temperature, the humidity of the surrounding area of the user's nose or mouth is lower than a humidity, the volume of a voice is lower than a predetermined volume, or the sweating amount of the nose or below the nose is lower than a predetermined amount, the degree of the user's excitement is judged to be low.

As described above, the user can know the degree of his/her excitement by showing the degree of the user's excitement to the user, and have a higher sense of immersion. Alternatively, the user can know the degree of his/her excitement and can choose to take a break or the like.

In FIG. 21A and FIG. 21B, information 91 and information 92 represented by a character modeling the user's emotion are shown on the left lower part of the field of view, superimposed on the displayed image.

FIG. 21A illustrates an example in which the degree of the user's emotion “pleasure” is judged to be high. For example, this corresponds to the case in which the neuron value of “pleasure” is the highest value in FIG. 18C. In addition, FIG. 21A illustrates an example in which the degree of the user's interest is judged to be high. For example, FIG. 21A corresponds to the case in which the value exceeds the threshold value Th2 in FIG. 18C.

FIG. 21B illustrates an example in which the user's emotions, “joy”, “pleasure”, “surprised”, and “hatred” are all judged to be low. For example, this corresponds to a case in which none of the neuron values exceeds the threshold value Th1 in FIG. 18C. In addition, FIG. 21B illustrates an example in which the degree of the user's interest is judged to be low. For example, FIG. 21B corresponds to the case in which the value is below a threshold value Th2 in FIG. 18C.

In FIG. 18C, when the degree of the user's emotion “joy” is judged to be high, information 93 illustrated in FIG. 21C can be shown, for example. When the degree of the user's emotion “surprise” is judged to be high, information 94 illustrated in FIG. 21D can be shown, for example. When the degree of the user's emotion “hatred” is judged to be high, information 95 illustrated in FIG. 21E can be shown, for example.

Although FIG. 21A and FIG. 21B each illustrate an example in which one piece of information is shown, one embodiment of the present invention is not limited thereto. A plurality pieces of information may be shown, superimposed on the displayed image. For example, in FIG. 18C, when the neuron values of “happiness” and “surprise” exceed the threshold value Th1, information each corresponding to them can be shown. For example, one piece or more pieces of information 91 and information 94 can be shown.

As described above, a character having a facial expression reflecting the emotion in accordance with the estimated user's emotion is shown to the user, whereby the user can know his/her emotion and have a higher sense of immersion. Alternatively, the user can know an emotion that he/she does not perceive. The method of showing the information of the emotion to the user using a facial expression of a character is described here, but without being limited to this, various methods can be used as long as the kind or degree of emotions can be visualized.

### Structure Example of Housing

In the electronic device of one embodiment of the present invention, the housing 11 includes the space 41 positioned at the user's nose, and there is no particular limitation on the structure and the shapes of the portions other than the space 41.

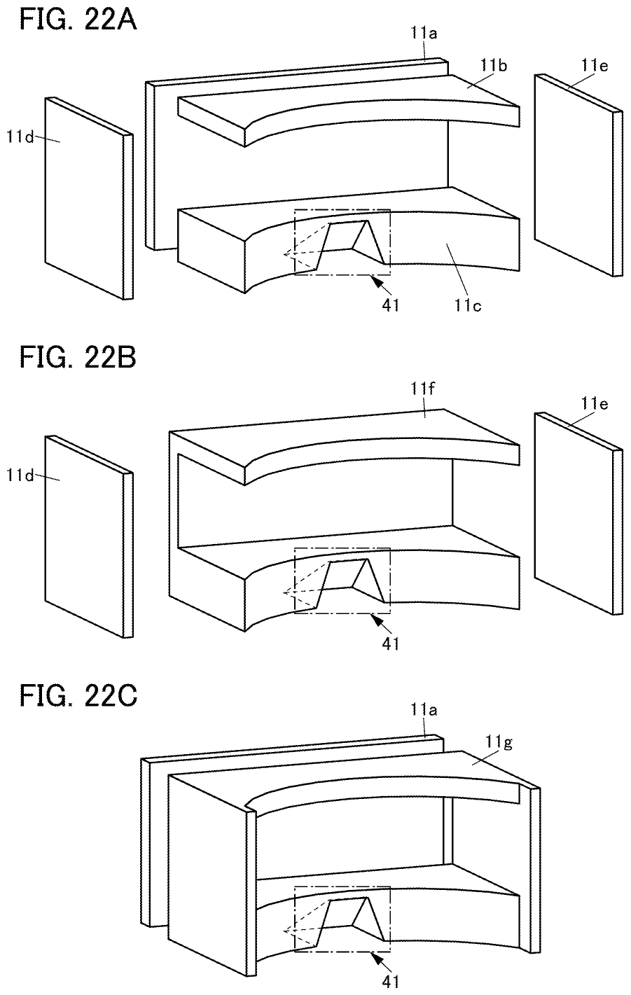

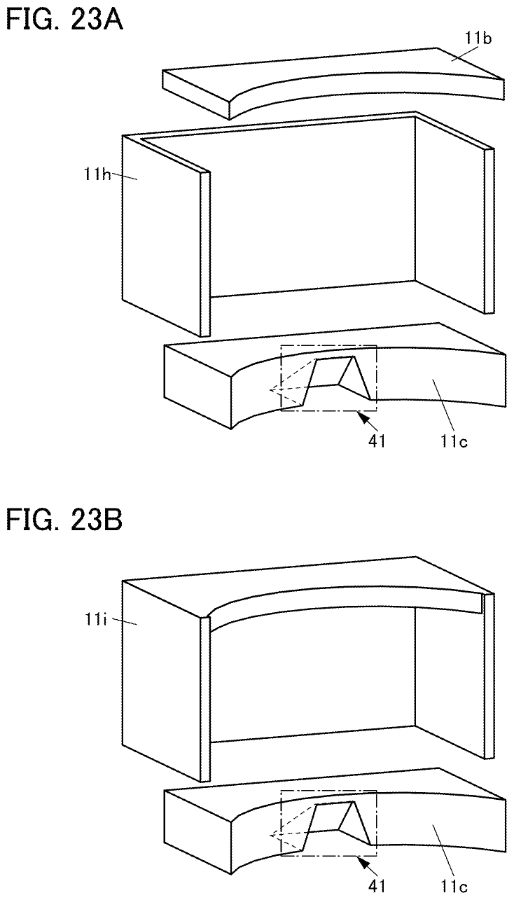

The housing 11 can have a structure in which a plurality of housings are connected. Examples of the structure of the housing 11 are illustrated in FIG. 22A, FIG. 22B, FIG. 22C, FIG. 23A, and FIG. 23B.

FIG. 22A illustrates an example in which the housing 11 includes a first part 11a to a fifth part 11e. In the structure illustrated in FIG. 22A, the first portion 12a to the fifth portion 12e illustrated in FIG. 4A to FIG. 4C correspond to the first part 11a to the fifth part 11e, respectively. The first part 11a to the fifth part 11e are connected to each other to form the housing 11. In addition, any one or more of the first part 11a to the fifth part 11e may be detachable from the housing 11.

FIG. 22B illustrates an example in which the housing 11 includes the fourth part 11d, the fifth part 11e, and a sixth part 11f. In the structure illustrated in FIG. 22B, the first portion 12a to the third portion 12c illustrated in FIG. 4A to FIG. 4C are unified to be the sixth part 11f. The fourth part 11d, the fifth part 11e, and the sixth part 11f are connected to each other to form the housing 11. In addition, any one or more of the fourth part 11d, the fifth part 11e, and the sixth part 11f may be detachable from the housing 11.

FIG. 22C illustrates an example in which the housing 11 includes the first part 11a and a seventh part 11g. In the structure illustrated in FIG. 22C, the second portion 12b to the fifth portion 12e illustrated in FIG. 4A to FIG. 4C are unified to be the seventh part 11g. The first part 11a and the seventh part 11g are connected to each other to form the housing 11. In addition, either the first part 11a or the seventh part 11g can be detachable from the housing 11.

FIG. 23A illustrates an example in which the housing 11 includes the second part 11b, the third part 11c, and an eighth part 11h. In the structure illustrated in FIG. 23A, the first portion 12a, the fourth portion 12d, and the fifth portion 12e illustrated in FIG. 4A to FIG. 4C are unified to be the eighth part 11h. The second part 11b, the third part 11c, and the eighth part 11h are connected to each other to form the housing 11. In addition, any one or more of the second part 11b, the third part 11c, and the eighth part 11h can be detachable from the housing 11.

FIG. 23B illustrates an example in which the housing 11 includes the third part 11c and a ninth part 11i. In the structure illustrated in FIG. 23B, the first portion 12a, the second portion 12b, the fourth portion 12d, and the fifth portion 12e illustrated in FIG. 4A to FIG. 4C are unified to be the seventh part 11g. The third part 11c and the ninth part 11i are connected to each other to form the housing 11. In addition, either the third part 11c or the ninth part 11i can be detachable from the housing 11.

In FIG. 22A to FIG. 22C, FIG. 23A, and FIG. 23B, separated parts are illustrated for simplification of the drawings.

By connecting a plurality of parts to form the housing 11, it becomes easy to perform loading a component (such as the arithmetic device 19) provided in the electronic device 10. For example, in the housing 11 including the first part 11a to the fifth part 11e, a component is loaded to each of the first part 11a to the fifth part 11e and then the first part 11a to the fifth part 11e can be connected. This case can increase the productivity, as compared with the productivity in the case of loading the components after the first part 11a to the fifth part 11e are connected. Furthermore, one or some of the parts can be detachable from the housing 11, whereby a broken component can be easily replaced, for example.

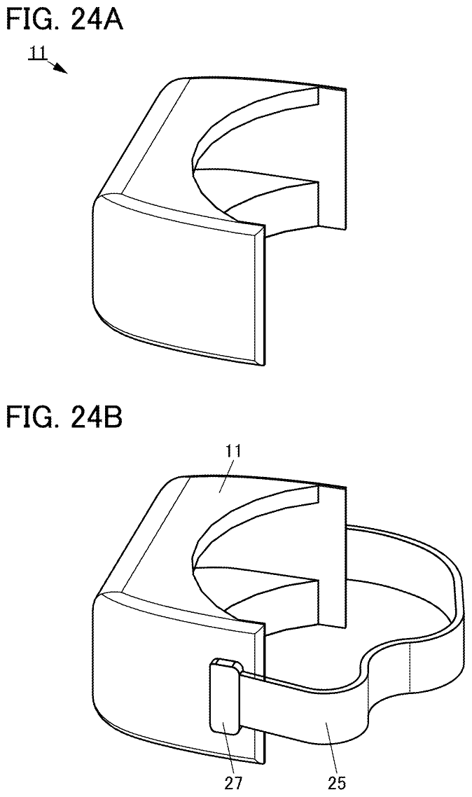

The shape of the housing 11 is not particularly limited to the shape shown in the drawings like FIG. 2A. Each portion included in the housing 11 may have a curved surface. An example of the housing 11 having a curved surface is illustrated in FIG. 24A. When the housing 11 has a curved surface, the design of the electronic device of one embodiment of the present invention can be increased. When the housing 11 has a curved surface and the corner portion is reduced, the user can be prevented from getting injured, even if he/she comes in contact with the housing, which can increase the safety of the electronic device of one embodiment of the present invention.

The electronic device of one embodiment of the present invention may include a fastening 25 as illustrated in FIG. 24B. With the fastening 25, the housing 11 can be fixed to the user's head. Note that although the fastening 25 in FIG. 24B has a band shape, one embodiment of the present invention is not limited thereto. Although the end of the fastening 25 is fixed to the housing 11 with a buckle catch 27 in FIG. 24B, another structure may be employed. For example, a structure without the buckle catch 27 may be employed.

At least part of this embodiment can be implemented in combination with any of the other embodiments described in this specification, as appropriate.

### Embodiment 2

In this embodiment, a display device, a light source, an imaging device, and the like that can be used in the electronic device of one embodiment of the present invention will be described.

### Structure Example 1 of Display Device

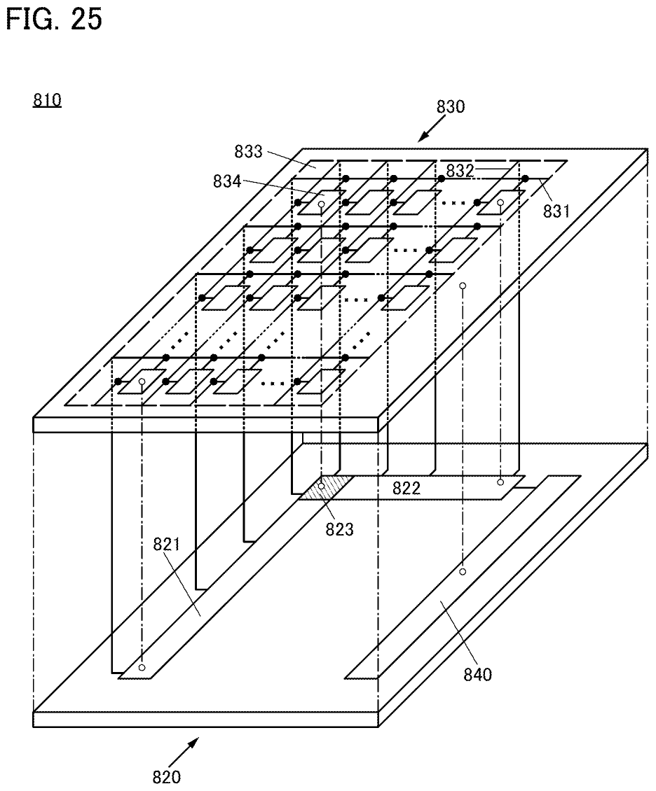

FIG. 25 is a block diagram illustrating a structure example of a display device which can be applied to an electronic device of one embodiment of the present invention. A display device 810 illustrated in FIG. 25 includes a layer 820 and a layer 830 stacked over the layer 820. The layer 820 includes a gate driver circuit 821, a source driver circuit 822, and a circuit 840. The layer 830 includes pixels 834, and the pixels 834 are arranged in a matrix to form a pixel array 833. An interlayer insulator can be provided between the layer 820 and the layer 830. Note that the layer 820 may be stacked over the layer 830.

The circuit 840 is electrically connected to the source driver circuit 822. Note that the circuit 840 may be electrically connected to another circuit or the like.

The pixels 834 in the same row are electrically connected to the gate driver circuit 821 through a wiring 831, and the pixels 834 in the same column are electrically connected to the source driver circuit 822 through a wiring 832. The wiring 831 functions as a scan line and the wiring 832 functions as a data line.

Although FIG. 25 illustrates the structure in which the pixels 834 in one row are electrically connected through one wiring 831 and the pixels 834 in one column are electrically connected through one wiring 832, one embodiment of the present invention is not limited to this structure. For example, the pixels 834 in one row may be electrically connected through two or more wirings 831, or the pixels 834 in one column may be electrically connected through two or more wirings 832. That is, for example, one pixel 834 may be electrically connected to two or more scan lines or two or more data lines. Alternatively, for example, one wiring 831 may be electrically connected to the pixels 834 in two or more rows, or one wiring 832 may be connected to the pixels 834 in two or more columns. That is, for example, one wiring 831 may be shared by the pixels 834 in two or more rows, or one wiring 832 may be shared by the pixels 834 in two or more columns.

The gate driver circuit 821 has a function of generating a signal for controlling the operation of the pixel 834 and supplying the signal to the pixel 834 through the wiring 831. The source driver circuit 822 has a function of generating an image signal and supplying the signal to the pixel 834 through the wiring 832. The circuit 840 has a function of receiving image data that serves as a base for an image signal generated by the source driver circuit 822 and supplying the received image data to the source driver circuit 822, for example. The circuit 840 also has a function of a control circuit that generates a start pulse signal, a clock signal, and the like. In addition, the circuit 840 can have a function that the gate driver circuit 821 and the source driver circuit 822 do not have.

The pixel array 833 has a function of displaying an image corresponding to image signals supplied to the pixels 834 from the source driver circuit 822. Specifically, light with luminance corresponding to the image signals is emitted from the pixels 834, whereby an image is displayed on the pixel array 833.

In FIG. 25, the positional relation between the layer 820 and the layer 830 is represented by dashed-dotted lines and blank circles; the blank circle of the layer 820 and the blank circle of the layer 830 that are connected by the dashed-dotted line overlap with each other. Note that the same representation is used in other diagrams.

In the display device 810, the gate driver circuit 821 and the source driver circuit 822, which are provided in the layer 820, each include a region overlapping with the pixel array 833. For example, the gate driver circuit 821 and the source driver circuit 822 each include a region overlapping with some of the pixels 834. Stacking the gate driver circuit 821 and the source driver circuit 822 with the pixel array 833 to have an overlap region allows the display device 810 to have a narrower bezel and a smaller size.

The gate driver circuit 821 and the source driver circuit 822 have an overlap region where they are not strictly separated from each other. The region is referred to as a region 823. With the region 823, the area occupied by the gate driver circuit 821 and the source driver circuit 822 can be reduced. Accordingly, even when the area of the pixel array 833 is small, the gate driver circuit 821 and the source driver circuit 822 can be provided without extending beyond the pixel array 833. Alternatively, the area of the region where the gate driver circuit 821 and the source driver circuit 822 do not overlap with the pixel array 833 can be reduced. In the above manner, the bezel and size can be further reduced, compared to the structure without the region 823.

The circuit 840 can be provided not to overlap with the pixel array 833. Note that the circuit 840 may be provided to have a region overlapping with the pixel array 833.

Although FIG. 25 illustrates a structure example in which one gate driver circuit 821 and one source driver circuit 822 are provided in the layer 820 and one pixel array 833 is provided in the layer 830, a plurality of pixel arrays 833 may be provided in the layer 830. That is, the pixel array provided in the layer 830 may be divided.

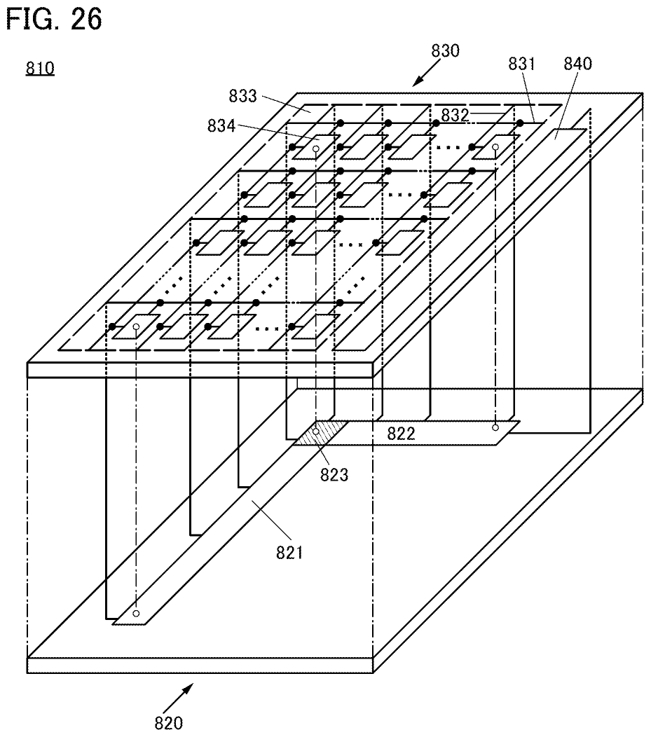

Although FIG. 25 illustrates the structure example in which the circuit 840 is provided in the layer 820, the circuit 840 is not necessarily provided in the layer 820. FIG. 26 illustrates a variation example of the structure in FIG. 25 and shows a structure example of the display device 810 in which the circuit 840 is provided in the layer 830. Note that the components of the circuit 840 may be dispersed and provided in both the layer 820 and the layer 830.

### Structure Example of Pixel 834

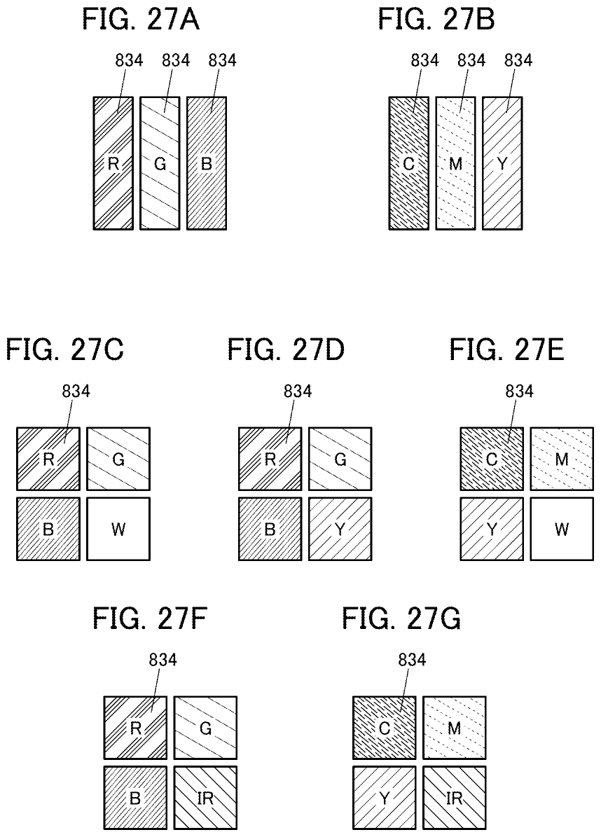

FIG. 27A to FIG. 27E are diagrams for describing colors exhibited by the pixels 834 provided in the display device 810. As illustrated in FIG. 27A, the display device in an electronic device of one embodiment of the present invention can include the pixel 834 having a function of emitting red light (R), the pixel 834 having a function of emitting green light (G), and the pixel 834 having a function of emitting blue light (B). Alternatively, as illustrated in FIG. 27B, the display device 810 may include the pixel 834 having a function of emitting cyan light (C), the pixel 834 having a function of emitting magenta light (M), and the pixel 834 having a function of emitting yellow light (Y).

Alternatively, as illustrated in FIG. 27C, the display device 810 may include the pixel 834 having a function of emitting red light (R), the pixel 834 having a function of emitting green light (G), the pixel 834 having a function of emitting blue light (B), and the pixel 834 having a function of emitting white light (W). Alternatively, as illustrated in FIG. 27D, the display device 810 may include the pixel 834 having a function of emitting red light (R), the pixel 834 having a function of emitting green light (G), the pixel 834 having a function of emitting blue light (B), and the pixel 834 having a function of emitting yellow light (Y). Alternatively, as illustrated in FIG. 27E, the display device 810 may include the pixel 834 having a function of emitting cyan light (C), the pixel 834 having a function of emitting magenta light (M), the pixel 834 having a function of emitting yellow light (Y), and the pixel 834 having a function of emitting white light (W).

Providing the pixel 834 having a function of emitting white light (W) in the display device 810 as illustrated in FIG. 27C and FIG. 27E can increase the luminance of a displayed image. Furthermore, increasing the number of colors emitted from the pixels 834 as illustrated in FIG. 27D and the like can increase the reproducibility of intermediate colors and improve the display quality.

As illustrated in FIG. 27F, the display device 810 may include the pixel 834 having a function of emitting infrared light (IR) in addition to the pixel 834 having a function of emitting red light (R), the pixel 834 having a function of emitting green light (G), and the pixel 834 having a function of emitting blue light (B). Alternatively, as illustrated in FIG. 27G, the display device 810 may include the pixel 834 having a function of emitting infrared light (IR) in addition to the pixel 834 having a function of emitting cyan light (C), the pixel 834 having a function of emitting magenta light (M), and the pixel 834 having a function of emitting yellow light (Y). Alternatively, the display device 810 may include the pixel 834 having a function of emitting white light (W) in addition to the pixels 834 illustrated in FIG. 27F or FIG. 27G.

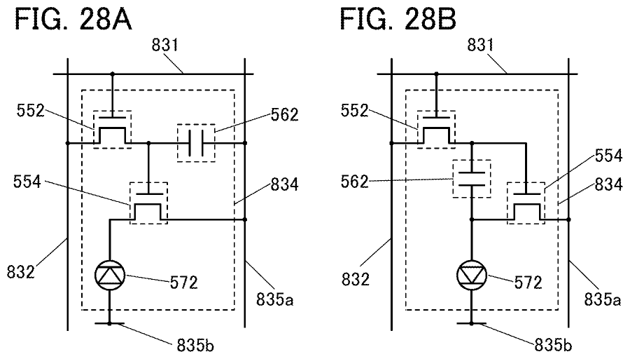

FIG. 28A and FIG. 28B are circuit diagrams each illustrating a configuration example of the pixel 834. The pixel 834 having the configuration illustrated in FIG. 28A includes a transistor 552, a transistor 554, a capacitor 562, and a light-emitting device 572. As the light-emitting device 572, an EL device utilizing electroluminescence can be used, for example. The EL device includes a layer containing a light-emitting compound (hereinafter also referred to as EL layer) between a pair of electrodes. When a potential difference that is greater than the threshold voltage of the EL device is generated between the pair of electrodes, holes are injected to the EL layer from the anode side and electrons are injected to the EL layer from the cathode side. The injected electrons and holes are recombined in the EL layer and a light-emitting substance contained in the EL layer emits light.

EL devices are classified according to whether a light-emitting material is an organic compound or an inorganic compound. In general, the former is referred to as an organic EL device, and the latter is referred to as an inorganic EL device.

In an organic EL device, by voltage application, electrons are injected from one electrode to the EL layer and holes are injected from the other electrode to the EL layer. Then, the carriers (electrons and holes) are recombined, and thus, a light-emitting organic compound is excited. The light-emitting organic compound returns to a ground state from the excited state, thereby emitting light. Owing to such a mechanism, this light-emitting device is referred to as a current-excitation light-emitting device.

In addition to the light-emitting compound, the EL layer may further include any of a substance with a high hole-injection property, a substance with a high hole-transport property, a hole-blocking material, a substance with a high electron-transport property, a substance with a high electron-injection property, a substance with a bipolar property (a substance with a high electron- and hole-transport properties), and/or the like.

The EL layer can be formed by an evaporation method (including a vacuum evaporation method), a transfer method, a printing method, an inkjet method, a coating method, or the like.

The inorganic EL devices are classified according to their device structures into a dispersion-type inorganic EL device and a thin-film inorganic EL device. A dispersion-type inorganic EL device includes a light-emitting layer where particles of a light-emitting material are dispersed in a binder, and its light emission mechanism is donor-acceptor recombination type light emission that utilizes a donor level and an acceptor level. A thin-film inorganic EL device has a structure where a light-emitting layer is positioned between dielectric layers, which are further positioned between electrodes, and its light emission mechanism is localization type light emission that utilizes inner-shell electron transition of metal ions.

In order to extract light emitted from the light-emitting device, at least one of the pair of electrodes is preferably transparent. The light-emitting device that is formed over a substrate together with a transistor can have any of a top emission structure in which emitted light is extracted through the surface opposite to the substrate; a bottom emission structure in which emitted light is extracted through the surface on the substrate side; and a dual emission structure in which emitted light is extracted through both sides.

Note that an element similar to the light-emitting device 572 can be used as light-emitting devices other than the light-emitting device 572.

One of a source and a drain of the transistor 552 is electrically connected to the wiring 832. The other of the source and the drain of the transistor 552 is electrically connected to one electrode of the capacitor 562 and a gate of the transistor 554. The other electrode of the capacitor 562 is electrically connected to a wiring 835a. A gate of the transistor 552 is electrically connected to the wiring 831. One of a source and a drain of the transistor 554 is electrically connected to the wiring 835a. The other of the source and the drain of the transistor 554 is electrically connected to one electrode of the light-emitting device 572. The other electrode of the light-emitting device 572 is electrically connected to a wiring 835b. The potential VSS is supplied to the wiring 835a, and the potential VDD is supplied to the wiring 835b. The wiring 835a and the wiring 835b function as power supply lines.

In the pixel 834 having the configuration illustrated in FIG. 28A, a current flowing through the light-emitting device 572 is controlled in accordance with a potential supplied to the gate of the transistor 554, whereby the luminance of light emitted from the light-emitting device 572 is controlled.

FIG. 28B illustrates a configuration different from that of the pixel 834 in FIG. 28A. In the pixel 834 having the configuration illustrated in FIG. 28B, one of the source and the drain of the transistor 552 is electrically connected to the wiring 832. The other of the source and the drain of the transistor 552 is electrically connected to one electrode of the capacitor 562 and the gate of the transistor 554. The gate of the transistor 552 is electrically connected to the wiring 831. One of the source and the drain of the transistor 554 is electrically connected to the wiring 835a. The other of the source and the drain of the transistor 554 is electrically connected to the other electrode of the capacitor 562 and one electrode of the light-emitting device 572. The other electrode of the light-emitting device 572 is electrically connected to the wiring 835b. The potential VDD is supplied to the wiring 835a, and the potential VSS is supplied to the wiring 835b.

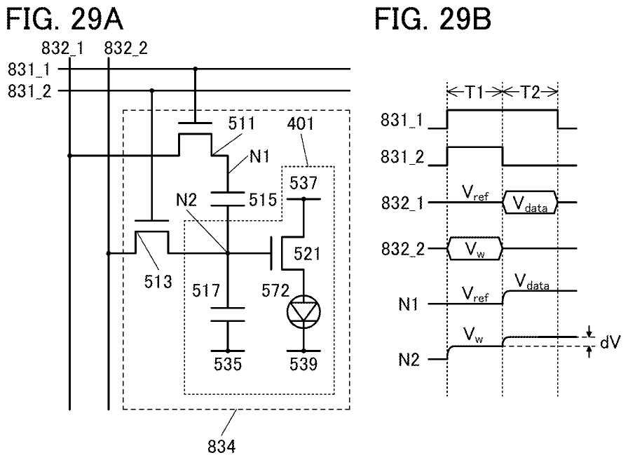

FIG. 29A illustrates a configuration example of the pixel 834 that is different from the pixels 834 having the configurations in FIG. 28A and FIG. 28B in including a memory. The pixel 834 having the configuration in FIG. 29A includes a transistor 511, a transistor 513, a transistor 521, a capacitor 515, a capacitor 517, and the light-emitting device 572. To the pixel 834, a wiring 831_1 and a wiring 831_2 are electrically connected as the wiring 831 functioning as a scan line, and a wiring 832_1 and a wiring 832_2 are electrically connected as the wiring 832 functioning as a data line.

One of a source and a drain of the transistor 511 is electrically connected to the wiring 832_1. The other of the source and the drain of the transistor 511 is electrically connected to one electrode of the capacitor 515. A gate of the transistor 511 is electrically connected to the wiring 831_1. One of a source and a drain of the transistor 513 is electrically connected to the wiring 832_2. The other of the source and the drain of the transistor 513 is electrically connected to the other electrode of the capacitor 515. A gate of the transistor 513 is electrically connected to the wiring 831_2. The other electrode of the capacitor 515 is electrically connected to one electrode of the capacitor 517. The one electrode of the capacitor 517 is electrically connected to a gate of the transistor 521. One of a source and a drain of the transistor 521 is electrically connected to one electrode of the light-emitting device 572. The other electrode of the capacitor 517 is electrically connected to a wiring 535. The other of the source and the drain of the transistor 521 is electrically connected to a wiring 537. The other electrode of the light-emitting device 572 is electrically connected to a wiring 539.

In this specification and the like, a voltage supplied to a light-emitting device indicates a difference between a potential supplied to one electrode of the light-emitting device and a potential supplied to the other electrode of the light-emitting device.

A node where the other of the source and the drain of the transistor 511 and the one electrode of the capacitor 515 are electrically connected to each other is referred to as a node N1. A node where the other of the source and the drain of the transistor 513, the one electrode of the capacitor 517, and the gate of the transistor 521 are electrically connected to each other is referred to as a node N2. In FIG. 29A, a circuit composed of the capacitor 517, the transistor 521, and the light-emitting device 572 is referred to as a circuit 401.

The wiring 535 can be shared by all pixels 834 provided in the display device 810, for example. In that case, a potential supplied to the wiring 535 is a common potential. Constant potentials can be supplied to the wiring 537 and the wiring 539. For example, a high potential can be supplied to the wiring 537, and a low potential can be supplied to the wiring 539. The wirings 537 and 539 function as power supply lines.

The transistor 521 has a function of controlling a current to be supplied to the light-emitting device 572. The capacitor 517 functions as a storage capacitor. The capacitor 517 may be omitted.

Note that FIG. 29A illustrates a configuration in which the anode of the light-emitting device 572 is electrically connected to the transistor 521; alternatively, the transistor 521 may be electrically connected to the cathode. In that case, the value of the potential of the wiring 537 and the value of the potential of the wiring 539 can be changed as appropriate.

In the pixel 834, turning off the transistor 511 enables retention of the potential of the node N1. Turning off the transistor 513 enables retention of the potential of the node N2. Furthermore, by turning off the transistor 513 and then writing a predetermined potential to the node N1 through the transistor 511, the potential of the node N2 can be changed in accordance with a change in the potential of the node N1 by capacitive coupling through the capacitor 515.

A transistor containing a metal oxide in a channel formation region (hereinafter also referred to as OS transistor) can be used as the transistor 511 and the transistor 513. A metal oxide can have a band gap of 2 eV or more, or 2.5 eV or more. Thus, an OS transistor exhibits an extremely low leakage current (off-state current) in an off state. Accordingly, the use of OS transistors as the transistor 511 and the transistor 513 enables the potentials of the node N1 and the node N2 to be held for a long time.

The metal oxide can be, for example, an In-M-Zn oxide (the element M is one or more of aluminum, gallium, yttrium, tin, copper, vanadium, beryllium, boron, titanium, iron, nickel, germanium, zirconium, molybdenum, lanthanum, cerium, neodymium, hafnium, tantalum, tungsten, magnesium, and the like). In particular, aluminum, gallium, yttrium, or tin is preferably used for the element M. Alternatively, indium oxide, zinc oxide, an In—Ga oxide, an In—Zn oxide, a Ga—Zn oxide, or gallium oxide may be used as the metal oxide.

### Example of Operation Method for Pixel 834

Next, an example of an operation method for the pixel 834 having the configuration in FIG. 29A will be described with reference to FIG. 29B. FIG. 29B is a timing chart of the operation of the pixel 834 having the configuration in FIG. 29A. Note that for simplification of the description, the influence of various kinds of resistance such as wiring resistance; parasitic capacitance of a transistor, a wiring, or the like; and the threshold voltage of a transistor, for example, is not taken into consideration here.

In the operation shown in FIG. 29B, one frame period is divided into a period T1 and a period T2. The period T1 is a period in which a potential is written to the node N2, and the period T2 is a period in which a potential is written to the node N1.

In the period T1, a potential for turning on the transistor is supplied to both the wiring 831_1 and the wiring 831_2. In addition, a potential V_{ref} that is a fixed potential is supplied to the wiring 832_1, and a potential V_{w} is supplied to the wiring 832_2.

The potential \T_{ref} is supplied from the wiring 832_1 to the node N1 through the transistor 511. The potential V_{w} is supplied from the wiring 832_2 to the node N2 through the transistor 513. Thus, a potential difference V_{w}−V_{ref} is retained in the capacitor 515.

Then, in the period T2, a potential for turning on the transistor 511 is supplied to the wiring 831_1, and a potential for turning off the transistor 513 is supplied to the wiring 831_2. A potential V_{data} is supplied to the wiring 832_1, and a predetermined constant potential is supplied to the wiring 832_2. Note that the potential of the wiring 832_2 may be floating.

The potential V_{data} is supplied to the node N1 through the transistor 511. At this time, owing to capacitive coupling through the capacitor 515, the potential of the node N2 is changed by a potential dV in accordance with the potential V_{data}. That is, a potential that is the sum of the potential V_{w} and the potential dV is input to the circuit 401. Note that although the potential dV is shown as having a positive value in FIG. 29B, the potential dV may have a negative value. That is, the potential V_{data} may be lower than the potential V_{ref}.

Here, the potential dV is roughly determined by the capacitance of the capacitor 515 and the capacitance of the circuit 401. When the capacitance of the capacitor 515 is sufficiently larger than the capacitance of the circuit 401, the potential dV becomes close to a potential difference V_{data}−V_{ref}.

As described above, the pixel 834 can generate the potential supplied to the node N2 in combination with two kinds of data signals; thus, an image displayed on the pixel array 833 can be corrected inside the pixel 834. Here, one of the two kinds of data signals can be the aforementioned image signal, and the other can be a correction signal, for example. For example, when the potential V_{w} corresponding to a correction signal is supplied to the node N2 in the period T1 and then the potential V_{data} corresponding to an image signal is supplied to the node N1 in the period T2, an image based on the image signal corrected by the correction signal can be displayed on the pixel array 833. Note that not only image signals but also correction signals and the like can be generated by the source driver circuit 822 included in the display device 810.

In the pixel 834 having the configuration in FIG. 29A, the potential of the node N2 can be set higher than the maximum potential that can be supplied to the wiring 832_1 and the wiring 832_2. Thus, a high voltage can be supplied to the light-emitting device 572. Specifically, the potential of the wiring 537 can be set higher, for example. Accordingly, when the light-emitting device 572 is an organic EL device, the light-emitting device can employ a tandem structure described later. This increases the current efficiency and external quantum efficiency of the light-emitting device 572. Thus, a high-luminance image can be displayed on the display device 810. Moreover, power consumption of the display device 810 can be reduced.

Note that the pixel configuration is not limited to that illustrated in FIG. 29A, and a transistor, a capacitor, or the like may be added. For example, when one transistor and one capacitor are added to the configuration in FIG. 29A, three nodes capable of holding a potential can be provided. That is, the pixel 834 can have another node capable of holding a potential, in addition to the node N1 and the node N2. Thus, the potential of the node N2 can be further increased. Consequently, a larger amount of current can flow through the light-emitting device 572.

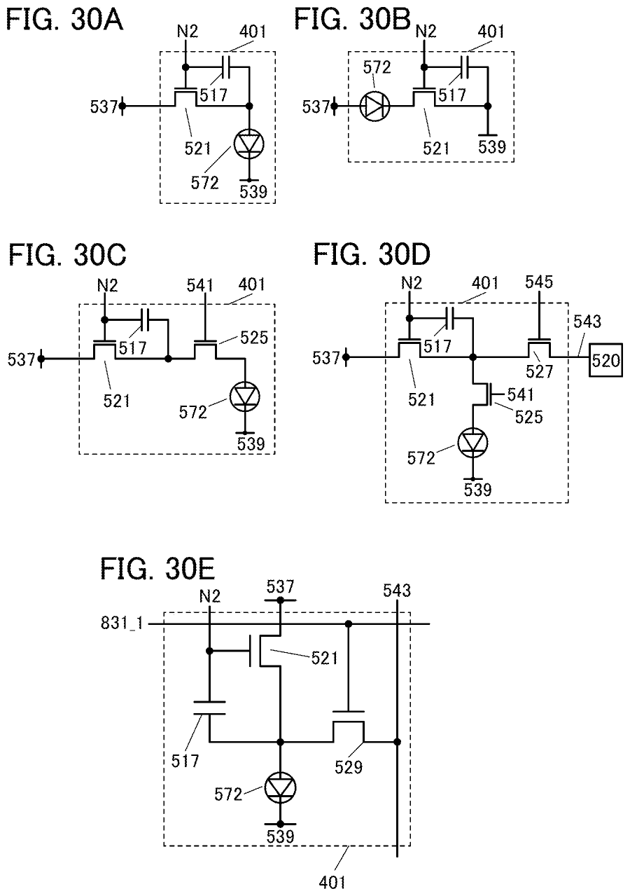

FIG. 30A to FIG. 30E illustrate configuration examples of the circuit 401 different from that in FIG. 30A. Like the circuit 401 with the configuration illustrated in FIG. 29A, the circuit 401 with the configuration illustrated in FIG. 30A includes the capacitor 517, the transistor 521, and the light-emitting device 572.

In the circuit 401 with the configuration illustrated in FIG. 30A, the gate of the transistor 521 and one electrode of the capacitor 517 are electrically connected to the node N2. One of the source and the drain of the transistor 521 is electrically connected to the wiring 537. The other of the source and the drain of the transistor 521 is electrically connected to the other electrode of the capacitor 517. The other electrode of the capacitor 517 is electrically connected to one electrode of the light-emitting device 572. The other electrode of the light-emitting device 572 is electrically connected to the wiring 539.

Like the circuit 401 with the configuration illustrated in FIG. 29A, the circuit 401 with the configuration illustrated in FIG. 30B includes the capacitor 517, the transistor 521, and the light-emitting device 572.

In the circuit 401 with the configuration illustrated in FIG. 30B, the gate of the transistor 521 and one electrode of the capacitor 517 are electrically connected to the node N2. One electrode of the light-emitting device 572 is electrically connected to the wiring 537. The other electrode of the light-emitting device 572 is electrically connected to one of the source and the drain of the transistor 521. The other of the source and the drain of the transistor 521 is electrically connected to the other electrode of the capacitor 517. The other electrode of the capacitor 517 is electrically connected to the wiring 539.

FIG. 30C illustrates a configuration example of the circuit 401 in which a transistor 525 is added to the circuit 401 in FIG. 30A. One of a source and a drain of the transistor 525 is electrically connected to the other of the source and the drain of the transistor 521 and the other electrode of the capacitor 517. The other of the source and the drain of the transistor 525 is electrically connected to one electrode of the light-emitting device 572. A gate of the transistor 525 is electrically connected to a wiring 541. The wiring 541 has a function of a scan line for controlling the conduction of the transistor 525.

In the pixel 834 including the circuit 401 with the configuration illustrated in FIG. 30C, even when the potential of the node N2 becomes higher than or equal to the threshold voltage of the transistor 521, a current does not flow through the light-emitting device 572 unless the transistor 525 is turned on. Thus, a malfunction of the display device 810 can be inhibited.

FIG. 30D illustrates a configuration example of the circuit 401 in which a transistor 527 is added to the circuit 401 in FIG. 30C. One of a source and a drain of the transistor 527 is electrically connected to the other of the source and the drain of the transistor 521. The other of the source and the drain of the transistor 527 is electrically connected to a wiring 543. A gate of the transistor 527 is electrically connected to a wiring 545. The wiring 545 has a function of a scan line for controlling the conduction of the transistor 527.

The wiring 543 can be electrically connected to a supply source of a certain potential such as a reference potential. That is, the wiring 543 has a function of a power supply line. Supplying a certain potential from the wiring 543 to the other of the source and the drain of the transistor 521 enables stable writing of an image signal to the pixel 834.

The wiring 543 can be electrically connected to a circuit 520. The circuit 520 can have at least one of a function of a supply source of the certain potential, a function of obtaining electrical characteristics of the transistor 521, and a function of generating a correction signal.

The circuit 401 having the configuration illustrated in FIG. 30E includes the capacitor 517, the transistor 521, a transistor 529, and the light-emitting device 572.

In the circuit 401 with the configuration illustrated in FIG. 30E, the gate of the transistor 521 and one electrode of the capacitor 517 are electrically connected to the node N2. One of the source and the drain of the transistor 521 is electrically connected to the wiring 537. One of a source and a drain of the transistor 529 is electrically connected to the wiring 543.

The other electrode of the capacitor 517 is electrically connected to the other of the source and the drain of the transistor 521. The other of the source and the drain of the transistor 521 is electrically connected to the other of the source and the drain of the transistor 529. The other of the source and the drain of the transistor 529 is electrically connected to one electrode of the light-emitting device 572.

A gate of the transistor 529 is electrically connected to the wiring 831_1. The other electrode of the light-emitting device 572 is electrically connected to the wiring 539.

### Structure Example 2 of Display Device

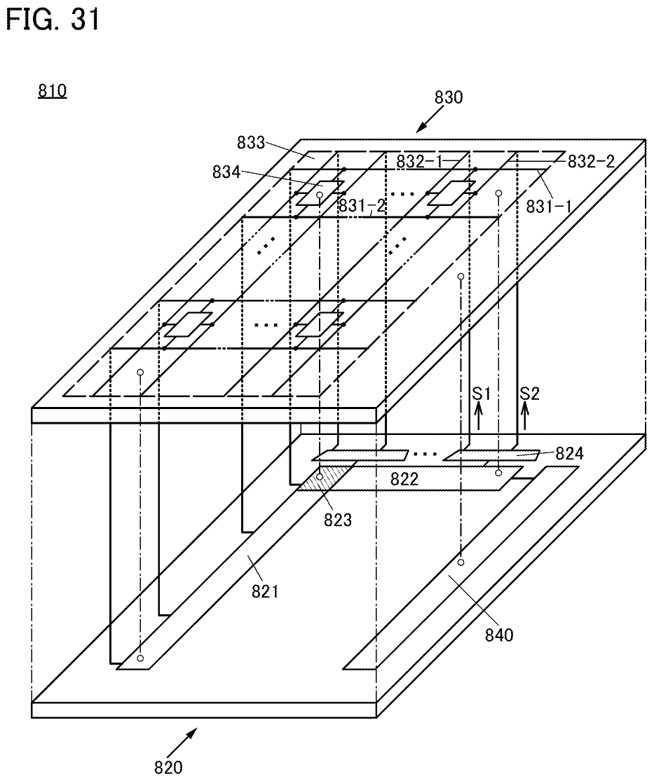

FIG. 31 is a block diagram illustrating a structure example of the display device 810 in which the pixels 834 each have the configuration illustrated in FIG. 29A. In the display device 810 having the structure illustrated in FIG. 31, a demultiplexer circuit 824 is provided in addition to the components of the display device 810 illustrated in FIG. 25. The demultiplexer circuit 824 can be provided in the layer 820 as illustrated in FIG. 31, for example. Note that the number of demultiplexer circuits 824 can be equal to the number of columns of the pixels 834 arranged in the pixel array 833, for example.

The gate driver circuit 821 is electrically connected to the pixels 834 through wirings 831-1. The gate driver circuit 821 is electrically connected to the pixels 834 through wirings 831-2. The wirings 831-1 and the wirings 831-2 function as scan lines.

The source driver circuit 822 is electrically connected to an input terminal of the demultiplexer circuit 824. A first output terminal of the demultiplexer circuit 824 is electrically connected to the pixel 834 through a wiring 832-1. A second output terminal of the demultiplexer circuit 824 is electrically connected to the pixel 834 through a wiring 832-2. The wiring 832-1 and the wiring 832-2 function as data lines.

Note that the source driver circuit 822 and the demultiplexer circuits 824 may be collectively referred to as a source driver circuit. In other words, the demultiplexer circuits 824 may be included in the source driver circuit 822.

In the display device 810 having the structure in FIG. 31, the source driver circuit 822 has a function of generating an image signal S1 and an image signal S2. The demultiplexer circuit 824 has a function of supplying the image signal S1 to the pixel 834 through the wiring 832-1, and a function of supplying the image signal S2 to the pixel 834 through the wiring 832-2.

Here, when the display device 810 having the structure in FIG. 31 operates with the method illustrated in FIG. 29B, the potential V_{data} can be a potential corresponding to the image signal S1 and the potential V_{w} can be a potential corresponding to the image signal S2.

When the potential V_{w} is supplied to the node N2 and then the potential V_{data} is supplied to the node N1 as shown in FIG. 29B, the potential of the node N2 becomes “V_{w}+dV”. Here, the potential dV corresponds to the potential V_{data} as described above. As a result, the image signal S1 can be added to the image signal S2. That is, the image signal S1 can be superimposed on the image signal S2.

The level of the potential V_{data} corresponding to the image signal S1 and the level of the potential V_{w} corresponding to the image signal S2 are limited by the withstand voltage of the source driver circuit 822, for example. In view of this, superimposing the image signal S1 and the image signal S2 enables an image corresponding to an image signal having a potential higher than a potential that the source driver circuit 822 can output, to be displayed on the pixel array 833. Thus, a large amount of current can flow through the light-emitting device 572; hence, the pixel array 833 can display a high-luminance image. Moreover, the dynamic range, which is the range of luminance of images that the pixel array 833 can display, can be enlarged.

An image corresponding to the image signal S1 and an image corresponding to the image signal S2 may be the same or different from each other. When an image corresponding to the image signal S1 and an image corresponding to the image signal S2 are the same, the pixel array 833 can display an image with higher luminance than the luminance of the image corresponding to either the image signal S1 or the image signal S2.

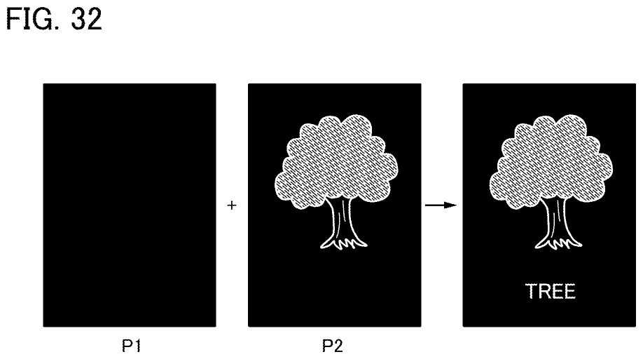

FIG. 32 shows the case where an image P1 corresponding to the image signal S1 includes only letters, and an image P2 corresponding to the image signal S2 includes a picture and letters. In this case, when the image P1 and the image P2 are superimposed on each other, the luminance of the letters can be increased, whereby the letters can be emphasized, for example. As illustrated in FIG. 29B, the potential of the node N2 is changed in accordance with the potential Vaasa after the potential V_{w} is written to the node N2; hence, to rewrite the potential V_{w} corresponding to the image signal S2, the potential V_{data} of the image signal S1 needs to be written again. Meanwhile, to rewrite the potential V_{data}, the potential V_{w} does not need to be rewritten as long as the charge written to the node N2 at the period T1 shown in FIG. 29B is retained without being leaked through the transistor 513 or the like. Therefore, in the case illustrated in FIG. 32, the luminance of the letters can be controlled by adjusting the level of the potential V_{data}.

Here, to rewrite the potential V_{w} corresponding to the image signal S2, the potential V_{data} corresponding to the image signal S1 needs to be written again as described above. On the other hand, to rewrite the potential V_{data}, the potential V_{w} does not need to be rewritten. Therefore, the image P2 is preferably an image that needs to be rewritten less frequently than the image P1. FIG. 32 illustrates the example in which the image P1 includes only letters and the image P2 includes a picture and letters; however, one embodiment of the present invention is not limited to the example.

### Example of Cross-Sectional Structure of Display Device

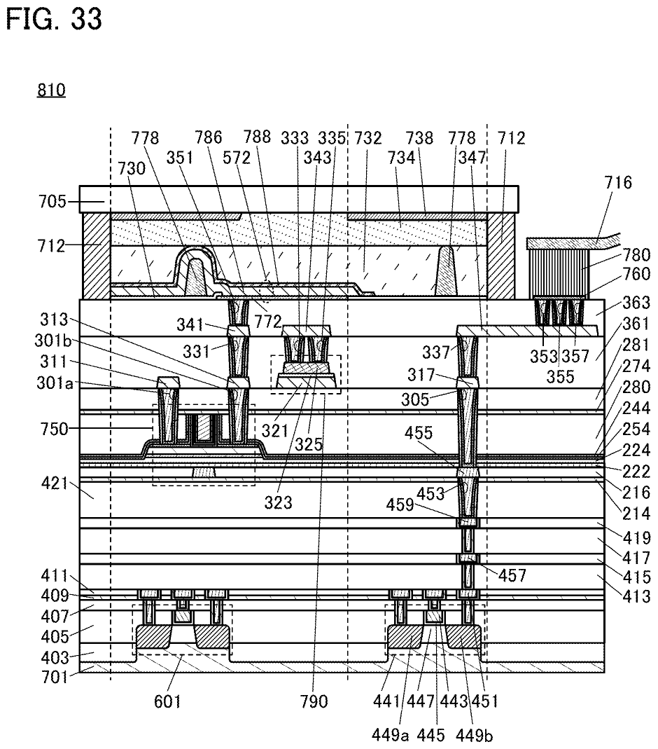

FIG. 33 is a cross-sectional view illustrating a structure example of the display device 810. The display device 810 includes a substrate 701 and a substrate 705. The substrate 701 and the substrate 705 are attached to each other with a sealant 712.

As the substrate 701, a single crystal semiconductor substrate such as a single crystal silicon substrate can be used. Note that a semiconductor substrate other than a single crystal semiconductor substrate may be used as the substrate 701.

A transistor 441 and a transistor 601 are provided on the substrate 701. The transistor 441 can be a transistor provided in the circuit 840. The transistor 601 can be a transistor provided in the gate driver circuit 821 or a transistor provided in the source driver circuit 822. That is, the transistor 441 and the transistor 601 can be provided in the layer 820 illustrated in the drawings like FIG. 25.

The transistor 441 is formed of a conductor 443 functioning as a gate electrode, an insulator 445 functioning as a gate insulator, and part of the substrate 701 and includes a semiconductor region 447 including a channel formation region, a low-resistance region 449a functioning as one of a source region and a drain region, and a low-resistance region 449b functioning as the other of the source region and the drain region. The transistor 441 can be a p-channel transistor or an n-channel transistor.

The transistor 441 is electrically isolated from other transistors by an element isolation layer 403. FIG. 33 illustrates the case where the transistor 441 and the transistor 601 are electrically isolated from each other by the element isolation layer 403. The element isolation layer 403 can be formed by a local oxidation of silicon (LOCOS) method, a shallow trench isolation (STI) method, or the like.

Here, in the transistor 441 illustrated in FIG. 33, the semiconductor region 447 has a projecting shape. Moreover, the conductor 443 is provided to cover a side surface and a top surface of the semiconductor region 447 with the insulator 445 therebetween. Note that FIG. 33 does not illustrate the state where the conductor 443 covers the side surface of the semiconductor region 447. A material for adjusting the work function can be used for the conductor 443.

A transistor having a projecting semiconductor region, like the transistor 441, can be referred to as a fin-type transistor because a projecting portion of a semiconductor substrate is used. An insulator functioning as a mask for forming a projecting portion may be provided in contact with the top surface of the projecting portion. Although FIG. 33 illustrates the structure in which the projecting portion is formed by processing part of the substrate 701, a semiconductor having a projecting shape may be formed by processing an SOI substrate.

Note that the structure of the transistor 441 illustrated in FIG. 33 is one example; the structure of the transistor 441 is not limited to the example and can be changed as appropriate in accordance with the circuit configuration, an operation method for the circuit, or the like. For example, the transistor 441 may be a planar transistor.

The transistor 601 can have the same structure as the transistor 441.

An insulator 405, an insulator 407, an insulator 409, and an insulator 411 are provided over the substrate 701, in addition to the element isolation layer 403, the transistor 441, and the transistor 601. A conductor 451 is embedded in the insulator 405, the insulator 407, the insulator 409, and the insulator 411. Here, the top surface of the conductor 451 and the top surface of the insulator 411 can be substantially level with each other.

An insulator 413 and an insulator 415 are provided over the conductor 451 and the insulator 411. A conductor 457 is embedded in the insulator 413 and the insulator 415.

An insulator 417 and an insulator 419 are provided over the conductor 457 and the insulator 415. A conductor 459 is embedded in the insulator 417 and the insulator 419.

An insulator 421 and an insulator 214 are provided over the conductor 459 and the insulator 419. A conductor 453 is embedded in the insulator 421 and the insulator 214. Here, the top surface of the conductor 453 and the top surface of the insulator 214 can be substantially level with each other.

An insulator 216 is provided over the conductor 453 and the insulator 214. A conductor 455 is embedded in the insulator 216. Here, the top surface of the conductor 455 and the top surface of the insulator 216 can be substantially level with each other.

An insulator 222, an insulator 224, an insulator 254, an insulator 244, an insulator 280, an insulator 274, and an insulator 281 are provided over the conductor 455 and the insulator 216. A conductor 305 is embedded in the insulator 222, the insulator 224, the insulator 254, the insulator 244, the insulator 280, the insulator 274, and the insulator 281. Here, the top surface of the conductor 305 and the top surface of the insulator 281 can be substantially level with each other.

An insulator 361 is provided over the conductor 305 and the insulator 281. A conductor 317 and a conductor 337 are embedded in the insulator 361. Here, the top surface of the conductor 337 and the top surface of the insulator 361 can be substantially level with each other.

An insulator 363 is provided over the conductor 337 and the insulator 361. A conductor 347, a conductor 353, a conductor 355, and a conductor 357 are embedded in the insulator 363. Here, the top surfaces of the conductor 353, the conductor 355, and the conductor 357 and the top surface of the insulator 363 can be substantially level with each other.

A connection electrode 760 is provided over the conductor 353, the conductor 355, the conductor 357, and the insulator 363. An anisotropic conductor 780 is provided to be electrically connected to the connection electrode 760. A flexible printed circuit (FPC) 716 is provided to be electrically connected to the anisotropic conductor 780. A variety of signals and the like are supplied to the display device 810 from the outside of the display device 810 through the FPC 716.

As illustrated in FIG. 33, the low-resistance region 449b functioning as the other of the source region and the drain region of the transistor 441 is electrically connected to the FPC 716 through the conductor 451, the conductor 457, the conductor 459, the conductor 453, the conductor 455, the conductor 305, the conductor 317, the conductor 337, the conductor 347, the conductor 353, the conductor 355, the conductor 357, the connection electrode 760, and the anisotropic conductor 780. Although FIG. 33 illustrates three conductors, the conductor 353, the conductor 355, and the conductor 357, as conductors that electrically connect the connection electrode 760 and the conductor 347, one embodiment of the present invention is not limited thereto. The number of conductors having a function of electrically connecting the connection electrode 760 and the conductor 347 may be one, two, or four or more. Providing a plurality of conductors having a function of electrically connecting the connection electrode 760 and the conductor 347 can reduce the contact resistance.

A transistor 750 is provided over the insulator 214. The transistor 750 can be a transistor provided in the pixel 834. That is, the transistor 750 can be provided in the layer 830 illustrated in the drawings like FIG. 25. An OS transistor can be used as the transistor 750. Owing to an extremely low off-state current of the OS transistor, an image signal or the like can be held for a longer time, so that the refresh operation can be less frequent. Thus, power consumption of the display device 810 can be reduced.

A conductor 301a and a conductor 301b are embedded in the insulator 254, the insulator 244, the insulator 280, the insulator 274, and the insulator 281. The conductor 301a is electrically connected to one of the source and the drain of the transistor 750, and the conductor 301b is electrically connected to the other of the source and the drain of the transistor 750. Here, the top surfaces of the conductor 301a and the conductor 301b and the top surface of the insulator 281 can be substantially level with each other.

A conductor 311, a conductor 313, a conductor 331, a capacitor 790, a conductor 333, and a conductor 335 are embedded in the insulator 361. The conductor 311 and the conductor 313 are electrically connected to the transistor 750 and serve as wirings. The conductor 333 and the conductor 335 are electrically connected to the capacitor 790. Here, the top surfaces of the conductor 331, the conductor 333, and the conductor 335 and the top surface of the insulator 361 can be substantially level with each other.

A conductor 341, a conductor 343, and a conductor 351 are embedded in the insulator 363. Here, the top surface of the conductor 351 and the top surface of the insulator 363 can be substantially level with each other.

The insulator 405, the insulator 407, the insulator 409, the insulator 411, the insulator 413, the insulator 415, the insulator 417, the insulator 419, the insulator 421, the insulator 214, the insulator 280, the insulator 274, the insulator 281, the insulator 361, and the insulator 363 function as an interlayer film and may also function as a planarization film that covers unevenness thereunder. For example, the top surface of the insulator 363 may be planarized by planarization treatment using a chemical mechanical polishing (CMP) method or the like to increase the level of planarity.

As illustrated in FIG. 33, the capacitor 790 includes a lower electrode 321 and an upper electrode 325. An insulator 323 is provided between the lower electrode 321 and the upper electrode 325. That is, the capacitor 790 has a stacked-layer structure in which the insulator 323 functioning as a dielectric is positioned between the pair of electrodes. Although FIG. 33 illustrates an example in which the capacitor 790 is provided over the insulator 281, the capacitor 790 may be provided over an insulator other than the insulator 281.

In the example illustrated in FIG. 33, the conductor 301a, the conductor 301b, and the conductor 305 are formed in the same layer. The conductor 311, the conductor 313, and the conductor 317 and the lower electrode 321 are formed in the same layer. The conductor 331, the conductor 333, the conductor 335, and the conductor 337 are formed in the same layer. The conductor 341, the conductor 343, and the conductor 347 are formed in the same layer. The conductor 351, the conductor 353, the conductor 355, and the conductor 357 are formed in the same layer. Forming a plurality of conductors in the same layer in this manner simplifies the process of manufacturing the display device 810 and thus makes the display device 810 inexpensive. Note that these conductors may be formed in different layers or may contain different types of materials.

The display device 810 illustrated in FIG. 33 includes the light-emitting device 572.

The light-emitting device 572 includes a conductor 772, an EL layer 786, and a conductor 788. The conductor 788 is provided on the substrate 705 side and functions as a common electrode. The conductor 772 is electrically connected to the other of the source and the drain of the transistor 750 through the conductor 351, the conductor 341, the conductor 331, the conductor 313, and the conductor 301b. The conductor 772 is formed over the insulator 363 and functions as a pixel electrode. The EL layer 786 contains an organic compound or an inorganic compound such as a quantum dot.

Examples of materials that can be used for an organic compound include a fluorescent material and a phosphorescent material. Examples of materials that can be used for a quantum dot include a colloidal quantum dot material, an alloyed quantum dot material, a core-shell quantum dot material, and a core quantum dot material.

In the display device 810 illustrated in FIG. 33, an insulator 730 is provided over the insulator 363. Here, the insulator 730 can cover part of the conductor 772. The light-emitting device 572 includes the light-transmitting conductor 788 and thus can be a top-emission light-emitting device. Note that the light-emitting device 572 may have a bottom-emission structure in which light is emitted towards the conductor 772 or a dual-emission structure in which light is emitted towards both the conductor 772 and the conductor 788.

The light-emitting device 572 can have a microcavity structure, which will be described later in detail. Thus, light of predetermined colors (e.g., RGB) can be extracted without a coloring layer, and the display device 810 can perform color display. The structure without a coloring layer can prevent light absorption due to the coloring layer. As a result, the display device 810 can display high-luminance images, and power consumption of the display device 810 can be reduced. Note that a structure without a coloring layer can be employed even when the EL layer 786 is formed into an island shape for each pixel or formed into a stripe shape for each pixel column, i.e., the EL layers 786 are formed separately for respective colors.

A light-blocking layer 738 is provided to include a region overlapping with the insulator 730. The light-blocking layer 738 is covered with an insulator 734. A space between the light-emitting device 572 and the insulator 734 is filled with a sealing layer 732.

A component 778 is provided between the insulator 730 and the EL layer 786. Another component 778 is provided between the insulator 730 and the insulator 734. The component 778 is a columnar spacer and has a function of controlling the distance (cell gap) between the substrate 701 and the substrate 705. Note that a spherical spacer may be used as the component 778.

The light-blocking layer 738 and the insulator 734 that is in contact with the light-blocking layer 738 are provided on the substrate 705 side. The light-blocking layer 738 has a function of blocking light emitted from adjacent regions. Alternatively, the light-blocking layer 738 has a function of preventing external light from reaching the transistor 750 or the like.

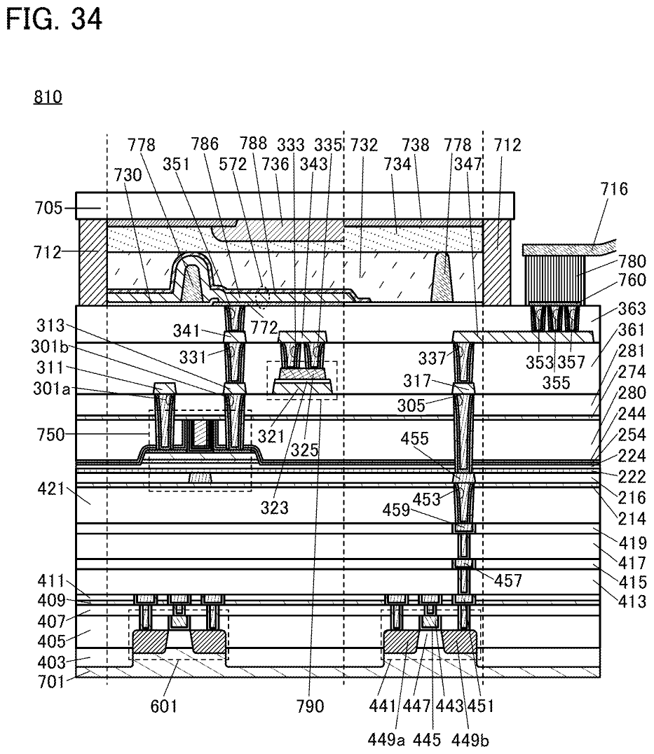

FIG. 34 illustrates a variation example of the display device 810 in FIG. 33. The display device 810 in FIG. 34 is different from the display device 810 in FIG. 33 in including a coloring layer 736. Providing the coloring layer 736 can improve the color purity of light extracted from the light-emitting device 572. Thus, the display device 810 can display high-quality images. Furthermore, all the light-emitting devices 572, for example, in the display device 810 can be light-emitting devices that emit white light; hence, it is unnecessary to separately form the EL layers 786 for respective colors and thus the display device 810 with high resolution can be obtained.

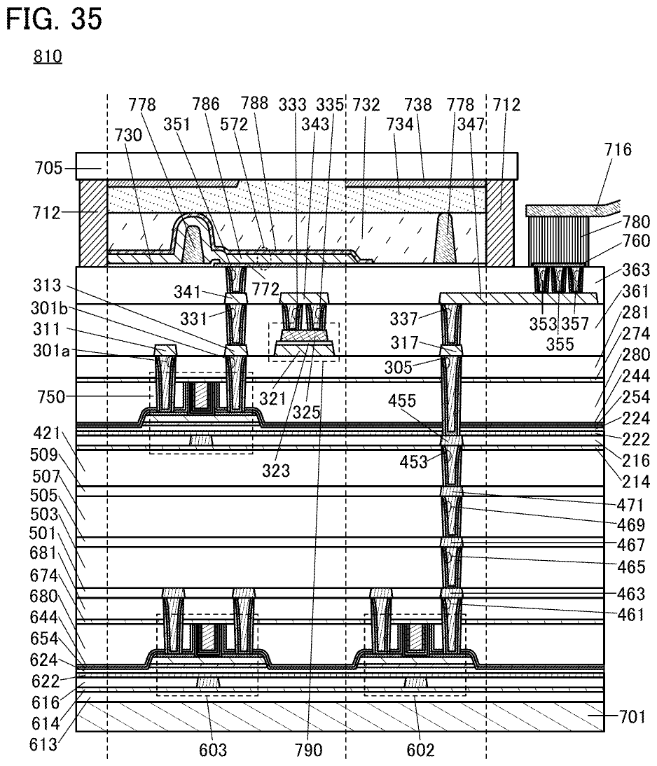

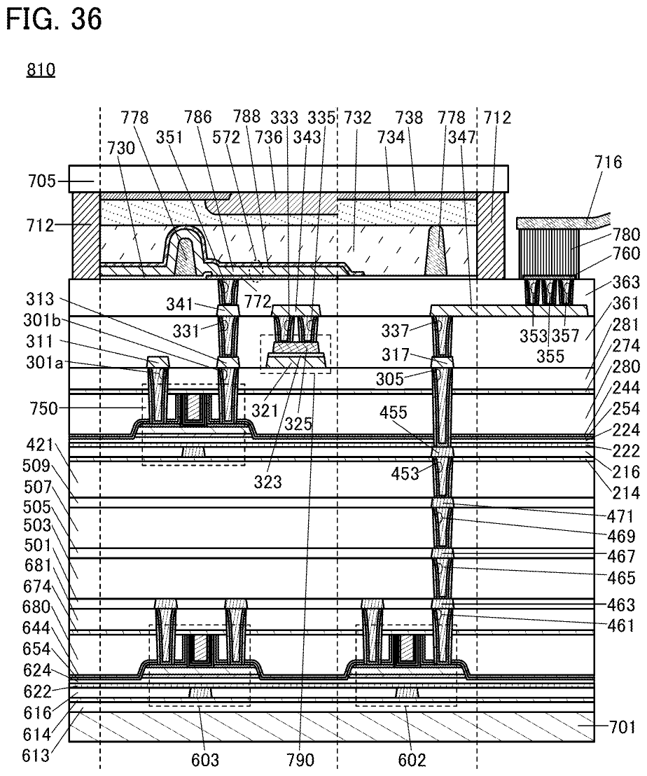

Although FIG. 33 and FIG. 34 each illustrate a structure where the transistor 441 and the transistor 601 are provided so that their channel formation regions are formed inside the substrate 701 and the OS transistor is stacked over the transistor 441 and the transistor 601, one embodiment of the present invention is not limited thereto. FIG. 35 illustrates a variation example of FIG. 33, and FIG. 36 illustrates a variation example of FIG. 34. The display devices 810 in FIG. 35 and FIG. 36 differs from the display devices 810 with the structures in FIG. 33 and FIG. 34 in that the transistor 750 is stacked over a transistor 602 and a transistor 603 that are OS transistors, instead of over the transistor 441 and the transistor 601. That is, the display devices 810 with the structures in FIG. 35 and FIG. 36 each include a stack of OS transistors.

An insulator 613 and an insulator 614 are provided over the substrate 701, and the transistor 602 and the transistor 603 are provided over the insulator 614. Note that a transistor or the like may be provided between the substrate 701 and the insulator 613. For example, a transistor having a structure similar to that of the transistor 441 and the transistor 601 illustrated in FIG. 33 and FIG. 34 may be provided between the substrate 701 and the insulator 613.

The transistor 602 can be a transistor provided in the circuit 840. The transistor 603 can be a transistor provided in the gate driver circuit 821 or a transistor provided in the source driver circuit 822. That is, the transistor 602 and the transistor 603 can be provided in the layer 820 illustrated in the drawings like FIG. 25. Note that when the circuit 840 is provided in the layer 830 as illustrated in FIG. 26, the transistor 602 can be provided in the layer 830.

The transistor 602 and the transistor 603 can have a structure similar to that of the transistor 750. Note that the transistor 602 and the transistor 603 may be OS transistors having a structure different from that of the transistor 750.

An insulator 616, an insulator 622, an insulator 624, an insulator 654, an insulator 644, an insulator 680, an insulator 674, and an insulator 681 are provided over the insulator 614, in addition to the transistor 602 and the transistor 603. A conductor 461 is embedded in the insulator 654, the insulator 644, the insulator 680, the insulator 674, and the insulator 681. Here, the top surface of the conductor 461 and the top surface of the insulator 681 can be substantially level with each other.

An insulator 501 is provided over the conductor 461 and the insulator 681. A conductor 463 is embedded in the insulator 501. Here, the top surface of the conductor 463 and the top surface of the insulator 501 can be substantially level with each other.

An insulator 503 is provided over the conductor 463 and the insulator 501. A conductor 465 is embedded in the insulator 503. The top surface of the conductor 465 and the top surface of the insulator 503 can be substantially level with each other.

An insulator 505 is provided over the conductor 465 and the insulator 503. A conductor 467 is embedded in the insulator 505.

An insulator 507 is provided over the conductor 467 and the insulator 505. A conductor 469 is embedded in the insulator 507. Here, the top surface of the conductor 469 and the top surface of the insulator 507 can be substantially level with each other.

An insulator 509 is provided over the conductor 469 and the insulator 507. A conductor 471 is embedded in the insulator 509.

The insulator 421 and the insulator 214 are provided over the conductor 471 and the insulator 509. The conductor 453 is embedded in the insulator 421 and the insulator 214. Here, the top surface of the conductor 453 and the top surface of the insulator 214 can be substantially level with each other.

As illustrated in FIG. 35 and FIG. 36, one of a source and a drain of the transistor 602 is electrically connected to the FPC 716 through the conductor 461, the conductor 463, the conductor 465, the conductor 467, the conductor 469, the conductor 471, the conductor 453, the conductor 455, the conductor 305, the conductor 317, the conductor 337, the conductor 347, the conductor 353, the conductor 355, the conductor 357, the connection electrode 760, and the anisotropic conductor 780.

The insulator 613, the insulator 614, the insulator 680, the insulator 674, the insulator 681, the insulator 501, the insulator 503, the insulator 505, the insulator 507, and the insulator 509 function as interlayer films and may also function as planarization films that cover unevenness thereunder.

When the display device 810 has the structure illustrated in FIG. 35 or FIG. 36, all the transistors in the display device 810 can be OS transistors while the bezel and size of the display device 810 are reduced. Accordingly, the transistors provided in the layer 820 and the transistors provided in the layer 830 can be manufactured using the same apparatus, for example. Consequently, the manufacturing cost of the display device 810 can be reduced, making the display device 810 inexpensive.

### Structure Example of Light-Emitting Device

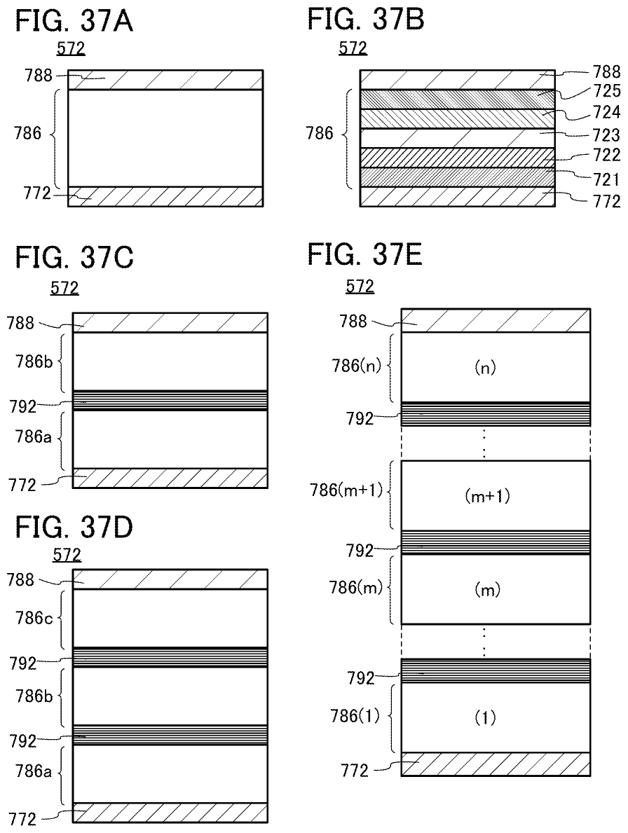

FIG. 37A to FIG. 37E illustrate structure examples of the light-emitting device 572. FIG. 37A illustrates a structure where the EL layer 786 is positioned between the conductor 772 and the conductor 788 (a single structure). As described above, the EL layer 786 contains a light-emitting material, for example, a light-emitting material of an organic compound.

FIG. 37B illustrates a stacked-layer structure of the EL layer 786. In the light-emitting device 572 with the structure illustrated in FIG. 37B, the conductor 772 functions as an anode and the conductor 788 functions as a cathode.

The EL layer 786 has a structure in which a hole-injection layer 721, a hole-transport layer 722, a light-emitting layer 723, an electron-transport layer 724, and an electron-injection layer 725 are stacked in this order over the conductor 772. Note that the order of the stacked layers is reversed when the conductor 772 functions as a cathode and the conductor 788 functions as an anode.

The light-emitting layer 723 contains a light-emitting material and a plurality of materials in appropriate combination, so that fluorescence or phosphorescence of a desired emission color can be obtained. The light-emitting layer 723 may have a stacked-layer structure having different emission colors. In that case, the light-emitting substance and other substances may be different between the stacked light-emitting layers.

For example, when the light-emitting device 572 has a micro optical resonator (microcavity) structure with the conductor 772 and the conductor 788 in FIG. 37B serving as a reflective electrode and a transflective electrode, respectively, light emitted from the light-emitting layer 723 in the EL layer 786 can be resonated between the electrodes and thus the light emitted through the conductor 788 can be intensified.

Note that when the conductor 772 of the light-emitting device 572 is a reflective electrode having a stacked-layer structure of a reflective conductive material and a light-transmitting conductive material (transparent conductive film), optical adjustment can be performed by controlling the thickness of the transparent conductive film. Specifically, when the wavelength of light from the light-emitting layer 723 is X, the interelectrode distance between the conductor 772 and the conductor 788 is preferably adjusted to around mλ/2 (m is a natural number).

To amplify desired light (wavelength: λ) obtained from the light-emitting layer 723, the optical path length from the conductor 772 to a region where desired light is obtained in the light-emitting layer (light-emitting region) and the optical path length from the conductor 788 to the region where desired light is obtained in the light-emitting layer 723 (light-emitting region) are preferably adjusted to around (2m′+1)λ/4 (m′ is a natural number). Here, the light-emitting region means a region where holes and electrons are recombined in the light-emitting layer 723.

By such optical adjustment, the spectrum of specific monochromatic light emitted from the light-emitting layer 723 can be narrowed and light emission with high color purity can be obtained.

In the above case, the optical path length between the conductor 772 and the conductor 788 is, to be exact, the total thickness between a reflective region in the conductor 772 and a reflective region in the conductor 788. However, it is difficult to precisely determine the reflection region in the conductor 772 and the conductor 788; hence, it is assumed that the above effect is adequately obtained wherever the reflective region is placed in the conductor 772 and the conductor 788. Furthermore, the optical path length between the conductor 772 and the light-emitting layer emitting desired light is, to be exact, the optical path length between the reflective region in the conductor 772 and the light-emitting region where desired light is obtained in the light-emitting layer. However, it is difficult to precisely determine the reflective region in the conductor 772 and the light-emitting region where desired light is obtained in the light-emitting layer; thus, it is assumed that the above effect is adequately obtained wherever the reflective region and the light-emitting region are placed in the conductor 772 and the light-emitting layer emitting desired light.

The light-emitting device 572 illustrated in FIG. 37B has a microcavity structure, so that light (monochromatic light) with different wavelengths can be extracted from different light-emitting devices including the same EL layer. Thus, separate formation for obtaining different emission colors (e.g., R, G, and B) is not necessary. Therefore, high resolution can be easily achieved. Note that a combination with coloring layers is also possible. Furthermore, the emission intensity of light with a specific wavelength in the front direction can be increased, whereby power consumption can be reduced.

Note that the light-emitting device 572 illustrated in FIG. 37B does not necessarily have a microcavity structure. In the case where a microcavity structure is not employed, light of predetermined colors (e.g., RGB) can be extracted when the light-emitting layer 723 has a structure for emitting white light and coloring layers are provided. When the EL layers 786 are separately formed for obtaining different emission colors, light of predetermined colors can be extracted without providing coloring layers.

At least one of the conductors 772 and 788 can be a light-transmitting electrode (e.g., a transparent electrode or a transflective electrode). In the case where the light-transmitting electrode is a transparent electrode, the transparent electrode has a visible light transmittance of higher than or equal to 40%. In the case where the light-transmitting electrode is a transflective electrode, the transflective electrode has a visible light reflectance of higher than or equal to 20% and lower than or equal to 80%, preferably higher than or equal to 40% and lower than or equal to 70%. These electrodes preferably have a resistivity of 1×10⁻² Ωcm or less.

When the conductor 772 or the conductor 788 is an electrode having reflectivity (reflective electrode), the visible light reflectance of the reflective electrode is higher than or equal to 40% and lower than or equal to 100%, preferably higher than or equal to 70% and lower than or equal to 100%. This electrode preferably has a resistivity of 1×10⁻² Ωcm or less.

The light-emitting device 572 may have a structure illustrated in FIG. 37C. FIG. 37C illustrates the light-emitting device 572 having a stacked-layer structure (tandem structure) in which two EL layers (an EL layer 786a and an EL layer 786b) are provided between the conductor 772 and the conductor 788, and a charge generation layer 792 is provided between the EL layer 786a and the EL layer 786b. When the light-emitting device 572 has the tandem structure, the current efficiency and external quantum efficiency of the light-emitting device 572 can be increased. Therefore, the display device 810 can display high-luminance images. Moreover, power consumption of the display device 810 can be reduced. Here, the EL layer 786a and the EL layer 786b can have a structure similar to that of the EL layer 786 illustrated in FIG. 37B.

The charge generation layer 792 has a function of injecting electrons into one of the EL layer 786a and the EL layer 786b and injecting holes to the other of the EL layer 786a and the EL layer 786b when a voltage is supplied between the conductor 772 and the conductor 788. Accordingly, when a voltage is supplied such that the potential of the conductor 772 becomes higher than the potential of the conductor 788, electrons are injected into the EL layer 786a from the charge generation layer 792 and holes are injected into the EL layer 786b from the charge generation layer 792.

Note that in terms of light extraction efficiency, the charge generation layer 792 preferably transmits visible light (specifically, the visible light transmittance of the charge generation layer 792 is preferably 40% or higher). The conductivity of the charge generation layer 792 may be lower than that of the conductor 772 or the conductor 788.

The light-emitting device 572 may have a structure illustrated in FIG. 37D. FIG. 37D illustrates the light-emitting device 572 having a tandem structure in which three EL layers (the EL layer 786a, the EL layer 786b, and an EL layer 786c) are provided between the conductor 772 and the conductor 788, and the charge generation layer 792 is provided between the EL layer 786a and the EL layer 786b and between the EL layer 786b and the EL layer 786c each. Here, the EL layer 786a, the EL layer 786b, and the EL layer 786c can have a structure similar to that of the EL layer 786 illustrated in FIG. 37B. When the light-emitting device 572 has the structure illustrated in FIG. 37D, the current efficiency and external quantum efficiency of the light-emitting device 572 can be further increased. As a result, the display device 810 can display higher-luminance images. Moreover, power consumption of the display device 810 can be further reduced.

The light-emitting device 572 may have a structure illustrated in FIG. 37E. FIG. 37E illustrates the light-emitting device 572 having a tandem structure in which n EL layers (EL layer 786(1) to EL layer 786(n)) are provided between the conductor 772 and the conductor 788, and the charge generation layer 792 is provided between the EL layers 786. Here, the EL layer 786(1) to the EL layer 786(n) can have a structure similar to that of the EL layer 786 illustrated in FIG. 37B. Note that FIG. 37E illustrates the EL layer 786(1), the EL layer 786(m), the EL layer 786(m+1), and the EL layer 786(n) among the EL layers 786. Here, m is an integer greater than or equal to 2 and less than n, and n is an integer greater than m. As n becomes larger, the current efficiency and external quantum efficiency of the light-emitting device 572 can be increased. As a result, the display device 810 can display high-luminance images. Moreover, power consumption of the display device 810 can be reduced.

<Materials for Light-Emitting Device>

Next, materials that can be used for the light-emitting device 572 will be described.

<<Conductor 772 and Conductor 788>>

For the conductor 772 and the conductor 788, any of the following materials can be used in an appropriate combination as long as the a function of the anode and the cathode can be fulfilled. For example, a metal, an alloy, an electrically conductive compound, a mixture of these, and the like can be appropriately used. Specifically, an In—Sn oxide (also referred to as ITO), an In—Si—Sn oxide (also referred to as ITSO), an In—Zn oxide, or an In—W—Zn oxide can be used. In addition, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use a Group 1 element or a Group 2 element in the periodic table, which is not described above (e.g., lithium (Li), cesium (Cs), calcium (Ca), or strontium (Sr)), a rare earth metal such as europium (Eu) or ytterbium (Yb), an alloy containing an appropriate combination of any of these elements, graphene, or the like.

<<Hole-Injection Layer 721 and Hole-Transport Layer 722>>

The hole-injection layer 721 injects holes to the EL layer 786 from the conductor 772, which is an anode, or the charge generation layer 792 and contains a material with a high hole-injection property. Here, the EL layer 786 includes the EL layer 786a, the EL layer 786b, the EL layer 786c, and the EL layer 786(1) to the EL layer 786(n).

Examples of the material with a high hole-injection property include transition metal oxides such as molybdenum oxide, vanadium oxide, ruthenium oxide, tungsten oxide, and manganese oxide. Alternatively, it is possible to use a phthalocyanine-based compound, an aromatic amine compound, a high molecular compound, or the like.

Alternatively, as the material with a high hole-injection property, a composite material containing a hole-transport material and an acceptor material (electron-accepting material) can be used. In that case, the acceptor material extracts electrons from the hole-transport material, so that holes are generated in the hole-injection layer 721 and the holes are injected into the light-emitting layer 723 through the hole-transport layer 722. Note that the hole-injection layer 721 may be formed to have a single-layer structure using a composite material containing a hole-transport material and an acceptor material (electron-accepting material), or a stacked-layer structure in which a layer containing a hole-transport material and a layer containing an acceptor material (electron-accepting material) are stacked.

The hole-transport layer 722 transports the holes, which are injected from the conductor 772 by the hole-injection layer 721, to the light-emitting layer 723. Note that the hole-transport layer 722 contains a hole-transport material. It is preferable that the HOMO level of the hole-transport material used for the hole-transport layer 722 be equal or close to that of the hole-injection layer 721, in particular.

Examples of the acceptor material used for the hole-injection layer 721 include oxides of a metal belonging to any of Group 4 to Group 8 of the periodic table. Specific examples include molybdenum oxide, vanadium oxide, niobium oxide, tantalum oxide, chromium oxide, tungsten oxide, manganese oxide, and rhenium oxide. Among these oxides, especially, molybdenum oxide is preferable since it is stable in the air, has a low hygroscopic property, and is easy to handle. Alternatively, organic acceptors such as a quinodimethane derivative, a chloranil derivative, and a hexaazatriphenylene derivative can be used.

The hole-transport materials used for the hole-injection layer 721 and the hole-transport layer 722 are preferably substances with a hole mobility of greater than or equal to 10⁻⁶ cm²/Vs. Note that other substances can also be used as long as the substances have a hole-transport property higher than an electron-transport property.

As the hole-transport material, a π-electron rich heteroaromatic compound (e.g., a carbazole derivative or an indole derivative), an aromatic amine compound, or the like can be favorably used. As the hole-transport material, a compound having an aromatic amine skeleton, a compound having a carbazole skeleton, a compound having a thiophene skeleton, a compound having a furan skeleton, or the like can be used. In addition, as the hole-transport material, a high molecular compound can be used.

Note that the hole-transport material is not limited to the above examples, and one of various known materials or a combination of some of various known materials can be used as the hole-transport material for the hole-injection layer 721 and the hole-transport layer 722. Note that the hole-transport layer 722 may be formed of a plurality of layers. That is, for example, the hole-transport layer 722 may have a stacked-layer structure of a first hole-transport layer and a second hole-transport layer.

<<Light-Emitting Layer 723>>

The light-emitting layer 723 is a layer containing a light-emitting substance. As the light-emitting substance, a substance whose emission color is blue, violet, bluish violet, green, yellowish green, yellow, orange, red, or the like is appropriately used. Here, when the light-emitting device 572 includes a plurality of EL layers as illustrated in FIG. 37C, FIG. 37D, and (E), the use of different light-emitting substances for the light-emitting layers 723 in the EL layers enables different emission colors to be exhibited (e.g., it enables white light emission obtained by combining complementary emission colors). For example, when the light-emitting device 572 has the structure illustrated in FIG. 37C, the use of different light-emitting substances for the light-emitting layer 723 in the EL layer 786a and the light-emitting layer 723 in the EL layer 786b can achieve different emission colors of the EL layer 786a and the EL layer 786b. Note that a stacked-layer structure in which one light-emitting layer contains different light-emitting substances may be employed.

The light-emitting layer 723 may contain one or more kinds of organic compounds (a host material and an assist material) in addition to a light-emitting substance (guest material). As one or more kinds of the organic compounds, one or both of the hole-transport material and the electron-transport material can be used.

When the light-emitting device 572 has the structure illustrated in FIG. 37C, it is preferred that a light-emitting substance that emits blue light (a blue-light-emitting substance) be used as a guest material in one of the EL layers 786a and 786b and a substance that emits green light (a green-light-emitting substance) and a substance that emits red light (a red-light-emitting substance) be used in the other EL layer. This structure is effective when the blue-light-emitting substance (blue-light-emitting layer) has lower light emission efficiency or a shorter lifetime than the others. Here, it is preferred that a light-emitting substance that converts singlet excitation energy into light in the visible light range be used as the blue-light-emitting substance and light-emitting substances that convert triplet excitation energy into light in the visible light range be used as the green- and red-light-emitting substances, whereby the spectrum balance between R, G, and B is improved.

There is no particular limitation on the light-emitting substance that can be used for the light-emitting layer 723, and it is possible to use a light-emitting substance that converts singlet excitation energy into light in the visible light range or a light-emitting substance that converts triplet excitation energy into light in the visible light range. Examples of the light-emitting substance are given below.

Examples of the light-emitting substance that converts singlet excitation energy into light include substances that exhibit fluorescence (fluorescent materials). Specific examples include a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. The pyrene derivative is a compound effective for meeting the chromaticity of blue in one embodiment of the present invention.

Examples of the light-emitting substance that converts triplet excitation energy into light include a substance that exhibits phosphorescence (phosphorescent material) and a thermally activated delayed fluorescence (TADF) material that exhibits thermally activated delayed fluorescence.

Examples of a phosphorescent material include an organometallic complex, a metal complex (platinum complex), and a rare earth metal complex. These substances exhibit the respective different emission colors (emission peaks) and thus, any of them is selected appropriately as needed.

Examples of a phosphorescent material which emits blue or green light and whose emission spectrum has a peak wavelength at greater than or equal to 450 nm and less than or equal to 570 nm include an organometallic complex having a 4H-triazole skeleton, an organometallic complex having a 1H-triazole skeleton, an organometallic complex having an imidazole skeleton, and an organometallic complex in which a phenylpyridine derivative having an electron-withdrawing group is a ligand.

Examples of a phosphorescent material which emits green or yellow light and whose emission spectrum has a peak wavelength at greater than or equal to 495 nm and less than or equal to 590 nm include an organometallic iridium complex having a pyrimidine skeleton, an organometallic iridium complex having a pyrazine skeleton, an organometallic iridium complex having a pyridine skeleton, an organometallic complex and a rare earth metal complex.

Among the above, organometallic iridium complexes having a pyridine skeleton (particularly, a phenylpyridine skeleton) or a pyrimidine skeleton are compounds effective for meeting the chromaticity of green in one embodiment of the present invention.

Examples of a phosphorescent material which emits yellow or red light and whose emission spectrum has a peak wavelength at greater than or equal to 570 nm and less than or equal to 750 nm include an organometallic complex having a pyrimidine skeleton, an organometallic complex having a pyrazine skeleton, an organometallic complex having a pyridine skeleton, a platinum complex, and a rare earth metal complex.

Among the above, organometallic iridium complexes having a pyrazine skeleton are compounds effective for meeting the chromaticity of red in one embodiment of the present invention. In particular, organometallic iridium complexes having a cyano group (e.g., [Ir(dmdppr-dmCP)₂(dpm)]) are preferable because of their high stability.

Note that as the blue-light-emitting substance, a substance whose photoluminescence peak wavelength is greater than or equal to 430 nm and less than or equal to 470 nm, preferably greater than or equal to 430 nm and less than or equal to 460 nm can be used. As the green-light-emitting substance, a substance whose photoluminescence peak wavelength is greater than or equal to 500 nm and less than or equal to 540 nm, preferably greater than or equal to 500 nm and less than or equal to 530 nm can be used. As the red-light-emitting substance, a substance whose photoluminescence peak wavelength is greater than or equal to 610 nm and less than or equal to 680 nm, preferably greater than or equal to 620 nm and less than or equal to 680 nm is used. Note that the photoluminescence may be measured with either a solution or a thin film.

With the parallel use of such compounds and the microcavity effect, the above chromaticity can be achieved more easily. Here, a transflective electrode (a metal thin film portion) that is needed for obtaining the microcavity effect has a thickness of preferably greater than or equal to 20 nm and less than or equal to 40 nm, further preferably greater than 25 nm and less than or equal to 40 nm. Note that the thickness greater than 40 nm possibly reduces the efficiency.

As the organic compounds (the host material and the assist material) used in the light-emitting layer 723, one or more kinds of substances having a larger energy gap than the light-emitting substance (the guest material) can be used. Note that the hole-transport materials listed above and the electron-transport materials given below can be used as the host material and the assist material, respectively.

When the light-emitting substance is a fluorescent material, it is preferable to use, as the host material, an organic compound that has a high energy level in a singlet excited state and has a low energy level in a triplet excited state. For example, an anthracene derivative or a tetracene derivative is preferably used.

When the light-emitting substance is a phosphorescent material, an organic compound having triplet excitation energy (energy difference between a ground state and a triplet excited state) higher than that of the light-emitting substance can be selected as the host material. In that case, it is possible to use a zinc- or aluminum-based metal complex, an oxadiazole derivative, a triazole derivative, a benzimidazole derivative, a quinoxaline derivative, a dibenzoquinoxaline derivative, a dibenzothiophene derivative, a dibenzofuran derivative, a pyrimidine derivative, a triazine derivative, a pyridine derivative, a bipyridine derivative, a phenanthroline derivative, an aromatic amine, a carbazole derivative, or the like.

When a plurality of organic compounds are used for the light-emitting layer 723, compounds that form an exciplex are preferably mixed with a light-emitting substance. In that case, any of various organic compounds can be used in an appropriate combination; to form an exciplex efficiently, it is particularly preferable to combine a compound that easily accepts holes (hole-transport material) and a compound that easily accepts electrons (electron-transport material). As the hole-transport material and the electron-transport material, specifically, any of the materials described in this embodiment can be used.

The TADF material enables up-conversion of a triplet excited state into a singlet excited state (i.e., reverse intersystem crossing) using a little thermal energy and efficiently emits light from the singlet excited state (efficiently exhibits fluorescence). Thermally activated delayed fluorescence is efficiently obtained under the condition where the energy difference between the triplet excitation level and the singlet excitation level is greater than or equal to 0 eV and less than or equal to 0.2 eV, preferably greater than or equal to 0 eV and less than or equal to 0.1 eV. Note that delayed fluorescence exhibited by the TADF material refers to light emission having the same spectrum as normal fluorescence and an extremely long lifetime. The lifetime is 1×10⁻⁶ seconds or longer, preferably 1×10⁻³ seconds or longer.

Examples of the TADF material include fullerene, a derivative thereof, an acridine derivative such as proflavine, and eosin. Other examples include a metal-containing porphyrin such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP). As the TADF material, a heterocyclic compound including a π-electron rich heteroaromatic ring and a π-electron deficient heteroaromatic ring can also be used. Note that a substance in which a π-electron rich heteroaromatic ring is directly bonded to a π-electron deficient heteroaromatic ring is particularly preferable because both the donor property of the π-electron rich heteroaromatic ring and the acceptor property of the π-electron deficient heteroaromatic ring are improved and the energy difference between the singlet excited state and the triplet excited state becomes small.

Note that a TADF material can also be used in combination with another organic compound.

<<Electron-Transport Layer 724>>

The electron-transport layer 724 transports electrons, which are injected from the conductor 788 by the electron-injection layer 725, to the light-emitting layer 723. Note that the electron-transport layer 724 contains an electron-transport material. The electron-transport material used for the electron-transport layer 724 is preferably a substance with an electron mobility of higher than or equal to 1×cm²/Vs. Note that any other substance can also be used as long as the substance transports electrons more easily than it transports holes.

Examples of the electron-transport material include metal complexes having a quinoline ligand, a benzoquinoline ligand, an oxazole ligand, and a thiazole ligand; an oxadiazole derivative; a triazole derivative; a phenanthroline derivative; a pyridine derivative; and a bipyridine derivative. In addition, a π-electron deficient heteroaromatic compound such as a nitrogen-containing heteroaromatic compound can also be used.

The electron-transport layer 724 is not limited to a single layer and may be a stack of two or more layers each containing any of the above substances.

<<Electron-Injection Layer 725>>

The electron-injection layer 725 contains a substance having a high electron-injection property. The electron-injection layer 725 can be formed using an alkali metal, an alkaline earth metal, or a compound thereof, such as lithium fluoride (LiF), cesium fluoride (CsF), calcium fluoride (CaF₂), or lithium oxide (LiOₓ). A rare earth metal compound like erbium fluoride (ErF₃) can also be used. An electride may also be used for the electron-injection layer 725. An example of the electride includes a substance in which electrons are added at high concentration to calcium oxide-aluminum oxide. Any of the above-described substances used for the electron-transport layer 724 can also be used.

A composite material in which an organic compound and an electron donor (donor) are mixed may also be used for the electron-injection layer 725. Such a composite material is excellent in an electron-injection property and an electron-transport property because electrons are generated in the organic compound by the electron donor. The organic compound here is preferably a material excellent in transporting the generated electrons; specifically, for example, the electron-transport material used for the electron-transport layer 724 (e.g., a metal complex or a heteroaromatic compound) can be used. As the electron donor, a substance showing an electron-donating property with respect to the organic compound is used. Specifically, an alkali metal, an alkaline earth metal, and a rare earth metal are preferable, and lithium, cesium, magnesium, calcium, erbium, ytterbium, and the like are given. In addition, an alkali metal oxide and an alkaline earth metal oxide are preferable, and lithium oxide, calcium oxide, barium oxide, and the like are given. Alternatively, a Lewis base such as magnesium oxide can be used. Further alternatively, an organic compound such as tetrathiafulvalene (abbreviation: TTF) can be used.

<<Charge Generation Layer 792>>

The charge generation layer 792 has a function of injecting electrons into the EL layer 786 that is closer to the conductor 772 of the two EL layers 786 in contact with the charge generation layer 792 and injecting holes to the other EL layer 786 that is closer to the conductor 788, when a voltage is applied between the conductor 772 and the conductor 788. For example, in the light-emitting device 572 having the structure illustrated in FIG. 37C, the charge generation layer 792 has a function of injecting electrons into the EL layer 786a and injecting holes into the EL layer 786b. Note that the charge generation layer 792 may have either a structure in which an electron acceptor (acceptor) is added to a hole-transport material or a structure in which an electron donor (donor) is added to an electron-transport material. Alternatively, both of these structures may be stacked. Forming the charge generation layer 792 by using any of the above materials can inhibit the increase in driving voltage of the display device 810 including the stack of the EL layers.

When the charge generation layer 792 has a structure in which an electron acceptor is added to a hole-transport material, the electron acceptor can be 7,7,8,8-tetracyano-2,3,5,6-tetrafluoroquinodimethane (abbreviation: F₄-TCNQ), chloranil, or the like. Other examples include oxides of metals that belong to Group 4 to Group 8 of the periodic table. Specific examples are vanadium oxide, niobium oxide, tantalum oxide, chromium oxide, molybdenum oxide, tungsten oxide, manganese oxide, and rhenium oxide.

When the charge generation layer 792 has a structure in which an electron donor is added to an electron-transport material, an alkali metal, an alkaline earth metal, a rare earth metal, or a metal that belongs to Group 2 or Group 13 of the periodic table, or an oxide or carbonate thereof can be used as the electron donor. Specifically, lithium (Li), cesium (Cs), magnesium (Mg), calcium (Ca), ytterbium (Yb), indium (In), lithium oxide, cesium carbonate, or the like is preferably used. An organic compound such as tetrathianaphthacene may be used as the electron donor.

For fabrication of the light-emitting device 572, a vacuum process such as an evaporation method or a solution process such as a spin coating method or an ink-jet method can be used. In the case of employing an evaporation method, it is possible to use a physical vapor deposition method (PVD method) such as a sputtering method, an ion plating method, an ion beam evaporation method, a molecular beam evaporation method, or a vacuum evaporation method, a chemical vapor deposition method (CVD method), or the like. Specifically, the functional layers (the hole-injection layer, the hole-transport layer, the light-emitting layer, the electron-transport layer, and the electron-injection layer) included in the EL layer and the charge generation layer of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, screen printing (stencil), offset printing (planography), flexography (relief printing), gravure printing, or micro-contact printing), or the like.

Note that materials that can be used for the functional layers (the hole-injection layer, the hole-transport layer, the light-emitting layer, the electron-transport layer, and the electron-injection layer) included in the EL layer and the charge generation layer of the light-emitting device described in this embodiment are not limited to the above materials, and other materials can be used in combination as long as the a function of the layers are fulfilled. For example, a high molecular compound (e.g., an oligomer, a dendrimer, and a polymer), a middle molecular compound (a compound between a low molecular compound and a high molecular compound, with a molecular weight of 400 to 4000), or an inorganic compound (e.g., a quantum dot material) can be used. The quantum dot material may be a colloidal quantum dot material, an alloyed quantum dot material, a core-shell quantum dot material, a core quantum dot material, or the like.

The display device 810 described in this embodiment can be used for the light source included in the detection device 17 described in Embodiment 1. With the use of the display device 810 for the light source described in Embodiment 1, the light-sources can be arranged at high density. Thus, the electronic device of one embodiment of the present invention can obtain information of the user of the electronic device with a high accuracy.

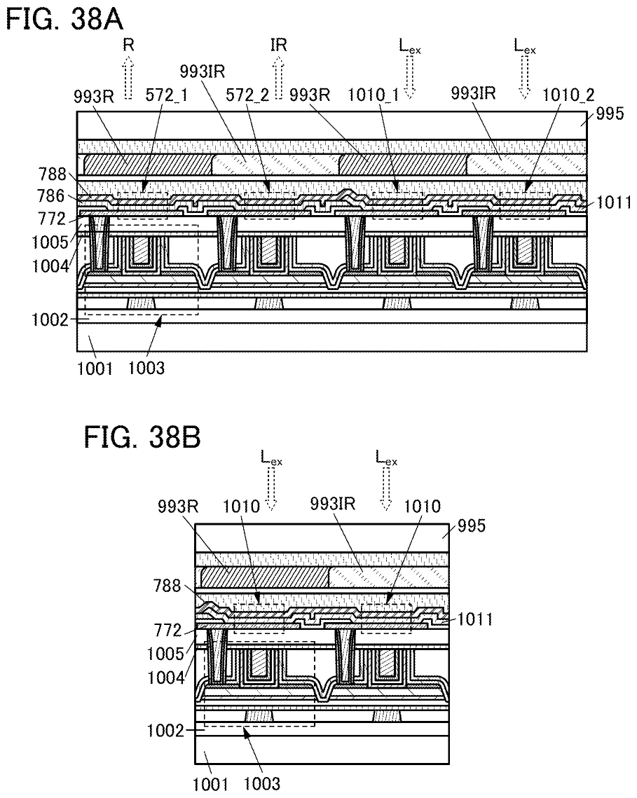

FIG. 38A illustrates a structure example of an imaging device that can be used for the detection device 17 described in Embodiment 1. FIG. 38A is a cross-sectional view illustrating a structure of the imaging device. As illustrated in FIG. 38A, a transistor 1003, the light-emitting device 572, a photoelectric conversion device 1010, a coloring layer 993R, a coloring layer 99318 and the like can be provided between a substrate 1001 and the substrate 995. Here, the transistor 1003 can be an OS transistor, for example. FIG. 38A illustrates four transistors 1003.

An insulator 1002 is provided over the substrate 1001, and the transistor 1003 is provided over the insulator 1002. An insulator 1004 is provided over the transistor 1003, and an insulator 1005 is provided over the insulator 1004. The light-emitting device 572 and the photoelectric conversion device 1010 are provided over the insulator 1005. The coloring layer 993R and the coloring layer 9931R are provided to have a region overlapping with the light-emitting device 572 or the photoelectric conversion device 1010. FIG. 38A shows that two light-emitting devices 572 (a light-emitting device 572_1 and a light-emitting device 572_2) and two photoelectric conversion devices 1010 (a photoelectric conversion device 1010_1 and a photoelectric conversion device 1010_2) are electrically connected to the respective different transistors 1003. In FIG. 38A, the coloring layer 993R having a function of transmitting red light is provided to have a region overlapping with the light-emitting device 572_1 and the coloring layer 99318 having a function of transmitting infrared light is provided to have a region overlapping with the light-emitting device 572_2. In addition, the coloring layer 993R is provided to have a region overlapping with the photoelectric conversion device 1010_1, and the coloring layer 9931R is provided to have a region overlapping with the photoelectric conversion device 1010_2.

The photoelectric conversion device 1010 has a function of receiving light Lₑₓ that comes from the outside of the imaging device and converting it into an electric signal corresponding to the illuminance of the received light Lₑₓ.

The light-emitting device 572 preferably has a function of emitting white light and infrared light. Accordingly, light emitted from the light-emitting device 572_1 is emitted to the outside of the imaging device through the coloring layer 993R as red light R. Light emitted from the light-emitting device 572_2 is emitted to the outside of the imaging device through the coloring layer 9931R as infrared light IR. The red light R and the infrared light IR, which are emitted to the outside of the imaging device, strike an object and are reflected and applied to the photoelectric conversion devices 1010. For example, when the imaging device having the structure illustrated in FIG. 38A is used for the glasses-type electronic device described in Embodiment 1, the red light R and the infrared light IR are delivered to the face of the user of the glasses-type electronic device, and the reflected light Lₑₓ can be detected by the photoelectric conversion devices 1010.

By having the function of detecting both red light and infrared light, the imaging device can detect, for example, the state of the eyes and surrounding areas of the user of the electronic device of one embodiment of the present invention can be detected with a higher accuracy than an imaging device having a function of detecting only one of red light and infrared light. Consequently, a facial feature of the user of the electronic device of one embodiment of the present invention, such as the user's facial expression, can be recognized with a high accuracy, for example; thus, the electronic device of one embodiment of the present invention can have a function of estimating the degree of fatigue or emotions of the user with a high accuracy, for instance.

Note that when the display device of one embodiment of the present invention includes a photoelectric conversion device, the display device can have the structure illustrated in FIG. 38A. In this case, the display device includes the light-emitting device 572 including a region overlapping with the coloring layer 993R having a function of transmitting red light, the light-emitting device 572 including a region overlapping with the coloring layer 99318 having a function of transmitting infrared light, the light-emitting device 572 including a region overlapping with a coloring layer having a function of transmitting green light, and the light-emitting device 572 including a region overlapping with a coloring layer having a function of transmitting blue light.

The light-emitting device 572 is composed of the conductor 772, the EL layer 786, and the conductor 788. The photoelectric conversion device 1010 is composed of the conductor 772, an active layer 1011, and the conductor 788. The transistor 1003 is electrically connected to the conductor 772.

The active layer 1011 can have a stacked-layer structure in which a p-type semiconductor and an n-type semiconductor are stacked to form a PN junction; or a stacked-layer structure in which a p-type semiconductor, an i-type semiconductor, and an n-type semiconductor are stacked to form a PIN junction, for example.

As the semiconductor used for the active layer 1011, an inorganic semiconductor such as silicon or an organic semiconductor containing an organic compound can be used. In particular, the use of an organic semiconductor material is preferable, in which case the EL layer 786 of the light-emitting device 572 and the active layer 1011 are easily formed by the same vacuum evaporation method, and thus the same manufacturing apparatus can be used.

When an organic semiconductor material is used for the active layer 1011, an electron-accepting organic semiconductor material such as fullerene (e.g., C₆₀ or C₇₀) or its derivative can be used as an n-type semiconductor material. As a p-type semiconductor material, an electron-donating organic semiconductor material such as copper(II) phthalocyanine (CuPc) or tetraphenyldibenzoperiflanthene (DBP) can be used. The active layer 1011 may have a stacked-layer structure (a P-N structure) including an electron-accepting semiconductor material and an electron-donating semiconductor material, or a stacked-layer structure (a P-I-N structure) in which a bulk heterostructure layer formed by co-evaporation of an electron-accepting semiconductor material and an electron-donating semiconductor material is provided between the materials of the P-N structure. Furthermore, a layer functioning as a hole blocking layer or a layer functioning as an electron blocking layer may be provided around (above or below) the P-N structure or the P-I-N structure, in order to inhibit dark current without light illumination.

In the light-emitting device 572, the EL layer 786 is provided over the conductor 772. In the photoelectric conversion device 1010, the active layer 1011 is provided over the conductor 772. The conductor 788 is provided to cover the EL layer 786 and the active layer 1011. Accordingly, the conductor 788 can serve as both the electrode of the light-emitting device 572 and the electrode of the photoelectric conversion device 1010.

FIG. 38B is a cross-sectional view illustrating a structure example of the imaging device of one embodiment of the present invention, and illustrates a variation example of the structure in FIG. 38A. The imaging device having the structure in FIG. 38B is different from the imaging device having the structure in FIG. 38A in that the light-emitting device 572 is not provided.

When the electronic device of one embodiment of the present invention includes the imaging device having the structure in FIG. 38B, providing a light source outside the imaging device allows the imaging device to detect light emitted from the light source. For example, when the imaging device having the structure in FIG. 38B is used for the glasses-type electronic device described in Embodiment 1, red light and infrared light that are emitted from the light source are delivered to the face of the user of the glasses-type electronic device, and the reflected light Lₑₓ can be detected by the photoelectric conversion devices 1010.

When the imaging device included in the electronic device of one embodiment of the present invention has the structure illustrated in FIG. 38B, the photoelectric conversion devices 1010 can be provided at high density in the imaging device.

At least part of any of the structure examples, the drawings corresponding thereto, and the like described in this embodiment can be implemented in combination with any of the other structure examples, the other drawings, and the like as appropriate.

At least part of this embodiment can be implemented in combination with any of the other embodiments described in this specification as appropriate.

### Embodiment 3

In this embodiment, transistors that can be used in the display device of one embodiment of the present invention will be described.

### Structure Example 1 of Transistor

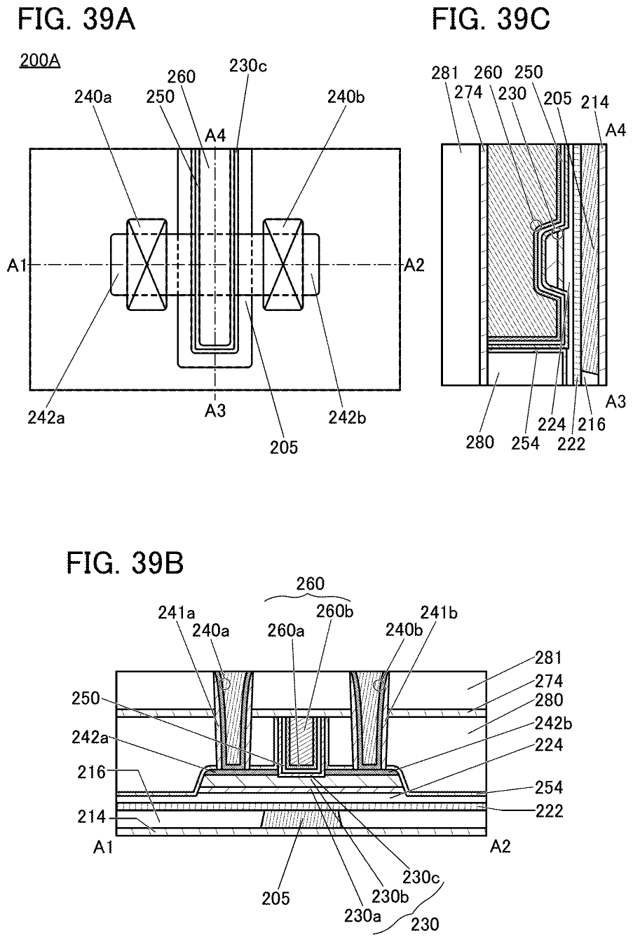

FIG. 39A, FIG. 39B, and (C) are a top view and cross-sectional views of a transistor 200A that can be used in the display device of one embodiment of the present invention, and the periphery of the transistor 200A. The transistor 200A can be used as the transistors included in the pixel array 833, the gate driver circuit 821, the source driver circuit 822, and the circuit 840 described in Embodiment 1 and the like.

FIG. 39A is a top view of the transistor 200A. FIG. 39B and FIG. 39C are cross-sectional views of the transistor 200A. FIG. 39B is a cross-sectional view taken along the dashed-dotted line A1-A2 in FIG. 39A and shows a cross section of the transistor 200A in the channel length direction. FIG. 39C is a cross-sectional view taken along the dashed-dotted line A3-A4 in FIG. 39A and shows a cross section of the transistor 200A in the channel width direction. Note that for simplification of the drawing, some components are not illustrated in the top view in FIG. 39A.

The transistor 200A includes a metal oxide 230a over a substrate (not illustrated); a metal oxide 230b over the metal oxide 230a; a conductor 242a and a conductor 242b that are apart from each other over the metal oxide 230b; the insulator 280 that is positioned over the conductor 242a and the conductor 242b and has an opening between the conductor 242a and the conductor 242b; a conductor 260 in the opening; an insulator 250 between the conductor 260 and the metal oxide 230b, the conductor 242a, the conductor 242b, and the insulator 280; and a metal oxide 230c between the insulator 250 and the metal oxide 230b, the conductor 242a, the conductor 242b, and the insulator 280. Here, as illustrated in FIG. 39B and FIG. 39C, the top surface of the conductor 260 is substantially aligned with the top surfaces of the insulator 250, the insulator 254, the metal oxide 230c, and the insulator 280. Hereinafter, the metal oxide 230a, the metal oxide 230b, and the metal oxide 230c may be collectively referred to as a metal oxide 230. The conductor 242a and the conductor 242b may be collectively referred to as a conductor 242 in some cases.

As illustrated in FIG. 39B, in the transistor 200A, the side surfaces of the conductor 242a and the conductor 242b closer to the conductor 260 are substantially perpendicular. Note that the transistor 200A illustrated in FIG. 39 is not limited thereto, and the angle formed between the side surface and the bottom surface of each of the conductor 242a and the conductor 242b may range from 10° to 80°, inclusive, preferably from 30° to 60°, inclusive. The facing side surfaces of the conductor 242a and the conductor 242b may each have a plurality of surfaces.

As illustrated in FIG. 39B and FIG. 39C, the insulator 254 is preferably provided between the insulator 280 and the insulator 224, the metal oxide 230a, the metal oxide 230b, the conductor 242a, the conductor 242b, and the metal oxide 230c. Here, as illustrated in FIG. 39B and FIG. 39C, the insulator 254 preferably includes a region in contact with the side surface of the metal oxide 230c, the top surface and side surface of the conductor 242a, the top surface and side surface of the conductor 242b, the side surface of the metal oxide 230a, the side surface of the metal oxide 230b, and the top surface of the insulator 224.

In the transistor 200A, three layers of the metal oxide 230a, the metal oxide 230b, and the metal oxide 230c are stacked in and around the region where the channel is formed (hereinafter also referred to as channel formation region); however, the present invention is not limited thereto. For example, a two-layer structure of the metal oxide 230b and the metal oxide 230c or a stacked-layer structure of four or more layers may be employed. Although the conductor 260 has a stacked-layer structure of two layers in the transistor 200A, the present invention is not limited thereto. For example, the conductor 260 may have a single-layer structure or a stacked-layer structure of three or more layers. Furthermore, each of the metal oxide 230a, the metal oxide 230b, and the metal oxide 230c may have a stacked-layer structure of two or more layers.

For example, when the metal oxide 230c has a stacked-layer structure including a first metal oxide and a second metal oxide over the first metal oxide, the first metal oxide preferably has a composition similar to that of the metal oxide 230b and the second metal oxide preferably has a composition similar to that of the metal oxide 230a.

Here, the conductor 260 functions as a gate electrode of the transistor, and the conductor 242a and the conductor 242b function as a source electrode and a drain electrode. As described above, the conductor 260 is formed to be embedded in the opening of the insulator 280 and the region between the conductor 242a and the conductor 242b. Here, the positions of the conductor 260, the conductor 242a, and the conductor 242b with respect to the opening of the insulator 280 are selected in a self-aligned manner. That is, in the transistor 200A, the gate electrode can be positioned between the source electrode and the drain electrode in a self-aligned manner. Thus, the conductor 260 can be formed without an alignment margin, resulting in a reduction in the footprint of the transistor 200A. Consequently, a display device can achieve high resolution and have a narrow bezel.

In addition, as illustrated in FIG. 39, the conductor 260 preferably includes a conductor 260a provided inside the insulator 250 and a conductor 260b embedded inside the conductor 260a.

As illustrated in FIG. 39A, FIG. 39B, and FIG. 39C, the transistor 200A preferably includes the insulator 214 over the substrate (not illustrated); the insulator 216 over the insulator 214; a conductor 205 embedded in the insulator 216; the insulator 222 over the insulator 216 and the conductor 205; and the insulator 224 over the insulator 222. The metal oxide 230a is preferably positioned over the insulator 224.

The insulator 274 and the insulator 281 functioning as interlayer films are preferably provided over the transistor 200A. Here, the insulator 274 is preferably provided in contact with the top surfaces of the conductor 260, the insulator 250, the insulator 254, the metal oxide 230c, and the insulator 280.

The insulator 222, the insulator 254, and the insulator 274 preferably have a function of inhibiting diffusion of hydrogen (e.g., at least one of hydrogen atoms and hydrogen molecules). For example, the insulator 222, the insulator 254, and the insulator 274 preferably have a lower hydrogen permeability than the insulator 224, the insulator 250, and the insulator 280. Moreover, the insulator 222 and the insulator 254 preferably have a function of inhibiting diffusion of oxygen (e.g., at least one of oxygen atoms and oxygen molecules). For example, the insulators 222 and 254 preferably have a lower oxygen permeability than the insulator 224, the insulator 250, and the insulator 280.

Here, the insulator 224, the metal oxide 230, and the insulator 250 are separated from the insulator 280 and the insulator 281 by the insulator 254 and the insulator 274. This can inhibit entry of impurities such as hydrogen included in the insulator 280 and the insulator 281 and excess oxygen into the insulator 224, the metal oxide 230, and the insulator 250.

A conductor 240 (a conductor 240a and a conductor 240b) that is electrically connected to the transistor 200A and functions as a plug is preferably provided. Note that an insulator 241 (an insulator 241a and an insulator 241b) is provided in contact with the side surface of the conductor 240 functioning as a plug. In other words, the insulator 241 is provided in contact with the inner wall of an opening in the insulator 254, the insulator 280, the insulator 274, and the insulator 281. Alternatively, a first conductor of the conductor 240 may be provided in contact with the side surface of the insulator 241 and a second conductor of the conductor 240 may be provided on the inner side of the first conductor. Here, the top surface of the conductor 240 and the top surface of the insulator 281 can be at substantially the same level. Although the first conductor of the conductor 240 and the second conductor of the conductor 240 are stacked in the transistor 200A, the present invention is not limited thereto. For example, the conductor 240 may have a single-layer structure or a stacked-layer structure of three or more layers. In the case where a stacked-layer structure is employed, the layers are sometimes distinguished by numbers corresponding to the formation order.

In the transistor 200A, a metal oxide functioning as an oxide semiconductor (hereinafter such a metal oxide is also referred to as an oxide semiconductor) is preferably used for the metal oxide 230 including the channel formation region (the metal oxide 230a, the metal oxide 230b, and the metal oxide 230c). For example, the metal oxide to be the channel formation region of the metal oxide 230 has a band gap of preferably 2 eV or higher, further preferably 2.5 eV or higher, as described above.

As illustrated in FIG. 39B, the metal oxide 230b may have a smaller thickness in a region that is not overlapped with the conductor 242 than in a region overlapped with the conductor 242. The thin region is formed when part of the top surface of the metal oxide 230b is removed at the time of forming the conductor 242a and the conductor 242b. When a conductive film to be the conductor 242 is formed, a low-resistance region may be formed on the top surface of the metal oxide 230b in the vicinity of the interface with the conductive film. Removing the low-resistance region between the conductor 242a and the conductor 242b on the top surface of the metal oxide 230b in the above manner can inhibit formation of the channel in the region.

According to one embodiment of the present invention, a display device that includes small-size transistors and has high resolution can be provided. A display device that includes transistors with a high on-state current and achieves high luminance can be provided. A display device that includes fast-response transistors and operates at high speed can be provided. A display device that includes transistors having stable electrical characteristics and is highly reliable can be provided. A display device that includes transistors with a low off-state current and achieves low power consumption can be provided.

The structure of the transistor 200A that can be used in the display device of one embodiment of the present invention will be described in detail.

The conductor 205 is placed so as to include a region overlapped by the metal oxide 230 and the conductor 260. The conductor 205 is preferably embedded in the insulator 216. Here, the top surface of the conductor 205 preferably has favorable planarity. For example, the average surface roughness (Ra) of the top surface of the conductor 205 is less than or equal to 1 nm, preferably less than or equal to 0.5 nm, further preferably less than or equal to 0.3 nm. This achieves favorable planarity of the insulator 224 formed over the conductor 205 and increases the crystallinity of the metal oxide 230b and the metal oxide 230c.

Here, the conductor 260 functions as a first gate (also referred to as top gate) electrode in some cases. The conductor 205 functions as a second gate (also referred to back gate) electrode in some cases. In that case, by changing a potential applied to the conductor 205 independently of a potential applied to the conductor 260, Vₜₕ of the transistor 200A can be controlled. In particular, by applying a negative potential to the conductor 205, Vₜₕ of the transistor 200A can be higher than 0 V, and its off-state current can be reduced. Thus, a drain current of the transistor 200A at the time when a potential applied to the conductor 260 is 0 V can be smaller in the case where a negative potential is applied to the conductor 205 than in the case where the negative potential is not applied to the conductor 205.

The conductor 205 is preferably larger than the channel formation region of the metal oxide 230. It is particularly preferred that the conductor 205 extend beyond an end portion of the metal oxide 230 that intersects with the channel width direction, as illustrated in FIG. 39C. That is, the conductor 205 and the conductor 260 preferably overlap each other with the insulator positioned therebetween, in a region beyond the side surface of the metal oxide 230 in the channel width direction.

With the above structure, the channel formation region of the metal oxide 230 can be electrically surrounded by electric fields of the conductor 260 functioning as the first gate electrode and electric fields of the conductor 205 functioning as the second gate electrode.

As illustrated in FIG. 39C, the conductor 205 is extended to have a function of a wiring. However, without limitation to this structure, a conductor functioning as a wiring may be provided under the conductor 205.

A conductive material containing tungsten, copper, or aluminum as its main component is preferably used for the conductor 205. Note that the conductor 205 is shown as a single layer but may have a stacked-layer structure, for example, a stack of titanium or titanium nitride and any of the above conductive materials.

In addition, a conductor having a function of inhibiting diffusion of impurities such as a hydrogen atom, a hydrogen molecule, a water molecule, a nitrogen atom, a nitrogen molecule, a nitrogen oxide molecule (e.g., N₂O, NO, and NO₂), and a copper atom (a conductor through which the above impurities are less likely to pass) may be provided under the conductor 205.

Alternatively, it is preferable to provide a conductor having a function of inhibiting diffusion of oxygen (e.g., at least one of oxygen atoms and oxygen molecules) (a conductor through which oxygen is less likely to pass). Note that in this specification, a function of inhibiting diffusion of impurities or oxygen means a function of inhibiting diffusion of any one or all of the above impurities and oxygen.

When the conductor having a function of inhibiting oxygen diffusion is provided under the conductor 205, a reduction in conductivity of the conductor 205 due to oxidation of the conductor 205 can be inhibited. As the conductor having a function of inhibiting oxygen diffusion, tantalum, tantalum nitride, ruthenium, or ruthenium oxide is preferably used, for example. The conductor 205 can therefore be a single layer or a stack of the above conductive materials.

The insulator 214 preferably functions as a barrier insulating film for inhibiting impurities such as water or hydrogen from entering the transistor 200A from the substrate side. Accordingly, the insulator 214 is preferably formed using an insulating material having a function of inhibiting diffusion of impurities such as a hydrogen atom, a hydrogen molecule, a water molecule, a nitrogen atom, a nitrogen molecule, a nitrogen oxide molecule (e.g., N₂O, NO, and NO₂), and a copper atom (an insulating material through which the above impurities are less likely to pass). Alternatively, the insulator 214 is preferably formed using an insulating material having a function of inhibiting diffusion of oxygen (e.g., at least one of oxygen atoms and oxygen molecules) (an insulating material through which oxygen is less likely to pass).

For example, aluminum oxide or silicon nitride is preferably used for the insulator 214. Accordingly, it is possible to inhibit diffusion of impurities such as water or hydrogen into the transistor 200A from the substrate side through the insulator 214. It is also possible to inhibit diffusion of oxygen contained in the insulator 224 and the like toward the substrate through the insulator 214.

The dielectric constant of each of the insulator 216, the insulator 280, and the insulator 281 functioning as interlayer films is preferably lower than that of the insulator 214. The use of a material having a low dielectric constant for the interlayer film can reduce the parasitic capacitance between wirings. For example, for the insulator 216, the insulator 280, and the insulator 281, silicon oxide, silicon oxynitride, silicon nitride oxide, silicon nitride, silicon oxide to which fluorine is added, silicon oxide to which carbon is added, silicon oxide to which carbon and nitrogen are added, porous silicon oxide, or the like is used as appropriate.

The insulator 222 and the insulator 224 function as a gate insulator.

Here, it is preferred that the insulator 224 in contact with the metal oxide 230 release oxygen by heating. In this specification and the like, oxygen that is released by heating is referred to as excess oxygen in some cases. For example, silicon oxide or silicon oxynitride can be used as appropriate for the insulator 224. When such an insulator containing oxygen is provided in contact with the metal oxide 230, oxygen vacancies in the metal oxide 230 can be reduced, leading to an improvement in reliability of the transistor 200A.

Specifically, an oxide material that releases some oxygen by heating is preferably used for the insulator 224. An oxide that releases oxygen by heating is an oxide film in which the amount of released oxygen converted into oxygen atoms is greater than or equal to 1.0×10¹⁸ atoms/cm³, preferably greater than or equal to 1.0×10¹⁹ atoms/cm³, further preferably greater than or equal to 2.0×10¹⁹ atoms/cm³ or greater than or equal to 3.0×10²⁰ atoms/cm³ in thermal desorption spectroscopy (TDS) analysis. Note that the temperature of the film surface in the TDS analysis is preferably higher than or equal to 100° C. and lower than or equal to 700° C., or higher than or equal to 100° C. and lower than or equal to 400° C.

As illustrated in FIG. 39C, the insulator 224 is sometimes thinner in a region overlapped with neither the insulator 254 nor the metal oxide 230b than in the other regions. In the insulator 224, the region overlapped with neither the insulator 254 nor the metal oxide 230b preferably has a thickness with which the above oxygen can be adequately diffused.

Like the insulator 214 and the like, the insulator 222 preferably functions as a barrier insulating film that inhibits entry of impurities such as water or hydrogen into the transistor 200A from the substrate side. For example, the insulator 222 preferably has a lower hydrogen permeability than the insulator 224. When the insulator 224, the metal oxide 230, the insulator 250, and the like are surrounded by the insulator 222, the insulator 254, and the insulator 274, entry of impurities such as water or hydrogen into the transistor 200A from the outside can be inhibited.

Furthermore, the insulator 222 preferably has a function of inhibiting diffusion of oxygen (e.g., at least one of oxygen atoms and oxygen molecules) (it is preferred that oxygen is less likely to pass through the insulator 222). For example, the insulator 222 preferably has a lower oxygen permeability than the insulator 224. The insulator 222 preferably has a function of inhibiting diffusion of oxygen and impurities, in which case oxygen contained in the metal oxide 230 is less likely to diffuse toward the substrate. The insulator 222 can also inhibit the conductor 205 from reacting with oxygen contained in the insulator 224 and oxygen contained in the metal oxide 230.

As the insulator 222, an insulator containing one or both of an oxide of aluminum and an oxide of hafnium, which are insulating materials, is preferably used. As the insulator containing one or both of an oxide of aluminum and an oxide of hafnium, aluminum oxide or hafnium oxide is preferably used. Alternatively, an oxide containing aluminum and hafnium (hafnium aluminate) or the like is preferably used. The insulator 222 formed using such a material functions as a layer inhibiting oxygen release from the metal oxide 230 and entry of impurities such as hydrogen into the metal oxide 230 from the periphery of the transistor 200A.

Alternatively, aluminum oxide, bismuth oxide, germanium oxide, niobium oxide, silicon oxide, titanium oxide, tungsten oxide, yttrium oxide, or zirconium oxide may be added to the insulator, for example. Alternatively, the insulator may be subjected to nitriding treatment. Silicon oxide, silicon oxynitride, or silicon nitride may be stacked over the insulator.

The insulator 222 may have a single-layer structure or a stacked-layer structure using an insulator containing a high-k material, such as aluminum oxide, hafnium oxide, tantalum oxide, zirconium oxide, lead zirconate titanate (PZT), strontium titanate (SrTiO₃), or (Ba,Sr)TiO₃ (BST). As miniaturization and high integration of transistors progress, a problem such as generation of leakage current may arise because of a thinner gate insulator. When a high-k material is used for an insulator functioning as the gate insulator, a gate potential at the time when the transistor operates can be lowered while the physical thickness is maintained.

Note that the insulator 222 and the insulator 224 may each have a stacked-layer structure of two or more layers. In that case, the stacked layers are not necessarily formed of the same material and may be formed of different materials. For example, an insulator similar to the insulator 224 may be provided below the insulator 222.

The metal oxide 230 includes the metal oxide 230a, the metal oxide 230b over the metal oxide 230a, and the metal oxide 230c over the metal oxide 230b. The metal oxide 230a under the metal oxide 230b inhibits diffusion of impurities into the metal oxide 230b from the components formed below the metal oxide 230a. The metal oxide 230c over the metal oxide 230b inhibits diffusion of impurities into the metal oxide 230b from the components formed above the metal oxide 230c.

Note that the metal oxide 230 preferably has a stacked-layer structure of oxide layers with different atomic ratios of metal atoms. Specifically, the atomic ratio of the element Mto the constituent elements in the metal oxide used as the metal oxide 230a is preferably higher than that in the metal oxide used as the metal oxide 230b. The atomic ratio of the element M to In in the metal oxide used as the metal oxide 230a is preferably higher than that in the metal oxide used as the metal oxide 230b. The atomic ratio of In to the element M in the metal oxide used as the metal oxide 230b is preferably higher than that in the metal oxide used as the metal oxide 230a. The metal oxide 230c can be formed using a metal oxide that can be used as the metal oxide 230a or the metal oxide 230b.

The metal oxide 230a, the metal oxide 230b, and the metal oxide 230c preferably have crystallinity, and are particularly preferably formed using CAAC-OS (c-axis-aligned crystalline oxide semiconductor). An oxide having crystallinity, such as a CAAC-OS, has a dense structure with small amounts of impurities and defects (e.g., oxygen vacancies) and high crystallinity. This reduces oxygen extraction from the metal oxide 230b by the source electrode or the drain electrode. Accordingly, oxygen extraction from the metal oxide 230b can be inhibited even when heat treatment is performed. Thus, the transistor 200A is stable against high temperatures in the manufacturing process (i.e., thermal budget).

The energy of the conduction band minimum of each of the metal oxide 230a and the metal oxide 230c is preferably higher than that of the metal oxide 230b. In other words, the electron affinity of each of the metal oxide 230a and the metal oxide 230c is preferably smaller than that of the metal oxide 230b. In that case, the metal oxide 230c is preferably formed using a metal oxide that can be used as the metal oxide 230a. Specifically, the atomic ratio of the element M to the constituent elements in the metal oxide used as the metal oxide 230c is preferably higher than that in the metal oxide used as the metal oxide 230b. The atomic ratio of the element M to In in the metal oxide used as the metal oxide 230c is preferably higher than that in the metal oxide used as the metal oxide 230b. The atomic ratio of In to the element M in the metal oxide used as the metal oxide 230b is preferably higher than that in the metal oxide used as the metal oxide 230c.

Here, the energy level of the conduction band minimum is gradually varied at junction portions of the metal oxide 230a, the metal oxide 230b, and the metal oxide 230c. In other words, the energy level of the conduction band minimum at junction portions of the metal oxide 230a, the metal oxide 230b, and the metal oxide 230c continuously vary or are continuously connected. This can be achieved by decrease in the density of defect states in a mixed layer formed at the interface between the metal oxide 230a and the metal oxide 230b and the interface between the metal oxide 230b and the metal oxide 230c.

Specifically, when the metal oxide 230a and the metal oxide 230b or the metal oxide 230b and the metal oxide 230c contain a common element (as a main component) in addition to oxygen, a mixed layer with a low density of defect states can be formed. For example, in the case where the metal oxide 230b is an In—Ga—Zn oxide, it is preferable to use an In—Ga—Zn oxide, a Ga—Zn oxide, gallium oxide, or the like as each of the metal oxide 230a and the metal oxide 230c. The metal oxide 230c may have a stacked-layer structure. For example, the metal oxide 230c can have a stacked-layer structure of an In—Ga—Zn oxide and a Ga—Zn oxide over the In—Ga—Zn oxide, or a stacked-layer structure of an In—Ga—Zn oxide and gallium oxide over the In—Ga—Zn oxide. In other words, the metal oxide 230c may have a stacked-layer structure of an In—Ga—Zn oxide and an oxide that does not contain In.

Specifically, as the metal oxide 230a, a metal oxide having an atomic ratio of In:Ga:Zn=1:3:4 [atomic ratio] or In:Ga:Zn=1:1:0.5 [atomic ratio] can be used. As the metal oxide 230b, a metal oxide having an atomic ratio of In:Ga:Zn=4:2:3 [atomic ratio] or In:Ga:Zn=3:1:2 [atomic ratio] can be used. As the metal oxide 230c, a metal oxide having an atomic ratio of In:Ga:Zn=1:3:4 [atomic ratio], In:Ga:Zn=4:2:3 [atomic ratio], Ga:Zn=2:1 [atomic ratio], or Ga:Zn=2:5 [atomic ratio] can be used. Specific examples of a stacked-layer structure of the metal oxide 230c include a stacked-layer structure of a layer having an atomic ratio of In:Ga:Zn=4:2:3 [atomic ratio] and a layer having an atomic ratio of Ga:Zn=2:1 [atomic ratio], a stacked-layer structure of a layer having an atomic ratio of In:Ga:Zn=4:2:3 [atomic ratio] and a layer having an atomic ratio of Ga:Zn=2:5 [atomic ratio], and a stacked-layer structure of a layer having an atomic ratio of In:Ga:Zn=4:2:3 [atomic ratio] and gallium oxide.

At this time, the metal oxide 230b serves as a main carrier path. When the metal oxide 230a and the metal oxide 230c have the above structure, the density of defect states at the interface between the metal oxide 230a and the metal oxide 230b and the interface between the metal oxide 230b and the metal oxide 230c can be made low. This reduces the influence of interface scattering on carrier conduction, and the transistor 200A can have a high on-state current and high frequency characteristics. Note that in the case where the metal oxide 230c has a stacked-layer structure, not only the effect of reducing the density of defect state at the interface between the metal oxide 230b and the metal oxide 230c, but also the effect of inhibiting diffusion of the constituent element of the metal oxide 230c toward the insulator 250 can be expected. Specifically, the metal oxide 230c has a stacked-layer structure in which the upper layer is an oxide that does not contain In, whereby the amount of In that would diffuse toward the insulator 250 can be reduced. Since the insulator 250 functions as a gate insulator, the transistor would show poor characteristics when In diffuses into the insulator 250. Thus, the metal oxide 230c having a stacked-layer structure allows the display device to have high reliability.

The metal oxide 230 is preferably formed using a metal oxide functioning as an oxide semiconductor. For example, the metal oxide to be the channel formation region of the metal oxide 230 has a band gap of preferably 2 eV or higher, further preferably 2.5 eV or higher. The use of a metal oxide having a wide band gap can reduce the off-state current of the transistor. The use of such a transistor can provide a display device with low power consumption.

The conductor 242 (the conductor 242a and the conductor 242b) functioning as the source electrode and the drain electrode is provided over the metal oxide 230b. For the conductor 242, it is preferable to use a metal element selected from aluminum, chromium, copper, silver, gold, platinum, tantalum, nickel, titanium, molybdenum, tungsten, hafnium, vanadium, niobium, manganese, magnesium, zirconium, beryllium, indium, ruthenium, iridium, strontium, and lanthanum; an alloy containing any of the above metal elements; an alloy containing a combination of the above metal elements; or the like. For example, it is preferable to use tantalum nitride, titanium nitride, tungsten, a nitride containing titanium and aluminum, a nitride containing tantalum and aluminum, ruthenium oxide, ruthenium nitride, an oxide containing strontium and ruthenium, an oxide containing lanthanum and nickel, or the like. Tantalum nitride, titanium nitride, a nitride containing titanium and aluminum, a nitride containing tantalum and aluminum, ruthenium oxide, ruthenium nitride, an oxide containing strontium and ruthenium, and an oxide containing lanthanum and nickel are preferable because they are oxidation-resistant conductive materials or materials that retain their conductivity even after absorbing oxygen.

When the conductor 242 is provided in contact with the metal oxide 230, the oxygen concentration of the metal oxide 230 in the vicinity of the conductor 242 sometimes decreases. In addition, a metal compound layer that contains the metal contained in the conductor 242 and the component of the metal oxide 230 is sometimes formed in the metal oxide 230 in the vicinity of the conductor 242. In such cases, the carrier density of the region in the metal oxide 230 in the vicinity of the conductor 242 increases, and the region becomes a low-resistance region.

Here, the region between the conductor 242a and the conductor 242b is formed to overlap with the opening of the insulator 280. In this manner, the conductor 260 can be formed in a self-aligned manner between the conductor 242a and the conductor 242b.

The insulator 250 functions as a gate insulator. The insulator 250 is preferably in contact with a top surface of the metal oxide 230c. For the insulator 250, any of silicon oxide, silicon oxynitride, silicon nitride oxide, silicon nitride, silicon oxide to which fluorine is added, silicon oxide to which carbon is added, silicon oxide to which carbon and nitrogen are added, and porous silicon oxide can be used. In particular, silicon oxide and silicon oxynitride, which have thermal stability, are preferable.

As in the insulator 224, the concentration of impurities such as water or hydrogen in the insulator 250 is preferably reduced. The thickness of the insulator 250 is preferably greater than or equal to 1 nm and less than or equal to 20 nm.

A metal oxide may be provided between the insulator 250 and the conductor 260. The metal oxide preferably has a function of inhibiting oxygen diffusion from the insulator 250 into the conductor 260. Thus, oxidation of the conductor 260 due to oxygen in the insulator 250 can be inhibited.

Note that the metal oxide has a function of part of the gate insulator in some cases. For that reason, when silicon oxide, silicon oxynitride, or the like is used for the insulator 250, the metal oxide is preferably a high-k material with a high dielectric constant. The gate insulator having a stacked-layer structure of the insulator 250 and the metal oxide enables the transistor 200A to be thermally stable and have a high dielectric constant. Accordingly, a gate potential applied during operation of the transistor can be lowered while the physical thickness of the gate insulator is maintained. In addition, the equivalent oxide thickness (EOT) of the insulator functioning as the gate insulator can be reduced.

Specifically, a metal oxide containing one or more of hafnium, aluminum, gallium, yttrium, zirconium, tungsten, titanium, tantalum, nickel, germanium, magnesium, and the like can be used. It is particularly preferable to use an insulator containing an oxide of one or both of aluminum and hafnium, such as aluminum oxide, hafnium oxide, or an oxide containing aluminum and hafnium (hafnium aluminate).

Although the conductor 260 has a two-layer structure in FIG. 39, the conductor 260 may have a single-layer structure or a stacked-layer structure of three or more layers.

The conductor 260a is preferably formed using the aforementioned conductive material having a function of inhibiting diffusion of impurities such as hydrogen atoms, hydrogen molecules, water molecules, nitrogen atoms, nitrogen molecules, nitrogen oxide molecules (e.g., N₂O, NO, and NO₂), and copper atoms. Alternatively, the conductor 260a is preferably formed using a conductive material having a function of inhibiting diffusion of oxygen (e.g., at least one of oxygen atoms and oxygen molecules).

When the conductor 260a has a function of inhibiting diffusion of oxygen, the conductivity of the conductor 260b can be prevented from being lowered because of oxidization of the conductor 260b due to oxygen in the insulator 250. As a conductive material having a function of inhibiting oxygen diffusion, tantalum, tantalum nitride, ruthenium, or ruthenium oxide is preferably used, for example.

The conductor 260b is preferably formed using a conductive material containing tungsten, copper, or aluminum as its main component. The conductor 260 also functions as a wiring and thus is preferably a conductor having high conductivity. For example, a conductive material containing tungsten, copper, or aluminum as its main component can be used. The conductor 260b may have a stacked-layer structure, for example, a stacked-layer structure of titanium or titanium nitride and the above conductive material.

As illustrated in FIG. 39A and FIG. 39C, the side surface of the metal oxide 230 is covered with the conductor 260 in a region where the metal oxide 230b is not overlapped with the conductor 242, that is, the channel formation region of the metal oxide 230. Accordingly, electric fields of the conductor 260 functioning as the first gate electrode are likely to act on the side surface of the metal oxide 230. Hence, the transistor 200A can have a higher on-state current and improved frequency characteristics.

The insulator 254 as well as the insulator 214 and the like preferably functions as a barrier insulating film that inhibits impurities such as water or hydrogen from entering the transistor 200A from the insulator 280 side. For example, it is preferable that the insulator 254 preferably have a lower hydrogen permeability than the insulator 224. Furthermore, as illustrated in FIG. 39B and FIG. 39C, the insulator 254 preferably includes a region in contact with the side surface of the metal oxide 230c, the top surface and side surface of the conductor 242a, the top surface and side surface of the conductor 242b, the side surface of the metal oxide 230a, the side surface of the metal oxide 230b, and the top surface of the insulator 224. Such a structure can inhibit entry of hydrogen of the insulator 280 into the metal oxide 230 through top surfaces or side surfaces of the conductor 242a, the conductor 242b, the metal oxide 230a, the metal oxide 230b, and the insulator 224.

Furthermore, the insulator 254 preferably has a function of inhibiting diffusion of oxygen (e.g., at least one of oxygen atoms and oxygen molecules) (it is preferable that oxygen is less likely to pass through the insulator 254). For example, the insulator 254 preferably has a lower oxygen permeability than the insulator 280 or the insulator 224.

The insulator 254 is preferably formed by a sputtering method. When the insulator 254 is formed by a sputtering method in an oxygen-containing atmosphere, oxygen can be added to a region of the insulator 224 in contact with the insulator 254 and its vicinity. Thus, oxygen can be supplied from the region to the metal oxide 230 through the insulator 224. Here, with the insulator 254 having a function of inhibiting upward oxygen diffusion, diffusion of oxygen from the metal oxide 230 into the insulator 280 can be inhibited. Moreover, with the insulator 222 having a function of inhibiting downward oxygen diffusion, diffusion of oxygen from the metal oxide 230 toward the substrate can be inhibited. In the above manner, oxygen is supplied to the channel formation region of the metal oxide 230. Accordingly, oxygen vacancies in the metal oxide 230 can be reduced, so that the transistor can be prevented from having normally-on characteristics.

As the insulator 254, an insulator containing an oxide of aluminum and/or hafnium is formed, for example. Note that as the insulator containing an oxide of aluminum and/or hafnium, aluminum oxide, hafnium oxide, an oxide containing aluminum and hafnium (hafnium aluminate), or the like is preferably used.

The insulator 224, the insulator 250, and the metal oxide 230 are covered with the insulator 254 having a barrier property against hydrogen, whereby the insulator 280 is isolated from the insulator 224, the metal oxide 230, and the insulator 250 by the insulator 254. This inhibits entry of impurities such as hydrogen from the outside of the transistor 200A, resulting in favorable electrical characteristics and reliability of the transistor 200A.

The insulator 280 is provided over the insulator 224, the metal oxide 230, and the conductor 242 with the insulator 254 placed therebetween. The insulator 280 preferably includes, for example, silicon oxide, silicon oxynitride, silicon nitride oxide, silicon oxide to which fluorine is added, silicon oxide to which carbon is added, silicon oxide to which carbon and nitrogen are added, or porous silicon oxide. Silicon oxide and silicon oxynitride are particularly preferable in terms of high thermal stability. A material such as silicon oxide, silicon oxynitride, or porous silicon oxide is preferably used, in which case a region including oxygen that is released by heating can be easily formed.

The concentration of impurities such as water or hydrogen in the insulator 280 is preferably lowered. The top surface of the insulator 280 may be planarized.

The insulator 274, like the insulator 214 or the like, preferably functions as a barrier insulating film that inhibits entry of impurities such as water and hydrogen into the insulator 280. The insulator 274 can be formed using an insulator that can be used as the insulator 214 or the insulator 254, for example.

The insulator 281 functioning as an interlayer film is preferably provided over the insulator 274. As in the insulator 224 or the like, the concentration of impurities such as water and hydrogen in the insulator 281 is preferably reduced.

The conductor 240a and the conductor 240b are provided in openings formed in the insulator 281, the insulator 274, the insulator 280, and the insulator 254. The conductor 240a and the conductor 240b are positioned to face each other with the conductor 260 therebetween. Note that the top surfaces of the conductor 240a and the conductor 240b may be level with the top surface of the insulator 281.

The insulator 241a is provided in contact with the inner wall of the opening in the insulator 281, the insulator 274, the insulator 280, and the insulator 254, and the first conductor of the conductor 240a is formed in contact with the side surface of the insulator 241a. The conductor 242a is positioned on at least part of the bottom of the opening, and thus the conductor 240a is in contact with the conductor 242a. Similarly, the insulator 241b is provided in contact with the inner wall of another opening in the insulator 281, the insulator 274, the insulator 280, and the insulator 254, and the first conductor of the conductor 240b is formed in contact with the side surface of the insulator 241b. The conductor 242b is positioned on at least part of the bottom of the opening, and thus the conductor 240b is in contact with the conductor 242b.

The conductor 240a and the conductor 240b are preferably formed using a conductive material containing tungsten, copper, or aluminum as its main component. The conductor 240a and the conductor 240b may have a stacked-layer structure.

When the conductor 240 has a stacked-layer structure, the aforementioned conductor having a function of inhibiting diffusion of impurities such as water or hydrogen is preferably used for the conductor in contact with the metal oxide 230a, the metal oxide 230b, the conductor 242, the insulator 254, the insulator 280, the insulator 274, and the insulator 281. For example, tantalum, tantalum nitride, titanium, titanium nitride, ruthenium, or ruthenium oxide is preferably used. The conductive material having a function of inhibiting diffusion of impurities such as water or hydrogen can be used as a single layer or stacked layers. The use of the conductive material can prevent oxygen added to the insulator 280 from being absorbed by the conductor 240a and the conductor 240b, and prevent impurities such as water or hydrogen from entering the metal oxide 230 through the conductor 240a and the conductor 240b from the components above the insulator 281.

The insulator 241a and the insulator 241b are formed using any of the insulators that can be used for the insulator 254, for example. Since the insulator 241a and the insulator 241b are provided in contact with the insulator 254, impurities such as water and hydrogen in the insulator 280 or the like can be prevented from entering the metal oxide 230 through the conductor 240a and the conductor 240b. Furthermore, oxygen contained in the insulator 280 can be prevented from being absorbed by the conductor 240a and the conductor 240b.

Although not illustrated, a conductor functioning as a wiring may be provided in contact with the top surface of the conductor 240a and the top surface of the conductor 240b. The conductor functioning as a wiring is preferably formed using a conductive material containing tungsten, copper, or aluminum as its main component. The conductor may have a stacked-layer structure, for example, a stack of titanium or titanium nitride and the above conductive material. Note that the conductor may be formed to be embedded in an opening provided in an insulator.

### Structure Example 2 of Transistor

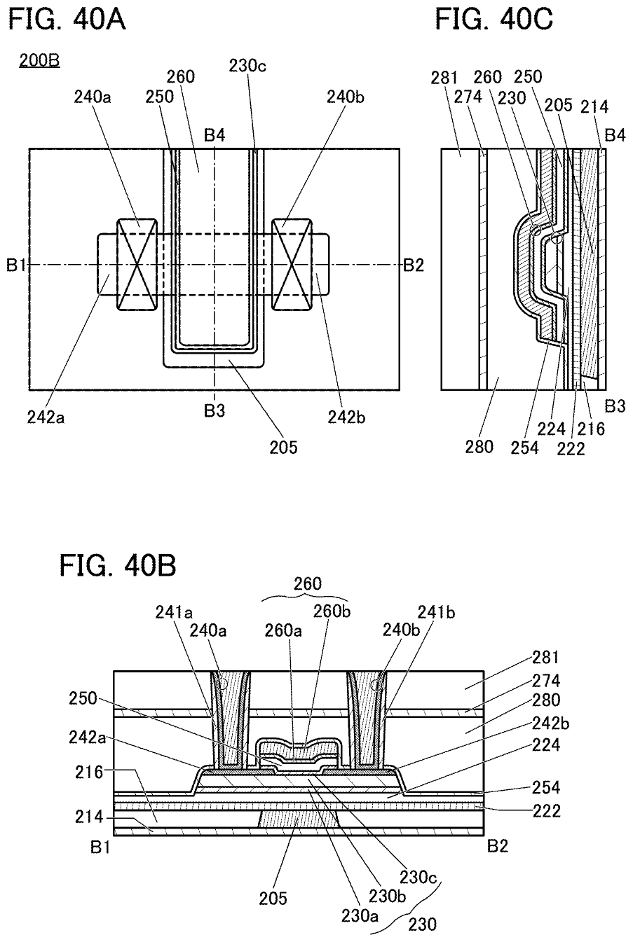

FIG. 40A, FIG. 40B, and FIG. 40C are a top view and cross-sectional views of a transistor 200B that can be used in the display device of one embodiment of the present invention, and the periphery of the transistor 200B. The transistor 200B is a variation example of the transistor 200A.

FIG. 40A is a top view of the transistor 200B. FIG. 40B and FIG. 40C are cross-sectional views of the transistor 200B. FIG. 40B is a cross-sectional view taken along the dashed-dotted line B1-B2 in FIG. 40A and shows a cross section of the transistor 200B in the channel length direction. FIG. 40C is a cross-sectional view taken along the dashed-dotted line B3-B4 in FIG. 40A and shows a cross section of the transistor 200B in the channel width direction. Note that for simplification of the drawing, some components are not illustrated in the top view in FIG. 40A.

In the transistor 200B, the conductor 242a and the conductor 242b each have a region overlapping with the metal oxide 230c, the insulator 250, and the conductor 260. Thus, the transistor 200B can have a high on-state current. In addition, the transistor 200B can be a transistor that is easy to control.

The conductor 260 functioning as a gate electrode includes the conductor 260a and the conductor 260b over the conductor 260a. The conductor 260a is preferably formed using a conductive material having a function of inhibiting diffusion of impurities such as a hydrogen atom, a hydrogen molecule, a water molecule, and a copper atom. Alternatively, the conductor 260a is preferably formed using a conductive material having a function of inhibiting diffusion of oxygen (e.g., at least one of oxygen atoms and oxygen molecules).

When the conductor 260a has a function of inhibiting oxygen diffusion, the range of choices for the material of the conductor 260b can be expanded. That is, the conductor 260a inhibits oxidation of the conductor 260b, thereby inhibiting the decrease in conductivity.

The insulator 254 is preferably provided to cover the top surface and the side surface of the conductor 260, the side surface of the insulator 250, and the side surface of the metal oxide 230c. Note that the insulator 254 is preferably formed using an insulating material having a function of inhibiting diffusion of oxygen and impurities such as water or hydrogen.

The insulator 254 can inhibit oxidation of the conductor 260. Moreover, the insulator 254 can inhibit diffusion of impurities such as water and hydrogen contained in the insulator 280 into the transistor 200B.

### Structure Example 3 of Transistor

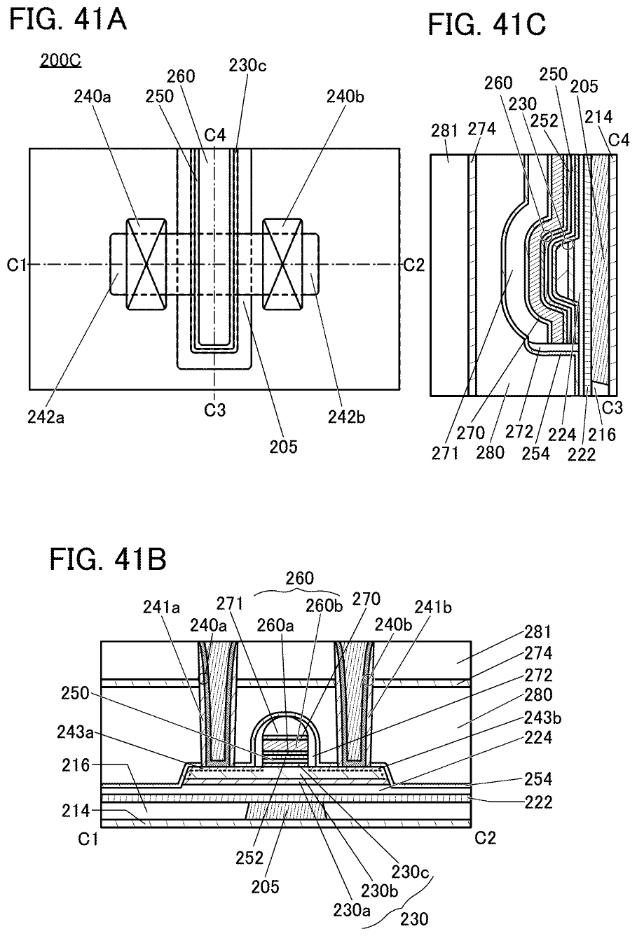

FIG. 41A, FIG. 41B, and FIG. 41C are a top view and cross-sectional views of a transistor 200C that can be used in the display device of one embodiment of the present invention, and the periphery of the transistor 200C. The transistor 200C is a variation example of the transistor 200A.

FIG. 41A is a top view of the transistor 200C. FIG. 41B and FIG. 41C are cross-sectional views of the transistor 200C. FIG. 41B is a cross-sectional view taken along the dashed-dotted line C1-C2 in FIG. 41A and shows a cross section of the transistor 200C in the channel length direction. FIG. 41C is a cross-sectional view taken along the dashed-dotted line C3-C4 in FIG. 41A and shows a cross section of the transistor 200C in the channel width direction. Note that for simplification of the drawing, some components are not illustrated in the top view in FIG. 41A.

The transistor 200C includes the insulator 250 over the metal oxide 230c, a metal oxide 252 over the insulator 250, the conductor 260 over the metal oxide 252, an insulator 270 over the conductor 260, and an insulator 271 over the insulator 270.

The metal oxide 252 preferably has a function of inhibiting diffusion of oxygen. When the metal oxide 252 that inhibits oxygen diffusion is provided between the insulator 250 and the conductor 260, diffusion of oxygen into the conductor 260 is inhibited. That is, the reduction in the amount of oxygen supplied to the metal oxide 230 can be inhibited. Furthermore, oxidation of the conductor 260 can be inhibited.

Note that the metal oxide 252 may function as part of a gate electrode. For example, an oxide semiconductor that can be used for the metal oxide 230 can be used for the metal oxide 252. In this case, when the conductor 260 is formed by a sputtering method, the metal oxide 252 can have a reduced electric resistance and become a conductor. Such a conductor can be referred to as an oxide conductor (OC) electrode.

Note that the metal oxide 252 may function as part of a gate insulator. Therefore, when silicon oxide, silicon oxynitride, or the like, which has high thermal stability, is used for the insulator 250, a metal oxide that is a high-k material with a high dielectric constant is preferably used as the metal oxide 252. This stacked-layer structure enables the transistor 200C to be thermally stable and have a high dielectric constant. Accordingly, a gate potential that is applied during operation of the transistor can be lowered while the physical thickness is maintained. In addition, the equivalent oxide thickness (EOT) of the insulator functioning as the gate insulator can be reduced.

Although the metal oxide 252 in the transistor 200C is shown as a single layer, the metal oxide 252 may have a stacked-layer structure of two or more layers. For example, a metal oxide functioning as part of a gate electrode and a metal oxide functioning as part of a gate insulator may be stacked.

When the metal oxide 252 included in the transistor 200C functions as a gate electrode, the on-state current of the transistor 200C can be increased without weakening the influence of electric fields from the conductor 260. When the metal oxide 252 functions as a gate insulator, the distance between the conductor 260 and the metal oxide 230 can be maintained owing to the physical thickness of the insulator 250 and the metal oxide 252. Thus, leakage current between the conductor 260 and the metal oxide 230 can be reduced. Consequently, in the transistor 200C having the stacked-layer structure of the insulator 250 and the metal oxide 252, it is easy to adjust the physical distance between the conductor 260 and the metal oxide 230 and the intensity of electric fields applied from the conductor 260 to the metal oxide 230.

Specifically, for the metal oxide 252, a material obtained by lowering the resistance of an oxide semiconductor that can be used for the metal oxide 230 can be used. Alternatively, a metal oxide containing one or more of hafnium, aluminum, gallium, yttrium, zirconium, tungsten, titanium, tantalum, nickel, germanium, magnesium, and the like can be used.

It is particularly preferable to use an insulator containing an oxide of one or both of aluminum and hafnium, such as aluminum oxide, hafnium oxide, or an oxide containing aluminum and hafnium (hafnium aluminate). In particular, hafnium aluminate is preferable because it has higher heat resistance than hafnium oxide and thus is less likely to be crystallized by heat treatment in a later step. Note that the metal oxide 252 is not necessarily provided. Design is appropriately determined in consideration of required transistor characteristics.

The insulator 270 is preferably formed using an insulating material having a function of inhibiting the passage of oxygen and impurities such as water or hydrogen. For example, aluminum oxide or hafnium oxide is preferably used. In that case, oxidization of the conductor 260 due to oxygen from above the insulator 270 can be inhibited. Moreover, entry of impurities such as water or hydrogen from above the insulator 270 into the metal oxide 230 through the conductor 260 and the insulator 250 can be inhibited.

The insulator 271 functions as a hard mask. By provision of the insulator 271, the conductor 260 can be processed to have a side surface that is substantially perpendicular. Specifically, the angle formed by the side surface of the conductor 260 and the surface of the substrate can be greater than or equal to 75° and less than or equal to 100°, preferably greater than or equal to 80° and less than or equal to 95°.

The insulator 271 may be formed using an insulating material having a function of inhibiting the passage of oxygen and impurities such as water or hydrogen so that the insulator 271 also functions as a barrier layer. In this case, the insulator 270 is not necessarily provided.

The insulator 270, the conductor 260, the metal oxide 252, the insulator 250, and the metal oxide 230c are selectively removed using the insulator 271 as a hard mask, whereby their side surfaces can be substantially aligned with each other and the surface of the metal oxide 230b can be partly exposed.

The transistor 200C includes a region 243a and a region 243b on part of the exposed surface of the metal oxide 230b. One of the region 243a and the region 243b functions as a source region, and the other of the region 243a and the region 243b functions as a drain region.

The region 243a and the region 243b can be formed by addition of an impurity element such as phosphorus or boron to the exposed surface of the metal oxide 230b by an ion implantation method, an ion doping method, a plasma immersion ion implantation method, or plasma treatment, for example. In this embodiment and the like, an impurity element refers to an element other than main constituent elements.

Alternatively, the region 243a and the region 243b can be formed in such manner that, after part of the surface of the metal oxide 230b is exposed, a metal film is formed and then heat treatment is performed so that the element contained in the metal film is diffused into the metal oxide 230b.

The electrical resistivity of the regions of the metal oxide 230b to which the impurity element is added decreases. For that reason, the region 243a and the region 243b are sometimes referred to as “impurity regions” or “low-resistance regions”.

The region 243a and the region 243b can be formed in a self-aligned manner by using the insulator 271 and/or the conductor 260 as a mask. Accordingly, the conductor 260 does not overlap the region 243a and/or the region 243b, so that the parasitic capacitance can be reduced. Moreover, an offset region is not formed between the channel formation region and the source-drain region (the region 243a or the region 243b). The formation of the region 243a and the region 243b in a self-aligned manner achieves a higher on-state current, a lower threshold voltage, and a higher operating frequency, for example.

The transistor 200C includes an insulator 272 on the side surfaces of the insulator 271, the insulator 270, the conductor 260, the metal oxide 252, the insulator 250, and the metal oxide 230c. The insulator 272 is preferably an insulator having a low dielectric constant. The insulator 272 is preferably silicon oxide, silicon oxynitride, silicon nitride oxide, silicon nitride, silicon oxide to which fluorine is added, silicon oxide to which carbon is added, silicon oxide to which carbon and nitrogen are added, porous silicon oxide, or a resin, for example. In particular, silicon oxide, silicon oxynitride, silicon nitride oxide, or porous silicon oxide is preferably used for the insulator 272 because an excess oxygen region can be easily formed in the insulator 272 in a later step. Silicon oxide and silicon oxynitride are preferable because of their thermal stability. The insulator 272 preferably has a function of diffusing oxygen.

Note that an offset region may be provided between the channel formation region and the source-drain region in order to further reduce the off-state current. The offset region is a region where the electrical resistivity is high and the impurity element is not added. The offset region can be formed by addition of the impurity element after the formation of the insulator 272. In this case, the insulator 272 serves as a mask like the insulator 271 or the like. Thus, the impurity element is not added to the region of the metal oxide 230b overlapped by the insulator 272, so that the electrical resistivity of the region can be kept high.

The transistor 200C also includes the insulator 254 over the insulator 272 and the metal oxide 230. The insulator 254 is preferably formed by a sputtering method. The insulator formed by a sputtering method can be an insulator containing few impurities such as water or hydrogen.

Note that an oxide film formed by a sputtering method may extract hydrogen from the component over which the oxide film is formed. For that reason, the insulator 254 formed by a sputtering method absorbs hydrogen and water from the metal oxide 230 and the insulator 272. This reduces the hydrogen concentration in the metal oxide 230 and the insulator 272.

<Materials for Transistor>

Materials that can be used for the transistor will be described.

<<Substrate>>

As a substrate where the transistor 200A, the transistor 200B, or the transistor 200C is formed, an insulator substrate, a semiconductor substrate, or a conductor substrate can be used, for example. Examples of the insulator substrate include a glass substrate, a quartz substrate, a sapphire substrate, a stabilized zirconia substrate (e.g., an yttria-stabilized zirconia substrate), and a resin substrate. Examples of the semiconductor substrate include a semiconductor substrate of silicon or germanium and a compound semiconductor substrate of silicon carbide, silicon germanium, gallium arsenide, indium phosphide, zinc oxide, or gallium oxide. Another example includes a semiconductor substrate in which an insulator region is provided in the above semiconductor substrate, e.g., a silicon on insulator (SOI) substrate. Examples of the conductor substrate include a graphite substrate, a metal substrate, an alloy substrate, and a conductive resin substrate. Other examples include a substrate containing a nitride of a metal, a substrate including an oxide of a metal, an insulator substrate provided with a conductor or a semiconductor, a semiconductor substrate provided with a conductor or an insulator, and a conductor substrate provided with a semiconductor or an insulator. Alternatively, any of these substrates provided with an element may be used. Examples of the element provided over the substrate include a capacitor, a resistor, a switching element, and a memory element.

A flexible substrate may be used as the substrate, and the transistor 200A, the transistor 200B, or the transistor 200C may be provided directly on the flexible substrate. Alternatively, a separation layer may be provided between the substrate and the transistor. The separation layer can be used when part or the whole of the transistor formed over the separation layer is separated from the substrate and transferred to another substrate. Thus, the transistor can be transferred to a substrate having low heat resistance or a flexible substrate.

<<Insulator>>

Examples of an insulator include an insulating oxide, an insulating nitride, an insulating oxynitride, an insulating nitride oxide, an insulating metal oxide, an insulating metal oxynitride, and an insulating metal nitride oxide.

With miniaturization and high integration of transistors, for example, a problem such as generation of leakage current may arise because of a thin gate insulator. When a high-k material is used for an insulator functioning as a gate insulator, the driving voltage of the transistor can be lowered while the physical thickness of the gate insulator is kept. On the other hand, when a material having a low dielectric constant is used for an insulator functioning as an interlayer film, the parasitic capacitance between wirings can be reduced. Accordingly, a material is preferably selected depending on the function of an insulator.

Examples of the insulator having a high dielectric constant include gallium oxide, hafnium oxide, zirconium oxide, an oxide containing aluminum and hafnium, an oxynitride containing aluminum and hafnium, an oxide containing silicon and hafnium, an oxynitride containing silicon and hafnium, and a nitride containing silicon and hafnium.

Examples of the insulator having a low dielectric constant include silicon oxide, silicon oxynitride, silicon nitride oxide, silicon nitride, silicon oxide to which fluorine is added, silicon oxide to which carbon is added, silicon oxide to which carbon and nitrogen are added, porous silicon oxide, and a resin.

When a transistor including an oxide semiconductor is surrounded by insulators having a function of inhibiting transmission of oxygen and impurities such as hydrogen (e.g., the insulator 214, the insulator 222, the insulator 254, and the insulator 274), the electrical characteristics of the transistor can be stable. An insulator with a function of inhibiting transmission of oxygen and impurities such as hydrogen can be formed to have a single-layer structure or a stacked-layer structure including an insulator containing, for example, boron, carbon, nitrogen, oxygen, fluorine, magnesium, aluminum, silicon, phosphorus, chlorine, argon, gallium, germanium, yttrium, zirconium, lanthanum, neodymium, hafnium, or tantalum. Specifically, as the insulator with a function of inhibiting transmission of oxygen and impurities such as hydrogen, a metal oxide such as aluminum oxide, magnesium oxide, gallium oxide, germanium oxide, yttrium oxide, zirconium oxide, lanthanum oxide, neodymium oxide, hafnium oxide, or tantalum oxide or a metal nitride such as aluminum nitride, aluminum titanium nitride, titanium nitride, silicon nitride oxide, or silicon nitride can be used.

An insulator functioning as a gate insulator preferably includes a region containing oxygen that is released by heating. For example, when silicon oxide or silicon oxynitride that includes a region containing oxygen released by heating is provided in contact with the metal oxide 230, oxygen vacancies in the metal oxide 230 can be compensated.

<<Conductor>>

For the conductor, it is preferable to use a metal element selected from aluminum, chromium, copper, silver, gold, platinum, tantalum, nickel, titanium, molybdenum, tungsten, hafnium, vanadium, niobium, manganese, magnesium, zirconium, beryllium, indium, ruthenium, iridium, strontium, lanthanum, and the like; an alloy containing any of the above metal elements; an alloy containing a combination of the above metal elements; or the like. For example, it is preferable to use tantalum nitride, titanium nitride, tungsten, a nitride containing titanium and aluminum, a nitride containing tantalum and aluminum, ruthenium oxide, ruthenium nitride, an oxide containing strontium and ruthenium, an oxide containing lanthanum and nickel, or the like. Tantalum nitride, titanium nitride, a nitride containing titanium and aluminum, a nitride containing tantalum and aluminum, ruthenium oxide, ruthenium nitride, an oxide containing strontium and ruthenium, and an oxide containing lanthanum and nickel are preferable because they are oxidation-resistant conductive materials or materials that maintain their conductivity even after absorbing oxygen. In addition, a semiconductor having high electric conductivity, typified by polycrystalline silicon containing an impurity element such as phosphorus, or silicide such as nickel silicide may be used.

Conductors formed using any of the above materials may be stacked. For example, a stacked-layer structure combining a material containing any of the above metal elements and a conductive material containing oxygen may be used. A stacked-layer structure combining a material containing any of the above metal elements and a conductive material containing nitrogen may be used. Further, a stacked-layer structure combining a material containing any of the above metal elements, a conductive material containing oxygen, and a conductive material containing nitrogen may be used.

When a metal oxide is used for the channel formation region of the transistor, the conductor functioning as the gate electrode preferably employs a stacked-layer structure using a material containing any of the above metal elements and a conductive material containing oxygen. In this case, the conductive material containing oxygen is preferably provided on the channel formation region side. When the conductive material containing oxygen is provided on the channel formation region side, oxygen released from the conductive material is easily supplied to the channel formation region.

It is particularly preferable to use, for the conductor functioning as the gate electrode, a conductive material containing oxygen and a metal element contained in the metal oxide in which the channel is formed. A conductive material containing any of the above metal elements and nitrogen may be used. For example, a conductive material containing nitrogen, such as titanium nitride or tantalum nitride, may be used. Indium tin oxide, indium oxide containing tungsten oxide, indium zinc oxide containing tungsten oxide, indium oxide containing titanium oxide, indium tin oxide containing titanium oxide, indium zinc oxide, or indium tin oxide to which silicon is added may be used. Indium gallium zinc oxide containing nitrogen may be used. With the use of such a material, hydrogen contained in the metal oxide in which the channel is formed can be captured in some cases. Alternatively, hydrogen entering from a surrounding insulator or the like can be captured in some cases.

<<Metal Oxide>>

A metal oxide contains preferably at least indium or zinc and particularly preferably indium and zinc. In addition, aluminum, gallium, yttrium, tin, or the like is preferably contained. Furthermore, one or more elements selected from boron, titanium, iron, nickel, germanium, zirconium, molybdenum, lanthanum, cerium, neodymium, hafnium, tantalum, tungsten, magnesium, and the like may be contained.

Here, the case where the metal oxide is an In-M-Zn oxide that contains indium, an element M, and zinc is considered. The element M is aluminum, gallium, yttrium, tin, or the like. Other examples that can be used as the element M include boron, titanium, iron, nickel, germanium, zirconium, molybdenum, lanthanum, cerium, neodymium, hafnium, tantalum, tungsten, and magnesium. Note that two or more of the above elements can be used in combination as the element Min some cases.

Note that in this specification and the like, a metal oxide containing nitrogen is also referred to as a metal oxide in some cases. A metal oxide containing nitrogen may be referred to as a metal oxynitride.

[Structure of Metal Oxide]

An oxide semiconductor (metal oxide) is classified into a single crystal oxide semiconductor and a non-single-crystal oxide semiconductor. Examples of a non-single-crystal oxide semiconductor include a CAAC-OS, a polycrystalline oxide semiconductor, an nc-OS (nanocrystalline oxide semiconductor), an a-like OS (amorphous-like oxide semiconductor), and an amorphous oxide semiconductor.

[Impurities]

Here, the influence of impurities in the metal oxide is described. When the metal oxide contains an alkali metal or an alkaline earth metal, defect states are formed and carriers are generated in some cases. Thus, a transistor using a metal oxide containing an alkali metal or an alkaline earth metal in a channel formation region tends to have normally-on characteristics. Therefore, it is preferable to reduce the concentration of an alkali metal or an alkaline earth metal in the metal oxide. Specifically, the concentration of an alkali metal or an alkaline earth metal in the metal oxide, measured by secondary ion mass spectrometry (SIMS), is lower than or equal to 1×10¹⁸ atoms/cm³, preferably lower than or equal to 2×10¹⁶ atoms/cm³.

Hydrogen contained in a metal oxide reacts with oxygen bonded to a metal atom and forms water. Hence, hydrogen contained in a metal oxide may cause oxygen vacancies in the metal oxide. Entry of hydrogen into the oxygen vacancies generates electrons serving as carriers in some cases. Furthermore, some hydrogen may react with oxygen bonded to a metal atom and generate an electron serving as a carrier. Thus, a transistor including a metal oxide that contains hydrogen tends to have normally-on characteristics.

For this reason, hydrogen in the metal oxide is preferably reduced as much as possible. Specifically, the hydrogen concentration of the metal oxide measured by SIMS is lower than 1×10²⁰ atoms/cm³, preferably lower than 1×10¹⁹ atoms/cm³, further preferably lower than 5×10¹⁸ atoms/cm³, still further preferably lower than 1×10¹⁸ atoms/cm³. When a metal oxide with a sufficiently reduced impurity concentration is used for a channel formation region of a transistor, the transistor can have stable electrical characteristics.

As a metal oxide used for a semiconductor of a transistor, a thin film having high crystallinity is preferably used. With the thin film, the stability or reliability of the transistor can be improved. As the thin film, a thin film of a single crystal metal oxide or a thin film of a polycrystalline metal oxide can be used, for example. However, a high-temperature process or a laser heating process is required to form the thin film of a single crystal metal oxide or the thin film of a polycrystalline metal oxide over a substrate. Thus, the manufacturing cost is increased, and the throughput is decreased.

At least part of any of the structure examples, the drawings corresponding thereto, and the like described in this embodiment can be implemented in combination with any of the other structure examples, the other drawings, and the like as appropriate.

At least part of this embodiment can be implemented in combination with any of the other embodiments described in this specification as appropriate.

### REFERENCE NUMERALS

10: electronic device, 10a: electronic device, 10b: electronic device, 11: housing, 11a: first part, 11b: second part, 11c: third part, 11d: fourth part, 11e: fifth part, 11f: sixth part, 11g: seventh part, 11h: eighth part, 11i: ninth part, 12a: first portion, 12b: second portion, 12c: third portion, 12d: fourth portion, 12e: fifth portion, 13: display device, 13L: display device, 13R: display device, 15L: optical component, 15R: optical component, 17: detection device, 17L: detection device, 17R: detection device, 18: memory device, 19: arithmetic device, 21: input/output device, 23: server, 25: fastening, 27: buckle catch, 29: separator, 31: electronic device, 33: display portion, 41: space, 43: opening portion, 45: adjustment mechanism, 53: feature-extraction unit, 54: estimation unit, 55: information-generation unit, 61: input layer, 62: intermediate layer, 63: output layer, 71: data, 72: data, 73: data, 74: data, 81: information, 82: information, 91: information, 92: information, 93: information, 94: information, 95: information, 200A: transistor, 200B: transistor, 200C: transistor, 205: conductor, 214: insulator, 216: insulator, 222: insulator, 224: insulator, 230: metal oxide, 230a: metal oxide, 230b: metal oxide, 230c: metal oxide, 240: conductor, 240a: conductor, 240b: conductor, 241: insulator, 241a: insulator, 241b: insulator, 242: conductor, 242a: conductor, 242b: conductor, 243a: region, 243b: region, 244: insulator, 250: insulator, 252: metal oxide, 254: insulator, 260: conductor, 260a: conductor, 260b: conductor, 270: insulator, 271: insulator, 272: insulator, 274: insulator, 280: insulator, 281: insulator, 301a: conductor, 301b: conductor, 305: conductor, 311: conductor, 313: conductor, 317: conductor, 321: lower electrode, 323: insulator, 325: upper electrode, 331: conductor, 333: conductor, 335: conductor, 337: conductor, 341: conductor, 343: conductor, 347: conductor, 351: conductor, 353: conductor, 355: conductor, 357: conductor, 361: insulator, 363: insulator, 401: circuit, 403: element isolation layer, 40 5: insulator, 407: insulator, 409: insulator, 411: insulator, 413: insulator, 415: insulator, 417: insulator, 419: insulator, 421: insulator, 441: transistor, 443: conductor, 445: insulator, 447: semiconductor region, 449a: low-resistance region, 449b: low-resistance region, 451: conductor, 453: conductor, 455: conductor, 457: conductor, 459: conductor, 461: conductor, 463: conductor, 465: conductor, 467: conductor, 469: conductor, 471: conductor, 501: insulator, 503: insulator, 505: insulator, 507: insulator, 509: insulator, 511: transistor, 513: transistor, 515: capacitor, 517: capacitor, 520: circuit, 521: transistor, 525: transistor, 527: transistor, 529: transistor, 535: wiring, 537: wiring, 539: wiring, 541: wiring, 543: wiring, 545: wiring, 552: transistor, 554: transistor, 562: capacitor, 572: light-emitting device, 572_1: light-emitting device, 572_2: light-emitting device, 601: transistor, 602: transistor, 603: transistor, 613: insulator, 614: insulator, 616: insulator, 622: insulator, 624: insulator, 644: insulator, 654: insulator, 674: insulator, 680: insulator, 681: insulator, 701: substrate, 705: substrate, 712: sealant, 716: FPC, 721: hole-injection layer, 722: hole-transport layer, 723: light-emitting layer, 724: electron-transport layer, 725: electron-injection layer, 730: insulator, 732: sealing layer, 734: insulator, 736: coloring layer, 738: light-blocking layer, 750: transistor, 760: connection electrode, 772: conductor, 778: component, 780: anisotropic conductor, 786: EL layer, 786a: EL layer, 786b: EL layer, 786c: EL layer, 788: conductor, 790: capacitor, 792: charge generation layer, 810: display device, 820: layer, 821: gate driver circuit, 822: source driver circuit, 823: region, 824: demultiplexer circuit, 830: layer, 831: wiring, 831-1: wiring, 831-2: wiring, 831_1: wiring, 831_2: wiring, 832: wiring, 832-1: wiring, 832-2: wiring, 832_1: wiring, 832_2: wiring, 833: pixel array, 834: pixel, 835a: wiring, 835b: wiring, 840: circuit, 993IR: coloring layer, 993R: coloring layer, 995: substrate, 1001: substrate, 1002: insulator, 1003: transistor, 1004: insulator, 1005: insulator, 1010: photoelectric conversion device, 1010_1: photoelectric conversion device, 1010_2: photoelectric conversion device, 1011: active layer

1. An electronic device comprising a detection device, an arithmetic device, and a housing,
wherein the housing comprises a space at a position overlapping with a user's nose when the user wears the electronic device,
wherein the detection device is located between the housing and the user's nose,
wherein the detection device is configured to obtain user's data on an emotion of the user and output the user's data to the arithmetic device, and
wherein the arithmetic device is configured to generate display data based on the user's data and outputting the display data.

2. An electronic device comprising a detection device, an arithmetic device, and a housing,
wherein the housing comprises a space at a position overlapping with a user's nose when the user wears the electronic device,
wherein the detection device is located in the inside of the housing to overlap with the user's nose,
wherein the detection device is configured to obtain user's data on an emotion of the user and output the user's data to the arithmetic device, and
wherein the arithmetic device is configured to generate display data based on the user's data and outputting the display data.

3. (canceled)

4. (canceled)

5. The electronic device according to claim 1, wherein the detection device comprises at least one of a temperature sensor, a humidity sensor, a microphone, and an imaging device.

6. The electronic device according to claim 1, wherein the user's data is at least one of a temperature, a humidity, a sound, and an image.

7. The electronic device according to claim 1, further comprising an adjustment mechanism,
wherein the adjustment mechanism is configured to adjust an angle of the detection device with respect to the housing.

8. The electronic device according to claim 1,
wherein the detection device comprises an imaging device,
wherein the detection device is configured to output, to the arithmetic device, a captured image of the user as the user's data, and
wherein the arithmetic device is configured to estimate an emotion of the user from the user's data and generate the display data based on the estimated emotion.

9. The electronic device according to claim 8, wherein the user's data is an image of a portion including the user's nose.

10. The electronic device according to claim 8, wherein the user's data is an image of a portion including the user's mouth.

11. The electronic device according to claim 8, wherein a neural network is used for the estimation.

12. The electronic device according to claim 1, further comprising a display device,
wherein the arithmetic device is configured to output the display data to the display device.

13. The electronic device according to claim 2, wherein the detection device comprises at least one of a temperature sensor, a humidity sensor, a microphone, and an imaging device.

14. The electronic device according to claim 2, wherein the user's data is at least one of a temperature, a humidity, a sound, and an image.

15. The electronic device according to claim 2, further comprising an adjustment mechanism,
wherein the adjustment mechanism is configured to adjust an angle of the detection device with respect to the housing.

16. The electronic device according to claim 2,
wherein the detection device comprises an imaging device,
wherein the detection device is configured to output, to the arithmetic device, a captured image of the user as the user's data, and
wherein the arithmetic device is configured to estimate an emotion of the user from the user's data and generate the display data based on the estimated emotion.

17. The electronic device according to claim 16, wherein the user's data is an image of a portion including the user's nose.

18. The electronic device according to claim 16, wherein the user's data is an image of a portion including the user's mouth.

19. The electronic device according to claim 16, wherein a neural network is used for the estimation.

20. The electronic device according to claim 2, further comprising a display device,
wherein the arithmetic device is configured to output the display data to the display device.
